(19) United States
(12) Patent Application Publication
(10) Pub. No.: US 2005/0118570 A1
(43) Pub. Date: Jun. 2, 2005

(54) Sample preparation methods and devices

(75) Inventors: Mark A. Hollis, Concord, MA (US); Nicholas Matthew Freeland Judson, Somerville, MA (US); Christina Marie Rudzinski, Belmont, MA (US); Lalitha Parameswaran, Billerica, MA (US); Theodore H. Fedynyshyn, Sudbury, MA (US); Catherine Regina Cabrera, Cambridge, MA (US); Laura T. Bortolin, Devens, MA (US)

Correspondence Address: FISH & NEAVE IP GROUP;ROPES & GRAY LLP, ONE INTERNATIONAL PLACE, BOSTON, MA, 02110-2624, US

(73) Assignee: Massachusetts Institute of Technology, Cambridge, MA (US)

(21) Appl. No.: 10/916,784

(22) Filed: Aug. 12, 2004

Related U.S. Application Data
(60) Provisional application No. 60/494,702, filed on Aug. 12, 2003.

(51) Int. Cl. C12Q 1/70; C12Q 1/68; G01N 33/53; C12M 1/34

(52) U.S. Cl. 435/5; 435/6; 435/7.1; 435/287.2

(57) ABSTRACT

The present invention provides improved methods, compositions, and devices for separating and/or detecting targets from biological, environmental, or chemical samples.

### RELATED APPLICATION

This application claims priority to U.S. Application No. 60/494,702, filed Aug. 12, 2003, the disclosure of which is hereby incorporated by reference in its entirety.

### GOVERNMENT SUPPORT

This invention was supported, in whole or in part, by Lincoln Contract Number F19628-95-C-0002 from Defense Directorate of Research and Engineering. The Government has certain rights in the invention.

### BACKGROUND

Biological, chemical, and environmental studies often require the separation of particular targets from amongst a heterogeneous population of materials. Often, the separation of a particular target, as well as its further analysis, are hindered by factors including (a) a very low concentration of the target within the heterogeneous starting mixture of materials, (b) the presence of agents which degrade the target, (c) the presence of agents which interfere with the isolation of the target, and (d) the presence of agents which interfere with the analysis of target following its isolation. The most advantageous methods and compositions facilitate the separation of low concentrations of target from a wide range of either liquid or solid samples containing a heterogeneous mixtures of non-target materials. Such methods and compositions may be further modified or combined with existing methodologies to help maintain the integrity of the target (e.g., prevent its degradation or contamination) and/or to inhibit the activity of agents which interfere with the further analysis of the target (e.g., agents which interfere with PCR analysis of DNA samples, agents which interfere with mass spectroscopic analysis of protein samples, or agents which interfere with cytological analysis of bacteria or viruses).

Advances in fields including cell biology, molecular biology, chemistry, toxicology, and pharmacology have spawned a variety of techniques for analyzing biological materials, chemical materials, and environmental materials including, but not limited to, DNA, RNA, protein, bacterial cells and spores (including gram+ and gram−), viruses (including DNA based and RNA based), small organic molecules, and large chemical compounds. However, the efficient application of many powerful analytical tools is often hindered by an inability to separate a target material of interest away from a heterogeneous population of materials contained in a sample. The present invention provides methods, compositions, and apparatuses to facilitate the separation and/or identification of targets from environmental, biological, and chemical samples.

### SUMMARY

The present invention provides methods, compositions, and apparatuses which can be used to separate and/or identify a target from a heterogeneous mixture of agents. Separation of a target, which may be DNA, RNA, protein, bacterial cells or spores, viruses, small organic molecules, or chemical compounds, facilitates further analysis and identification of the target. The present invention has a wide range of forensic, medical, environmental, industrial, public health, and anti-bioterrorism applications, and is suitable for use in separating targets from a wide range of gaseous, liquid, and solid samples.

In a first aspect, the present invention provides an improved method for separating a target from a heterogeneous sample. In one embodiment, the method comprises contacting the sample containing a target of interest with a substrate capable of binding the target with a higher affinity than the affinity of the substrate for non-target materials. In another embodiment, the surface of the substrate is coated with a modifying agent that further increases the affinity of the substrate for one or more particular targets. In another embodiment, the substrate is coated with one or more of the amine containing modifying agents disclosed herein. The use of either magnetic or non-magnetic substrates coated with one or more simple modifying agents is a significant advance over separation technologies that rely on separation or detection of targets using beads coated with antibodies that are immunoreactive with a particular target. Not only are the simple modifying agents disclosed herein cheaper and easier to produce than antibody coated beads, but they are also of more general applicability and do not require identification and production of antibodies immunoreactive with each and every possible target of interest. The need for such extensive information of possible targets is a significant limitation to the general applicability and cost effectiveness of previously available technologies.

The target can be DNA, RNA, protein, bacterial cells or spores, viruses, small organic molecules, or chemical compounds. Furthermore, target DNA, RNA, or protein can be derived from human or non-human animals, plants, bacteria, viruses, fungi, or protozoa. The invention contemplates the use of this method alone or in combination with the previously disclosed SNAP methodology for separating and analyzing nucleic acids under conditions which inhibit the degradation of the nucleic acid or the contamination of the nucleic acid sample with agents that inhibit the further analysis of the target nucleic acid.

Following separation of target using either methodology, the target can be further analyzed using routine techniques in cell biology, molecular biology, chemistry, or toxicology. The particular technique can be selected based on the target, and one of skill in the art can readily select an appropriate technique(s). In one embodiment, the target is DNA obtained from a particular biological or environmental sample, and further analysis of the DNA may involve PCR analysis of the DNA. The DNA may be of human, animal, bacterial, plant, fungal, protozoan, or viral origin depending on the particular application of the technology. In another embodiment, the target is RNA obtained from a particular biological or environmental sample, and further analysis of the RNA may involve RT-PCR analysis of the RNA or in situ hybridization analysis of RNA. The RNA may be of human, animal, bacterial, plant, fungal, protozoan, or viral origin. In still another embodiment, the target is a bacterial cell or spore obtained from a particular biological or environmental sample. Further analysis may involve analysis of the bacterial cell or spore itself. Exemplary methods for analyzing the cells or spores include, but are not limited to, microscopy, culture, cytological testing, and the analysis of bacterial cell surface markers. Additionally, analysis of the target bacterial cell or spore may involve analysis of DNA or RNA prepared from the target cell or spore, as well as analysis of both the cell or spore itself and DNA or RNA prepared from the target cell or spore. In yet another embodiment, the target is a protein obtained from a particular biological or environmental sample. The protein may be of human, animal, bacterial, plant, fungal, protozoan, or viral origin depending on the particular application of the technology. Further analysis of the protein may involve peptide sequencing, mass spectroscopy, and 1 or 2-dimensional gel electrophoresis.

In a second aspect, the present invention provides particular surface modifying agents that can be coupled to the surface of a substrate. Substrates modified with one or more surface modifying agents have an increased affinity for particular targets in comparison to either unmodified substrates or substrates modified with other surface modifying agents. The invention contemplates modification of a wide range of substrates including, but not limited to plates, chips, coverslips, culture vessels, tubes, beads, probes, fiber-optics, filters, cartridges, strips, and the like. Furthermore, the invention contemplates that such substrates can be composed of any of a wide range of materials including, but not limited to, plastic, glass, metal, and silica, and furthermore that the materials may possess magnetic or paramagnetic characteristics. As can be construed from the list of exemplary substrates, a suitable substrate can be virtually any size or shape, and one of skill in the art can readily select a suitable substrate based on the particular target as well as the particular materials from which the target must be analyzed.

In one embodiment, a substrate is modified with one surface modifying agent. In another embodiment, a substrate is modified with two or more surface modifying agents. In still another embodiment, the surface modifying agent is coupled to the substrate via a cleavable linker which allows the release of the modifying agent from the substrate. When multiple surface modifying agents are used, the agents may each have an increased affinity for the same target, or the agents may have an increased affinity for different targets so that the modified substrates are capable of separating more than one target. Furthermore, when multiple surface modifying agents are used, the agents may each have the same affinity for a particular target or the agents may have varying affinities for a particular target.

In a third aspect, the present invention provides apparatuses which can be used to separate targets from biological, chemical or environmental samples. The invention includes two classes of apparatuses. The first class includes apparatuses which facilitate the interaction between substrates and samples. Such apparatuses are particularly important for large scale implementation of the methods of the present invention. By way of example, when separating targets from small samples of soil, water, air, or bodily fluids, the efficient delivery of modified substrate to the sample containing the target is straightforward. In such settings, it is relatively easy to insure that the entire sample is contacted with substrate, and thus the substrate has an opportunity to interact with target throughout the entire sample. However, when larger samples are involved, it is a less straightforward process to ensure that the substrate contacts target which may be distributed evenly or unevenly throughout the large sample. For such applications, the invention provides a device for facilitating the even mixing of substrate throughout large samples containing target. One example which illustrates an application of this apparatus is in industrial food-processing facilities. Large vessels containing food, beverage, or ingredients for the production of various foods or beverages may become contaminated with bacteria, viruses, or chemicals during processing or storage. However, the efficient detection of such potentially harmful contaminants may be hindered by the large volumes of sample. One application of this first class of apparatus is in the food-processing industry where the apparatus could be used to regularly and efficiently evaluate the quality of large volumes of food or ingredients.

The second class of apparatuses provides alternative coated substrates, such as filters and cartridges, which can be used to readily process a sample containing a target. These apparatuses have a wide range of biological, environmental, and industrial applications, and can be used to efficiently analyze solid, liquid, or gaseous samples. Of particular note, filters and cartridges which analyze sample based on the Affinity Protocol can be used alone or can be used in combination with other available filters and cartridges. Filters and cartridges can be used in any of a variety of settings.

Of particular note, the methods, compositions, and apparatuses of the present invention can be used in a traditional laboratory or hospital setting, or in the field where access to other laboratory equipment and supplies may be limited. Furthermore, using the compositions and apparatuses of the present invention, the separation methods can be performed in less time than other traditional separation methodologies. The ability to perform rapid analysis of samples is crucial in any of a number of laboratory and field applications. By way of example, decreased sample analysis time can allow doctors and hospitals to provide immediately to patients the results of diagnostic tests. This shortens the time prior to which treatment can begin and decreases the risk of patient flight and noncompliance. By way of further example, rapid analysis facilitates crime scene investigations. By way of still further analysis, rapid analysis of environmental pollution facilitates correlating the pollution with particular industrial or natural events.

In any of the foregoing, the separation methods of the present invention (whether implemented using filters, cartridges, or other substrates) can be performed in less than 30 minutes. In another embodiment, the separation methods can be performed in less than or equal to 25, 20, 15, 14, 13, 12, 11, 10, 9, or 8 minutes. In yet another embodiment, the separation methods can be performed in less than or equal to 7, 6, 5, or 4 minutes. Targets separated using the methods of the present invention can, optionally, be further analyzed using other rapid analytical techniques.

In any of the foregoing, the time required to carry out the separation methods of the present invention (whether implemented using filters, cartridges, or other substrates) includes the time required for binding of target to substrate (e.g., capture time) and may also include the time required to release the target from the substrate (e.g., elution time). In one embodiment, the capture time can be less than or equal to 30, 25, 20, 15, 14, 13, 12, 11, 10, 9, or 8 minutes. In another embodiment, the capture time can be less than or equal to 7, 6, 5, 4, 3, 2, or 1 minutes. In another embodiment, the capture time can be 5-10 minutes, 1-5 minutes, 1 minute, or less than 1 minute. Targets captured by the methods of the present invention can, optionally, be eluted from the substrate. Eluted targets can, optionally, be further analyzed using other rapid analytical techniques.

In another embodiment, the elution time can be less than or equal to 30, 25, 20, 15, 14, 13, 12, 11, 10, 9, or 8 minutes. In another embodiment, the elution time can be less than or equal to 7, 6, 5, 4, 3, 2, or 1 minutes. In another embodiment, the elution time can be 5-10 minutes, 1-5 minutes, 1 minute, or less than 1 minute. Targets eluted by the methods of the present invention can, optionally, be further analyzed using other rapid analytical techniques.

In any of the foregoing, the separation methods of the present invention may require the use of an effective amount of a substrate. Although the use of a larger concentration of substrate may be advantageous in certain applications, the use of a minimal concentration of substrate helps reduce the cost of the method and helps increase its ease of use in the field (e.g., reduces the amount of consumable reagents required for use). In one embodiment, the amount of substrate is greater than 10 mg/mL of sample. In one embodiment, the amount of substrate is less than or equal to 10 mg/mL of sample. In another embodiment, the amount of substrate is less than or equal or 7, 6, or 5 mg/mL of sample. In still another embodiment, the amount of substrate is less than or equal to 4, 3, 2, or 1 mg/mL of sample. In still another example, the amount of substrate is 5-10 mg/ml of sample or 1-5 mg/mL of sample.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims.

### DETAILED DESCRIPTION OF THE DRAWINGS

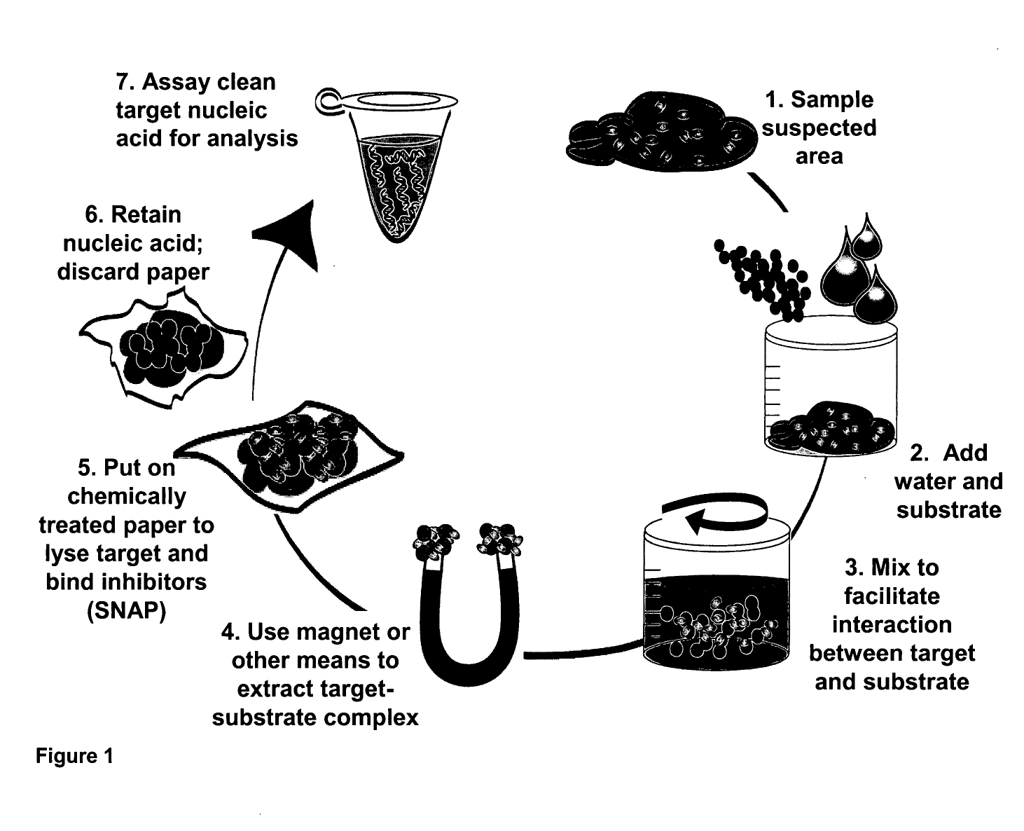

FIG. 1 provides a schematic representation of the Affinity Protocol.

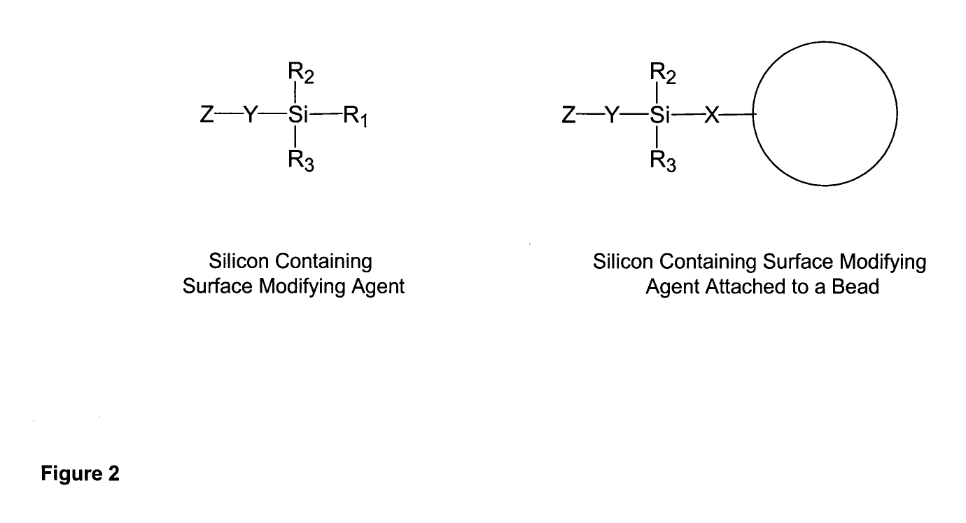

FIG. 2 shows a representative silicon containing surface modifying agent (left drawing) and a substrate modified with the silicon containing surface modifying agent (right drawing).

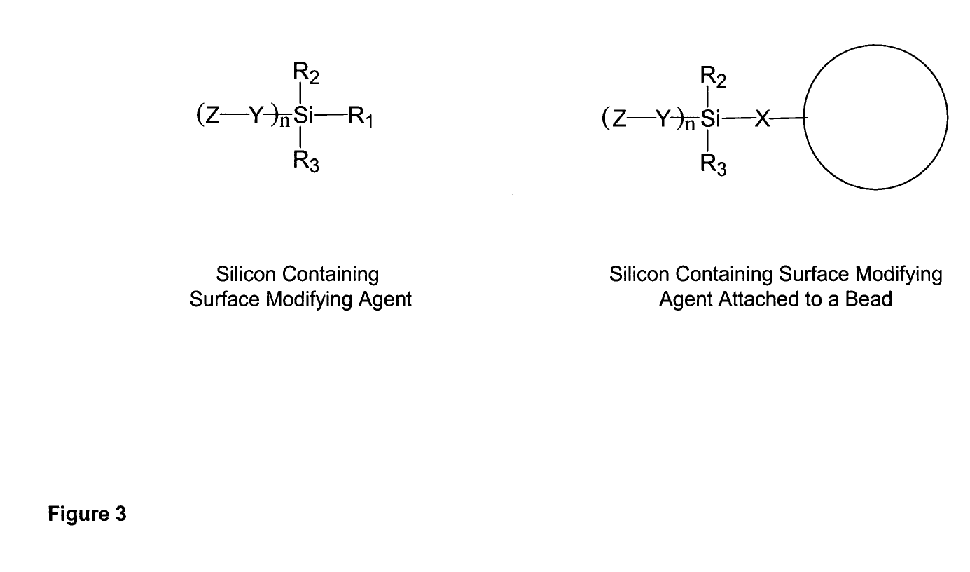

FIG. 3 shows a representative silicon containing surface modifying agent (left drawing) and a substrate modified with the silicon containing surface modifying agent (right drawing). In contrast to the surface modifying agent represented in FIG. 2, this model provides surface modifying agents containing multiple active regions which may be the same or different from each other.

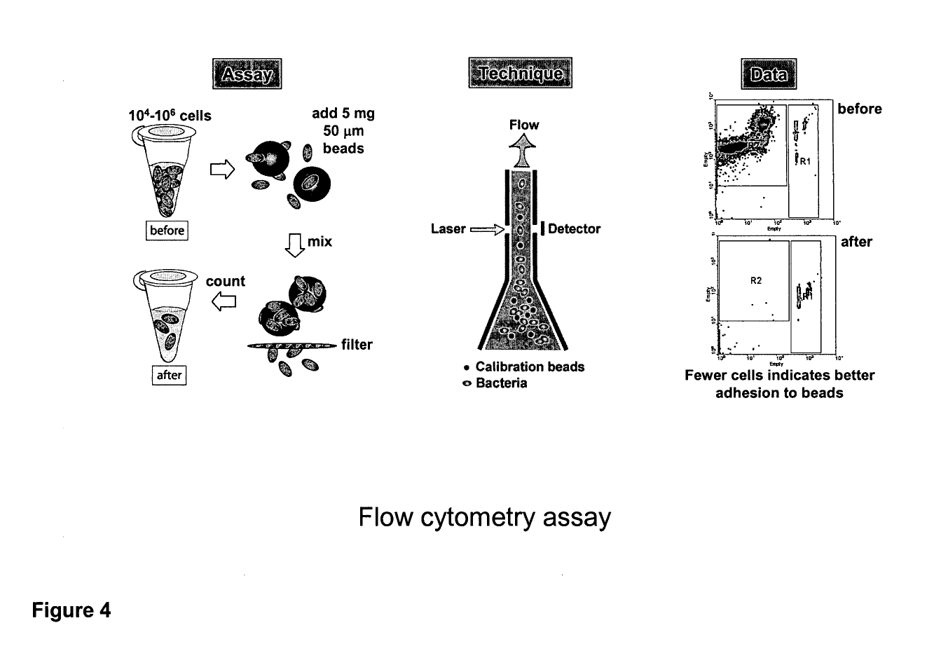

FIG. 4 shows a flow cytometry assay which can be used to readily assess and quantify the interaction between a substrate and a target.

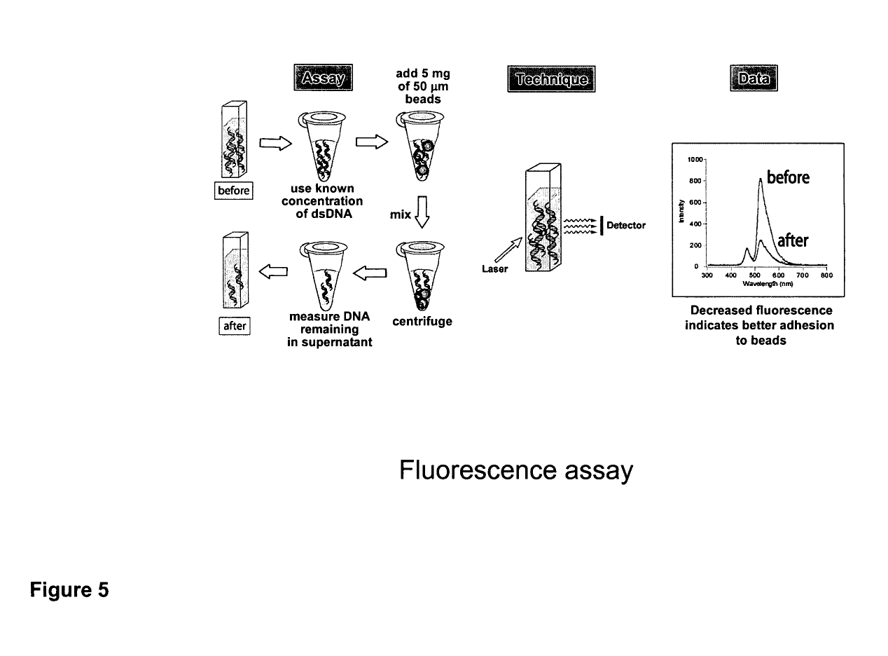

FIG. 5 shows a fluorescence assay which can be used to readily assess and quantify the interaction between a substrate and a target.

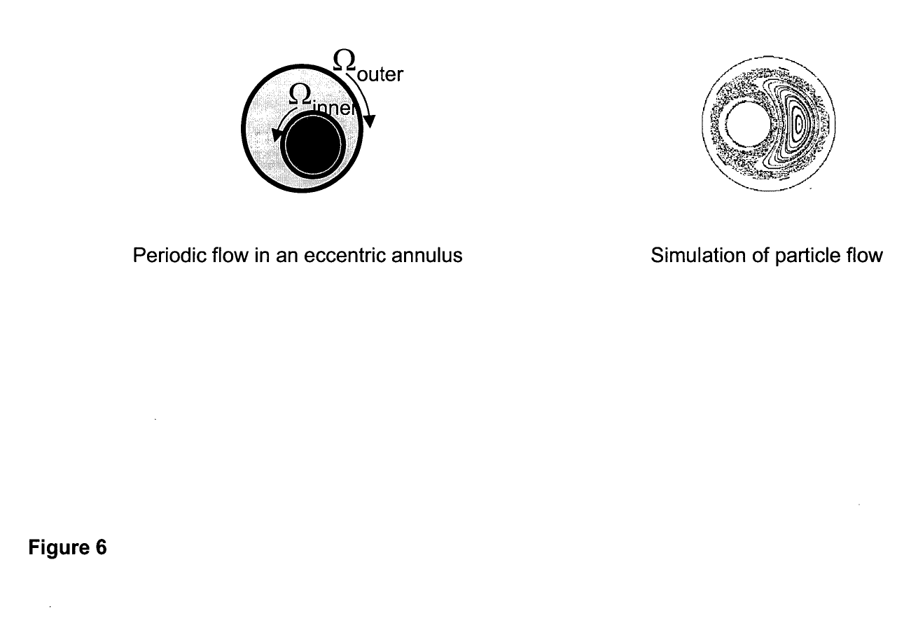

FIG. 6 illustrates the principle of journal bearing flow. The schematic at the right shows the results of a simulation of journal bearing flow used to mix a particulate slurry.

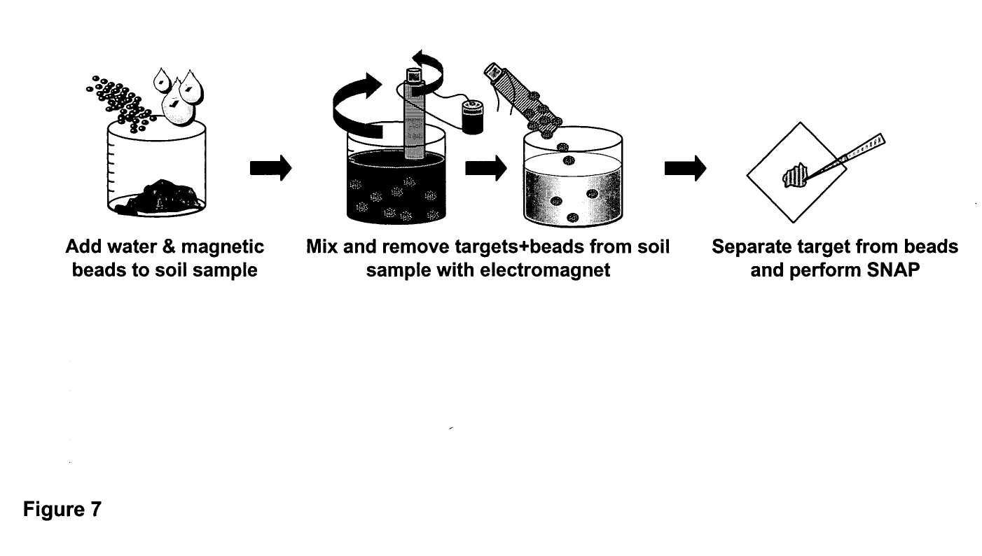

FIG. 7 shows a schematic depicting the Large-Scale Affinity Protocol. The large scale protocol involves the use of a Chaotic mixing device to facilitate the interaction between substrate and target in the standard affinity protocol. In this schematic representation, the substrate (magnetic beads), the sample soil, and water are mixed to create a slurry. The slurry, which contains the target and substrate, is placed in the Chaotic mixing device and mixed at low speed to facilitate interaction between the target and substrate. Following mixing, the inner cylinder is replaced by an electromagnet which is used to remove the target-substrate complexes. Since the substrate was magnetic beads, the target-substrate complexes are readily attracted to the electromagnet. Following removal of the target-substrate complexes from the slurry, the target cells are separated from the beads, and then lysed and processed using SNAP to examine DNA contained within the target cells.

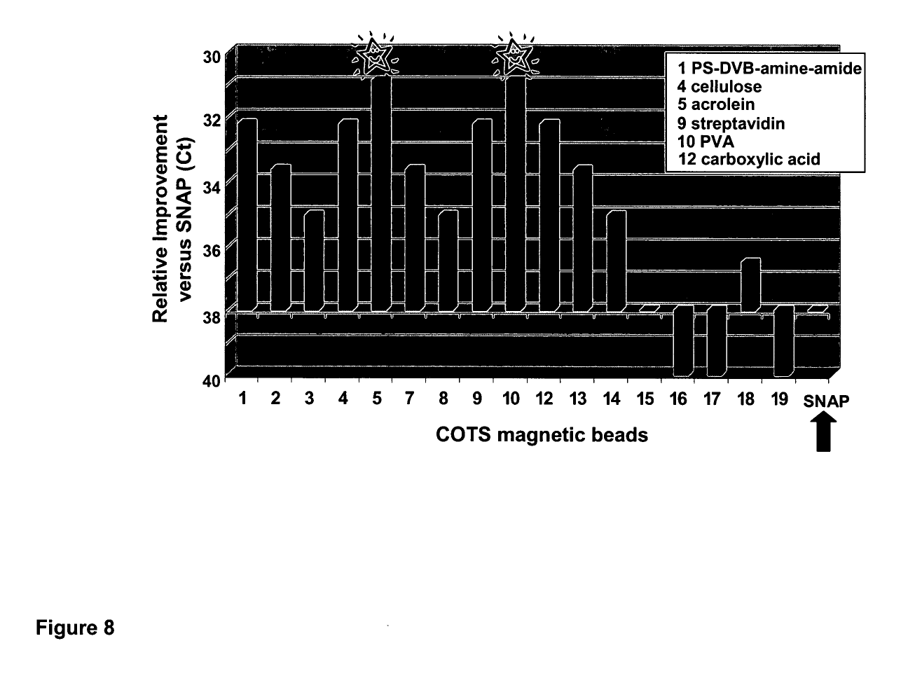

FIG. 8 summarizes the results of analysis of commercially available magnetic beads. The data was normalized to the signal for samples analyzed by SNAP alone so that the graphical representation presented in the figure demonstrates which beads enhanced signal versus SNAP alone.

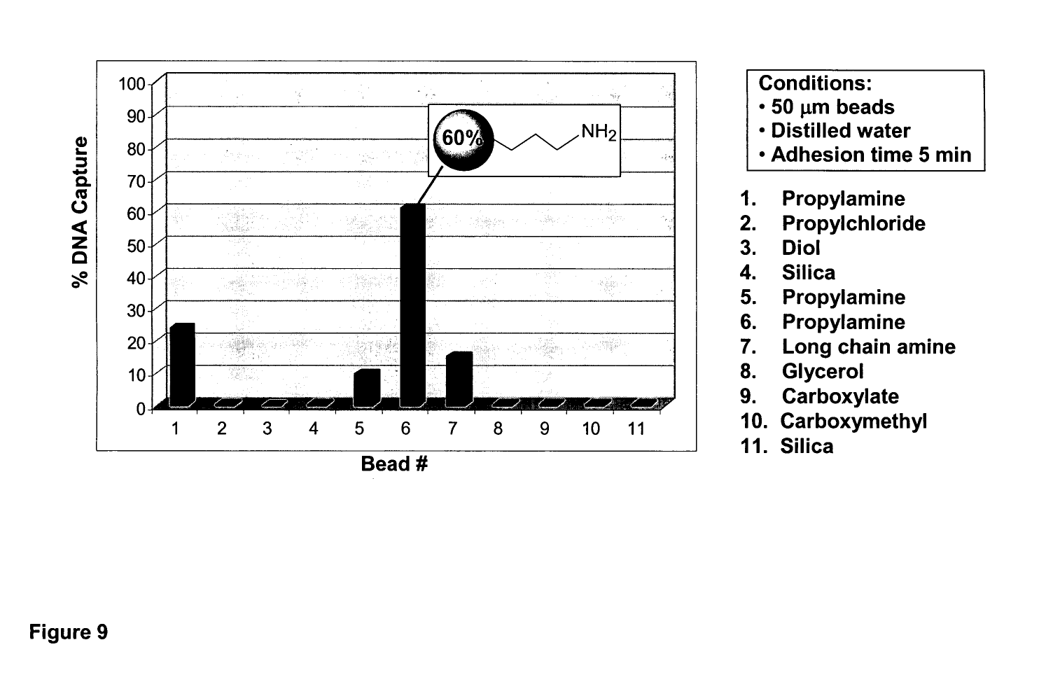

FIG. 9 summarizes the results of analysis of commercially available non-magnetic beads. The efficacy of these beads was assessed by measuring the percentage of DNA that adhered to the bead following incubation of the bead with a sample.

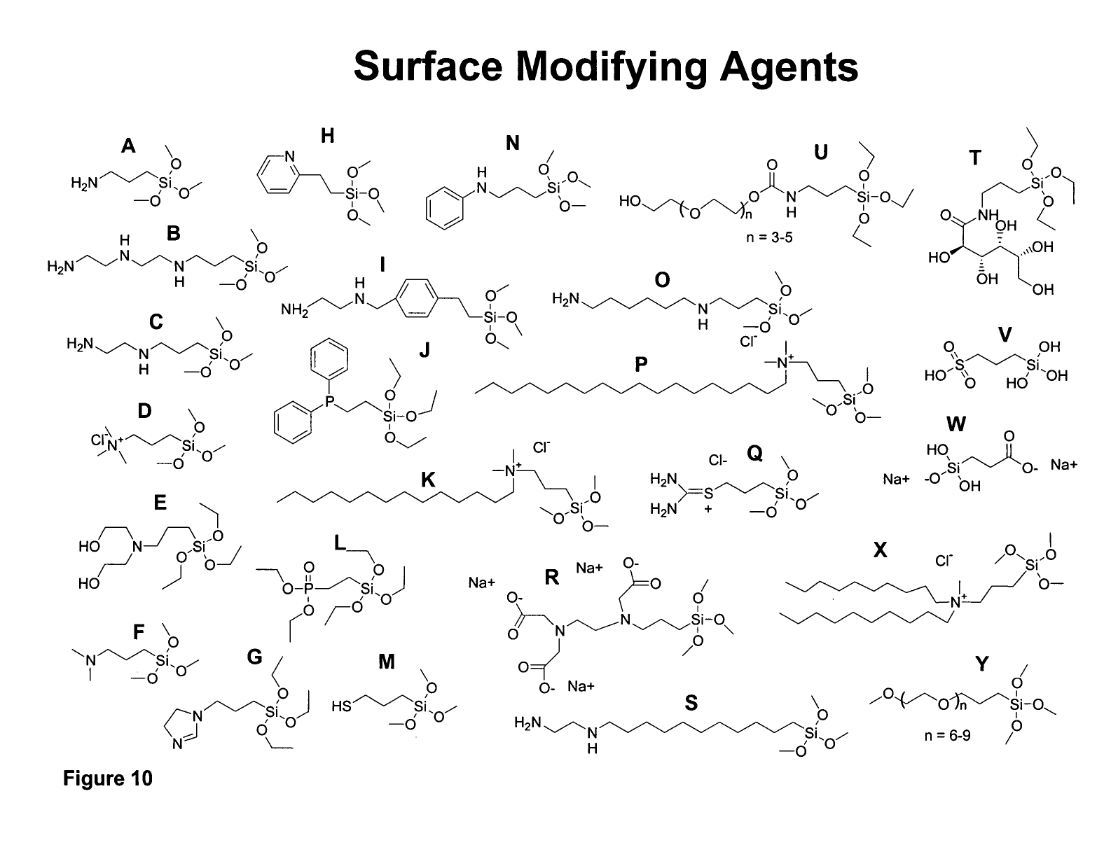

FIG. 10 shows the structure of the surface modifying agents (lettered A-Y) used to modify the surface of several different substrates.

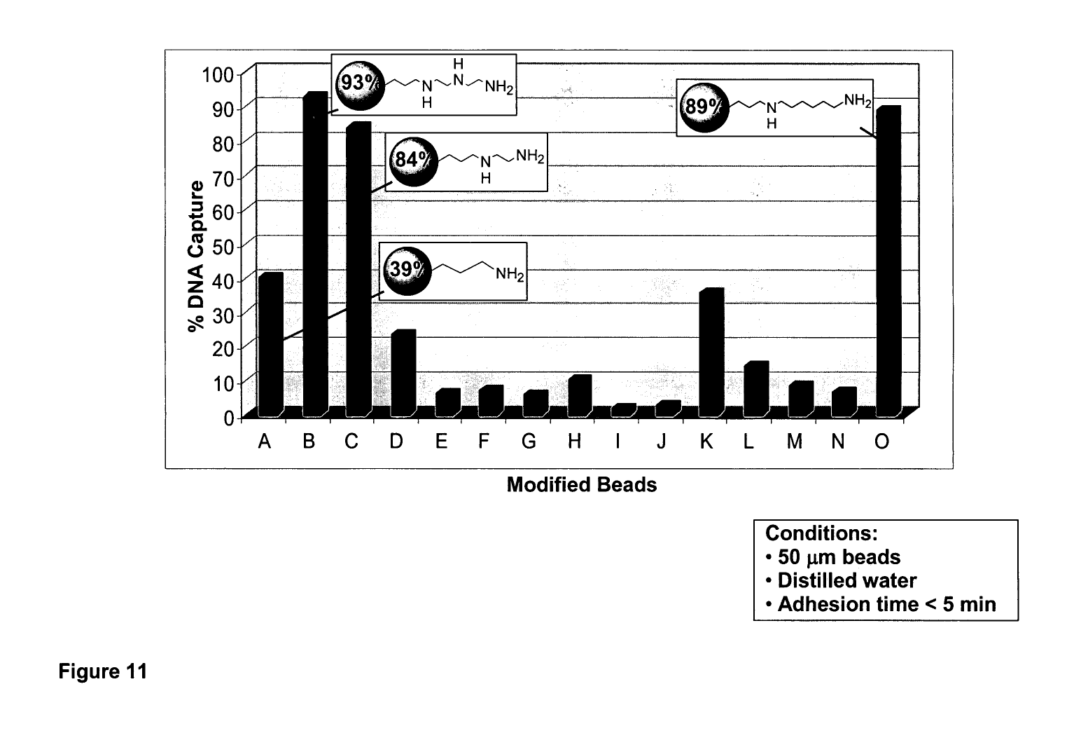

FIG. 11 shows that several of our amine-functionalized beads have improved adhesion for DNA.

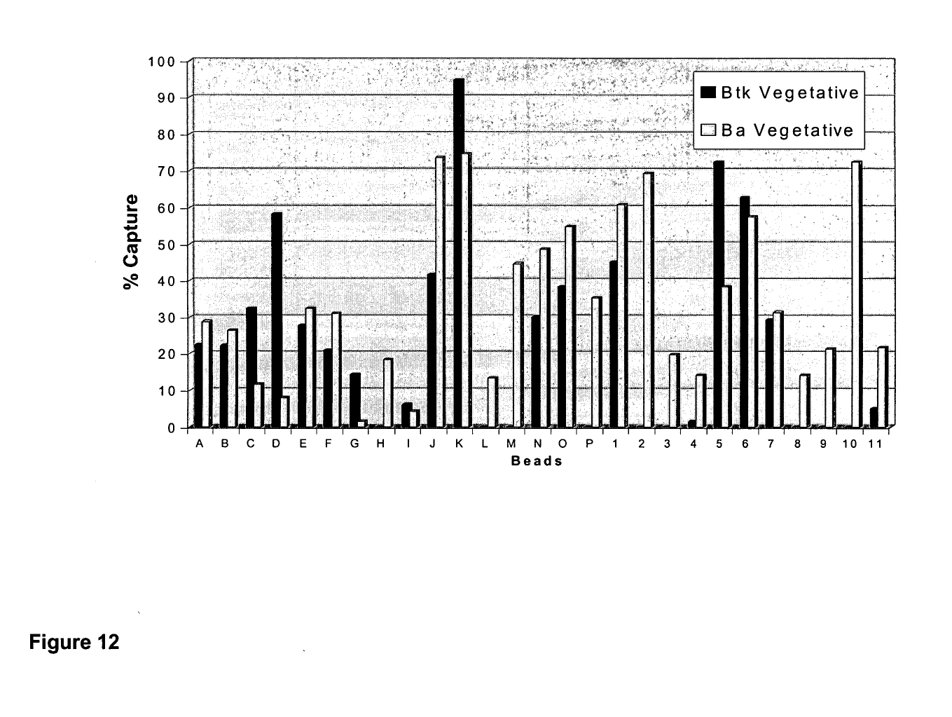

FIG. 12 shows the adhesion of both our amine-functionalized beads and several commercially available beads to two different bacterial targets.

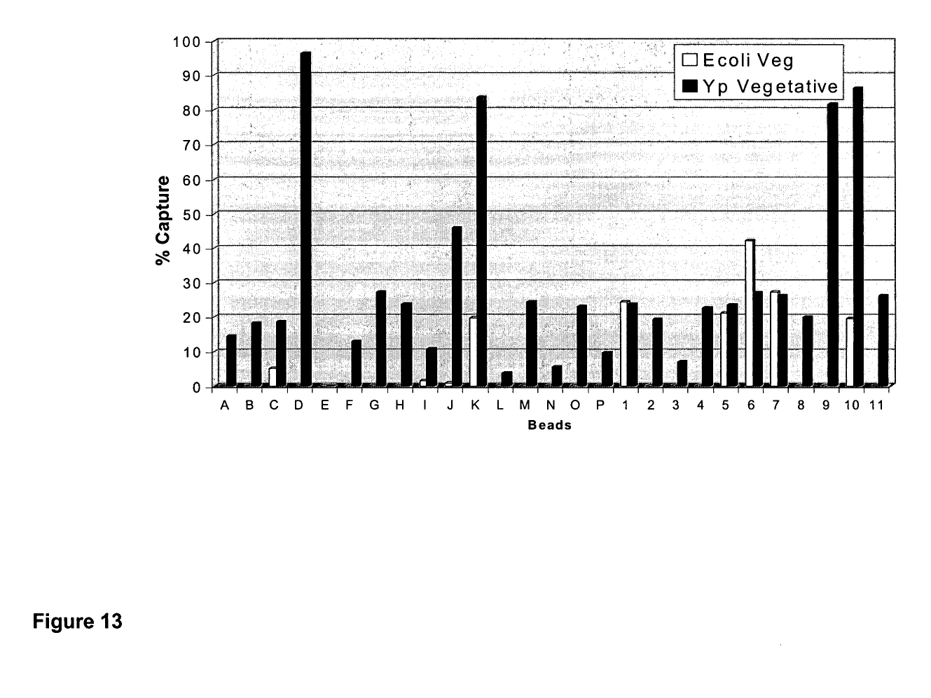

FIG. 13 shows the adhesion of both our amine-functionalized beads and several commercially available beads to two different bacterial targets.

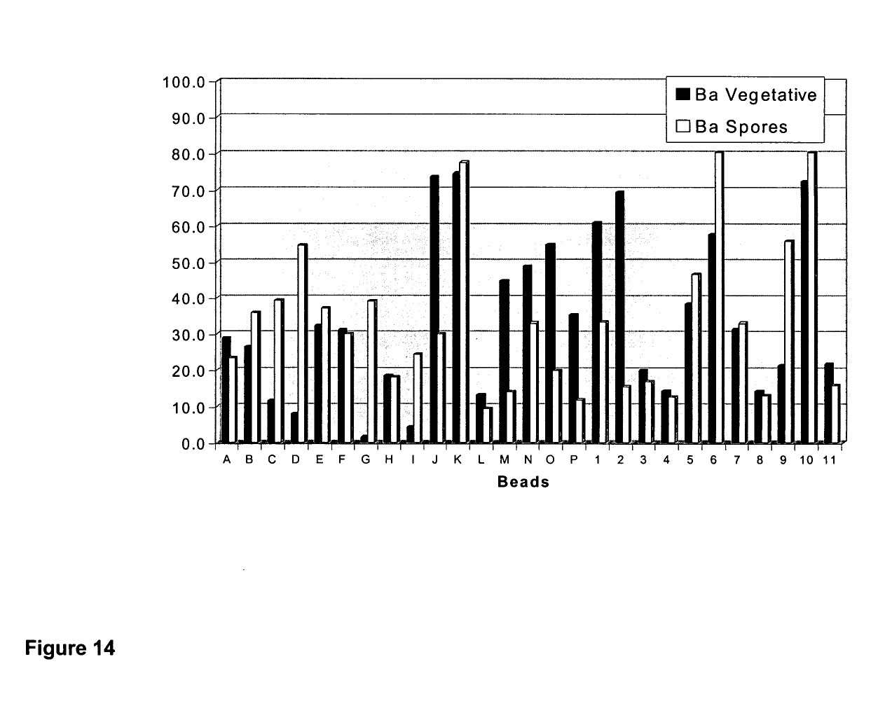

FIG. 14 shows the adhesion of both our amine-functionalized beads and several commercially available beads to the vegetative versus the sporulated form of a bacterial target.

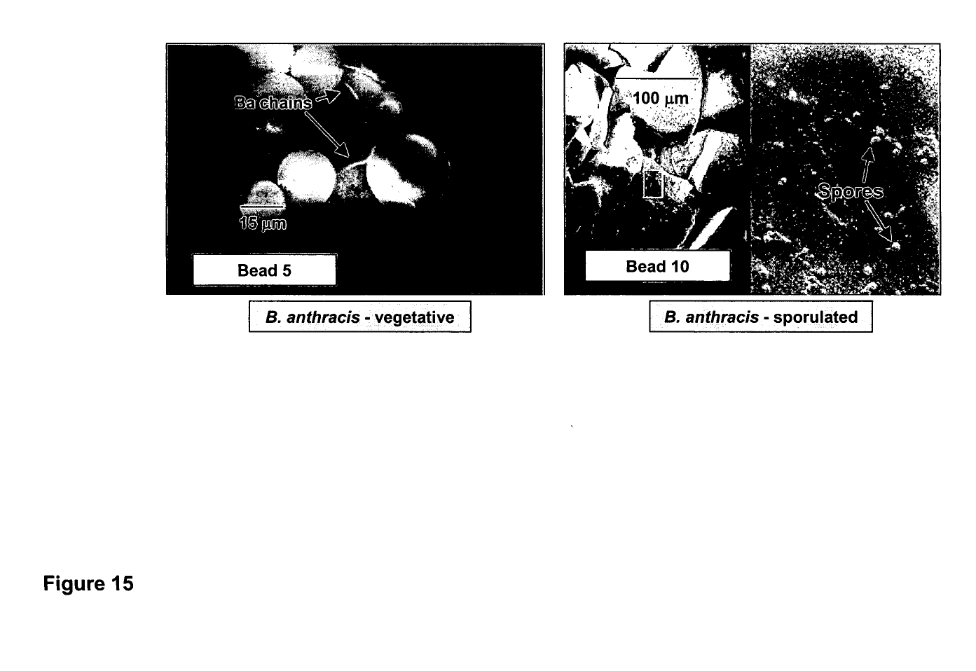

FIG. 15 shows SEM images of bacterial targets physically adhered to the surface of various substrates.

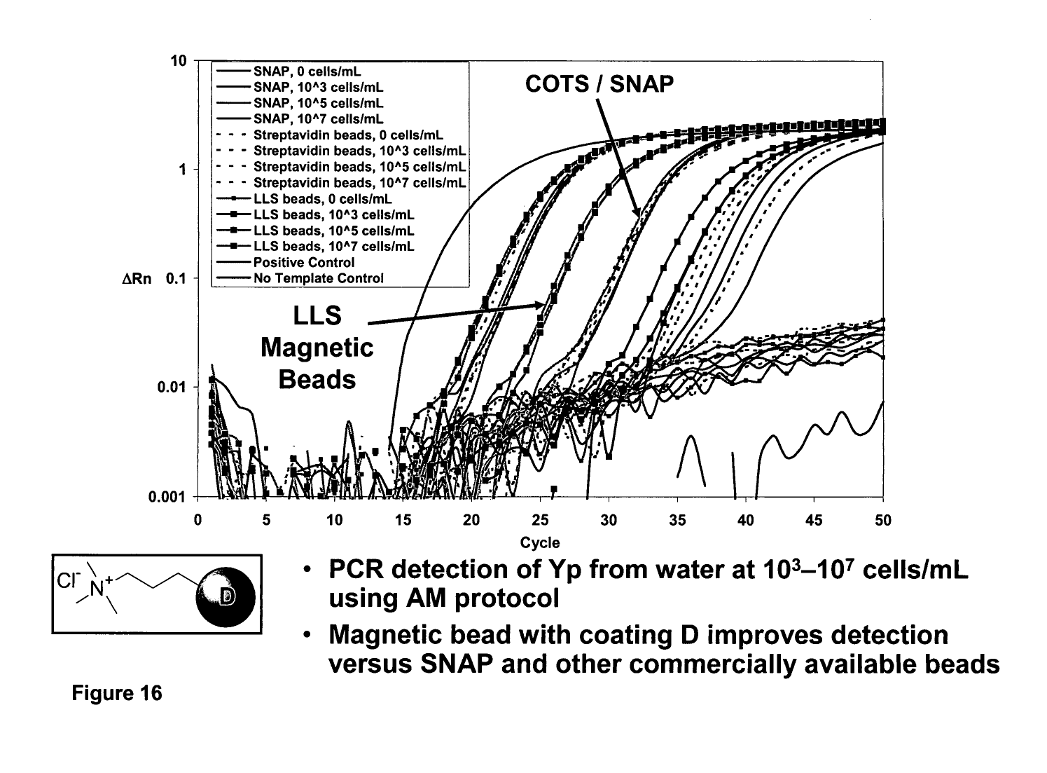

FIG. 16 shows that identification of target (in this case bacterial DNA) is improved using a combination of the Affinity Protocol and SNAP.

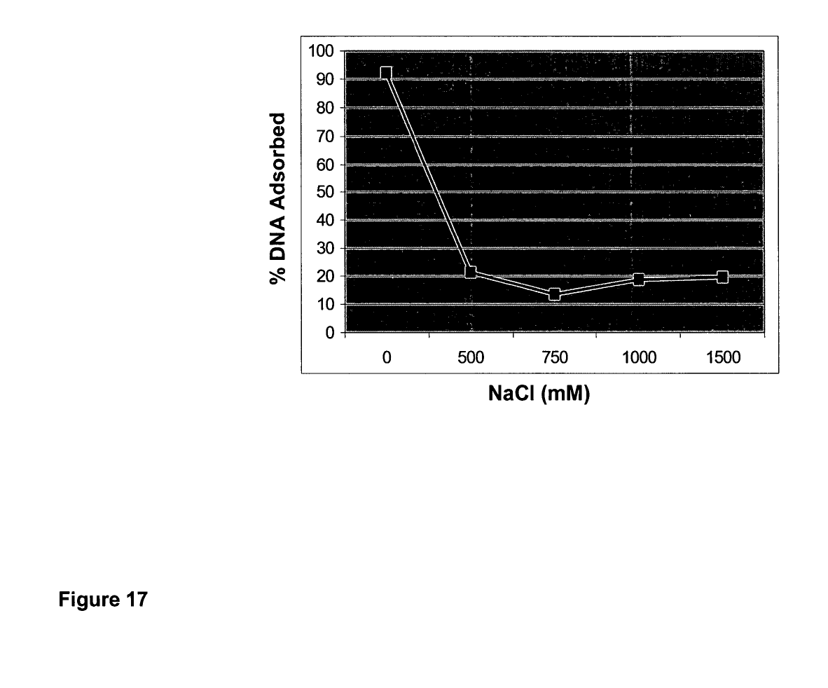

FIG. 17 shows that the adhesion of DNA to a coated substrate is influence by the salt concentration.

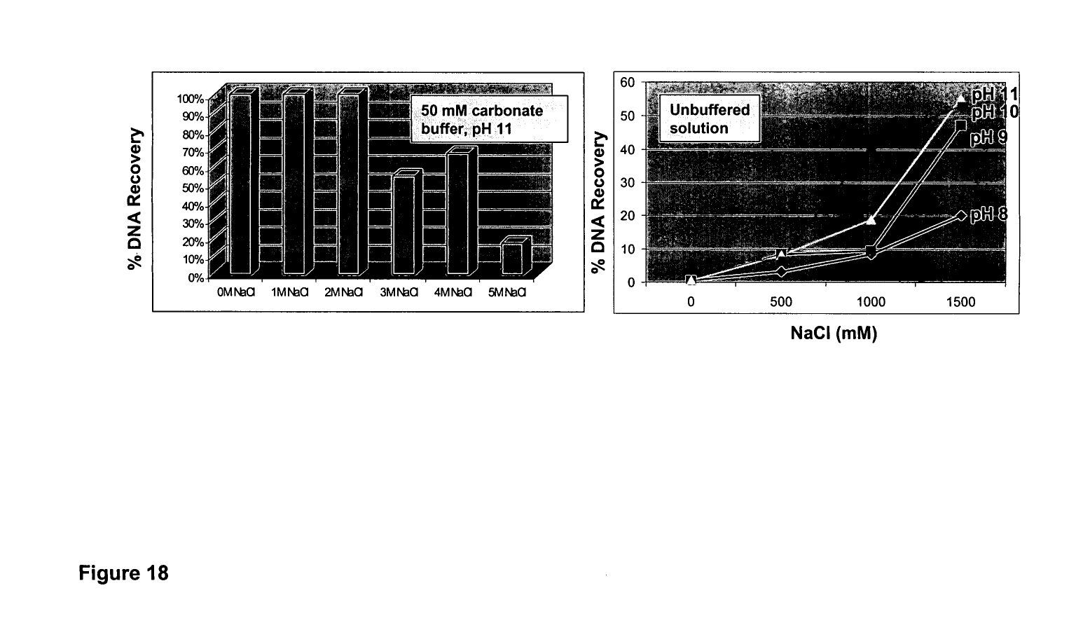

FIG. 18 shows that the adhesion of DNA to a coated substrate is influence by both the salt concentration and the pH.

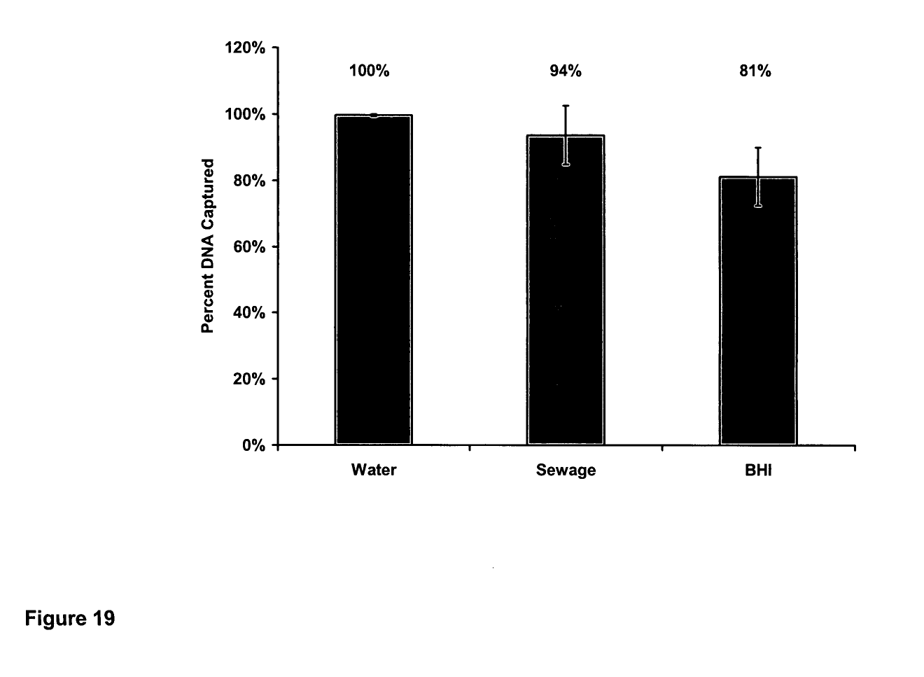

FIG. 19 shows that substrates can efficiently bind target DNA present in a variety of samples including water, culture medium, and non-laboratory-grade environmental water.

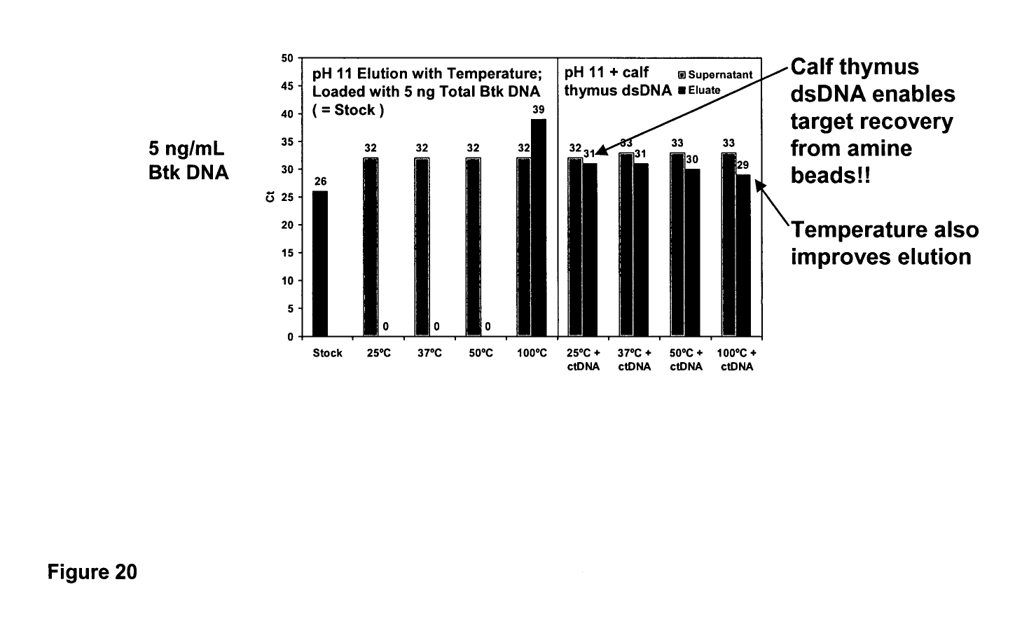

FIG. 20 shows that the manipulation of temperature can be used to elute target DNA from a substrate.

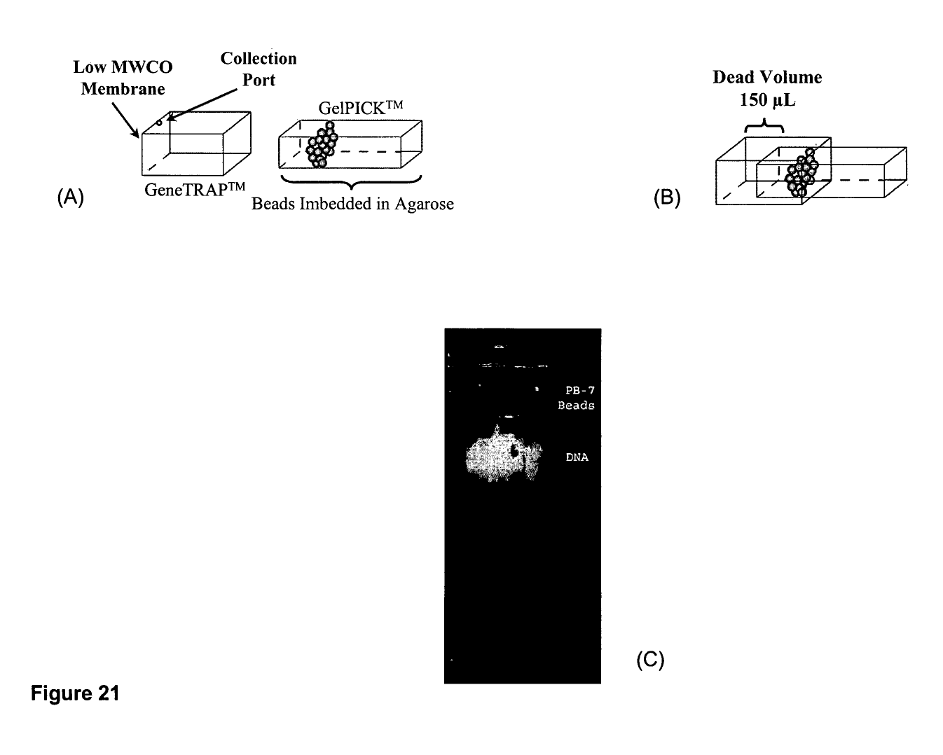

FIG. 21 shows that target can be released from substrate using electroelution. FIG. 21A shows a diagram of the GeneCapsule apparatus and the placement of the substrate within the apparatus. FIG. 21B shows a diagram of the GeneCapsule apparatus following loading with substrate. FIG. 21C shows the elution of calf thymus DNA from amine beads following electroelution. Large quantities of calf thymus DNA can be seen migrating away from the substrate.

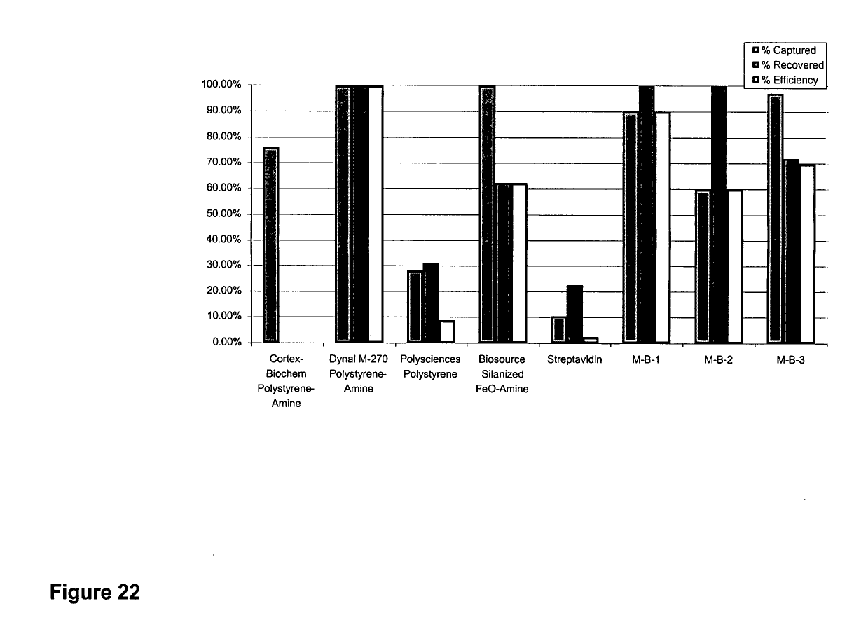

FIG. 22 shows a comparison of the capture and release activity of various magnetic beads with affinity for DNA. For each type of bead, one milligram of the substrate was added to 1 mL of 500 pg/mL DNA in standard deionized water. For each type of bead, the left most bar represents the percentage of DNA captured to the substrate. The middle bar represents the percentage of captured DNA released into an elution buffer including 150 μL of 100 μg/mL calf-thymus DNA in 0.01N NaOH. This is referred to as the percentage of recovered target and is the ratio of the recovered DNA to the captured DNA. The right-most bar represents the efficiency and is the ratio of recovered DNA to the total DNA (500 pg) present in the original sample.

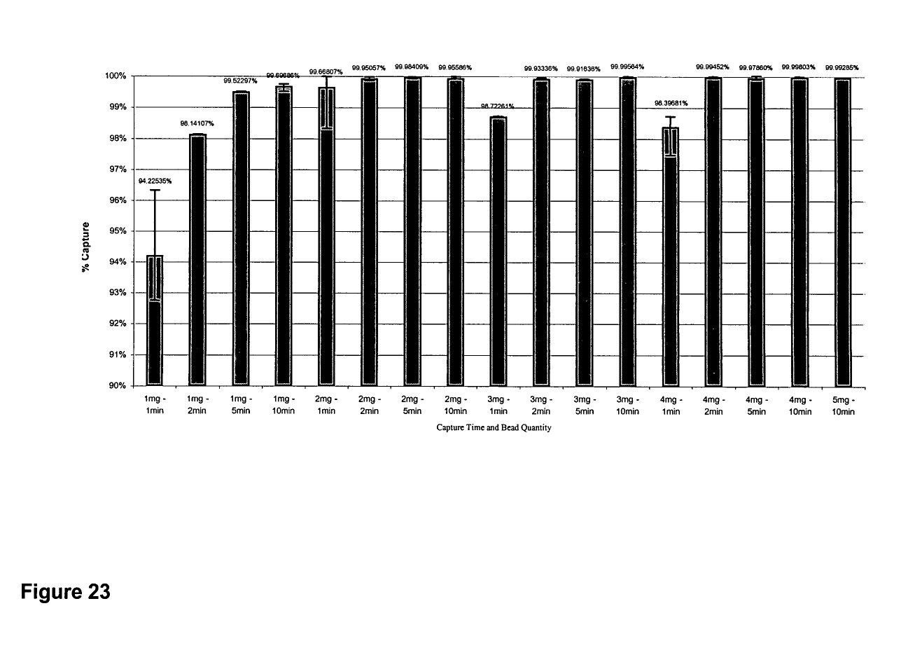

FIG. 23 shows the efficiency with which commercially available amine coated magnetic beads capture DNA as a function of substrate quantity and capture time (e.g., time of contact between substrate and sample).

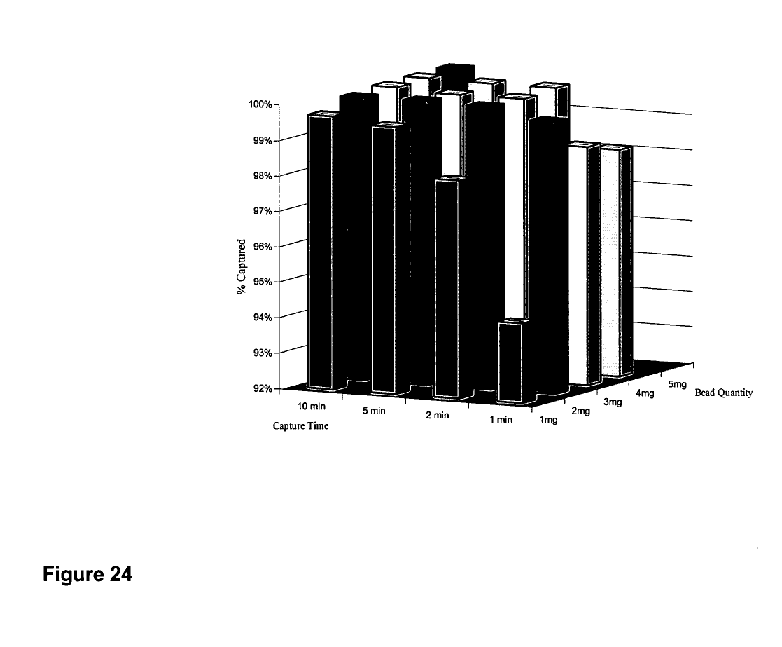

FIG. 24 shows the efficiency with which commercially available amine coated magnetic beads capture DNA as a function of substrate quantity and capture time (e.g., time of contact between substrate and sample).

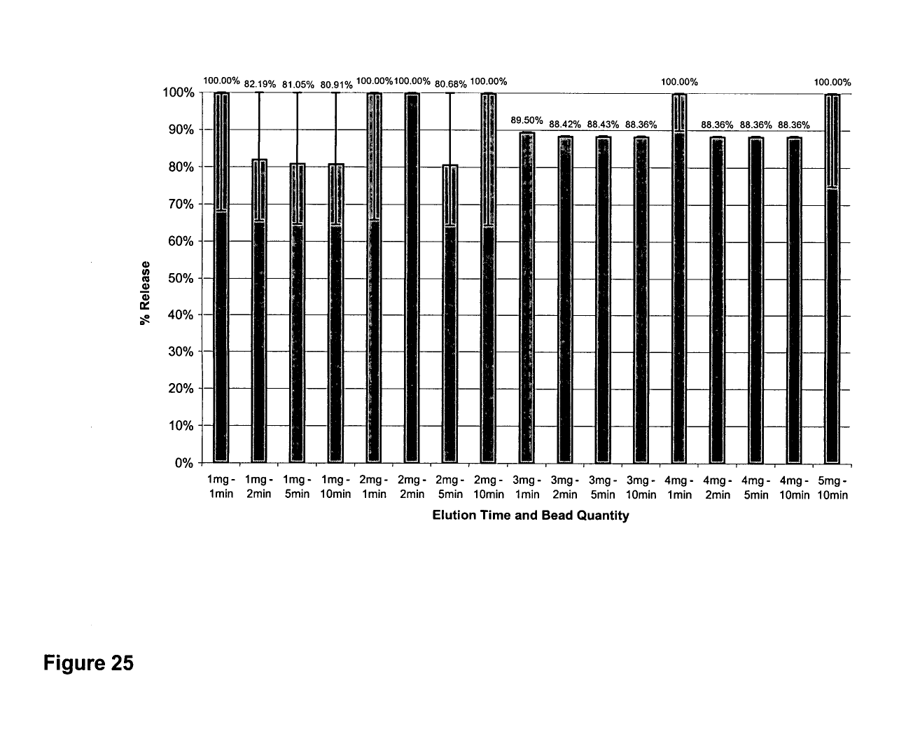

FIG. 25 shows the efficiency with which commercially available amine coated magnetic beads release DNA as a function of substrate quantity and elution time.

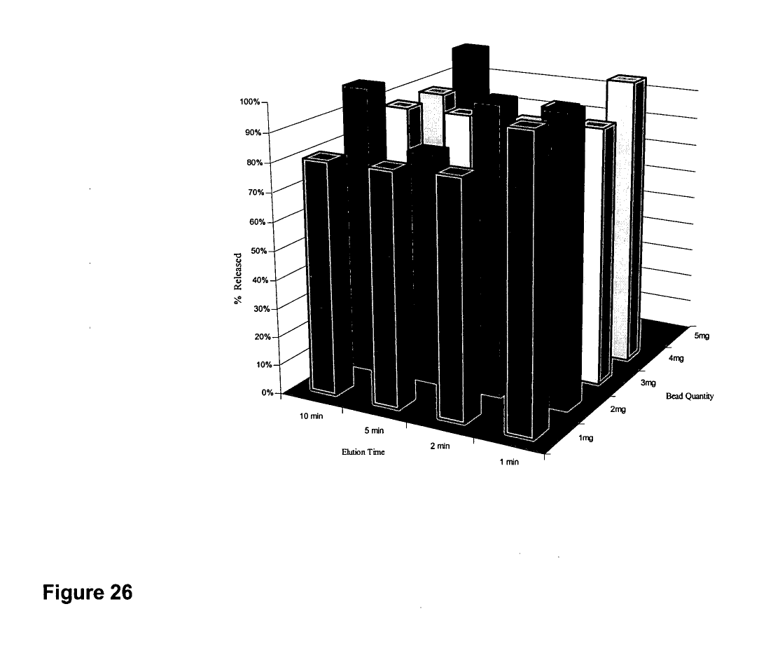

FIG. 26 shows the efficiency with which commercially available amine coated magnetic beads release DNA as a function of substrate quantity and elution time.

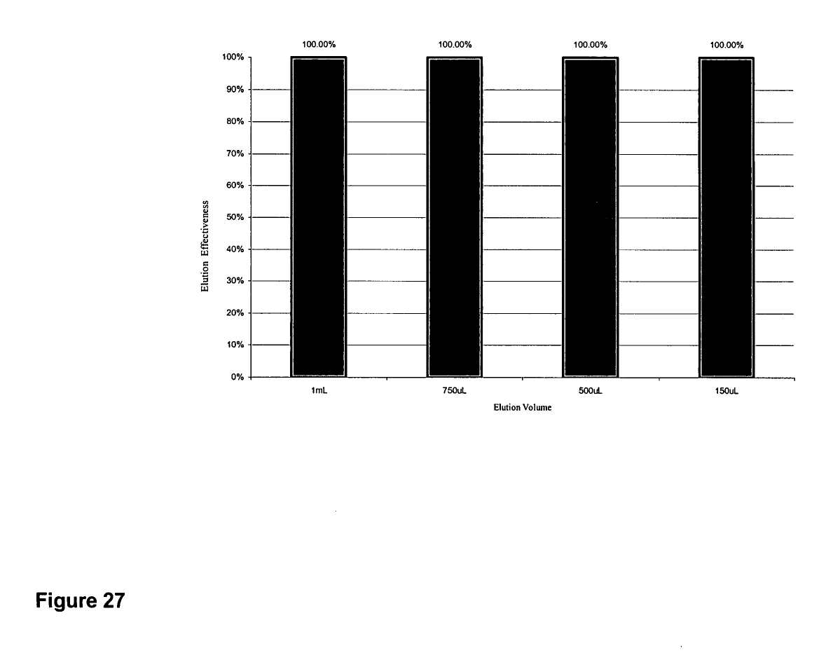

FIG. 27 shows the effect of elution volume on elution efficiency.

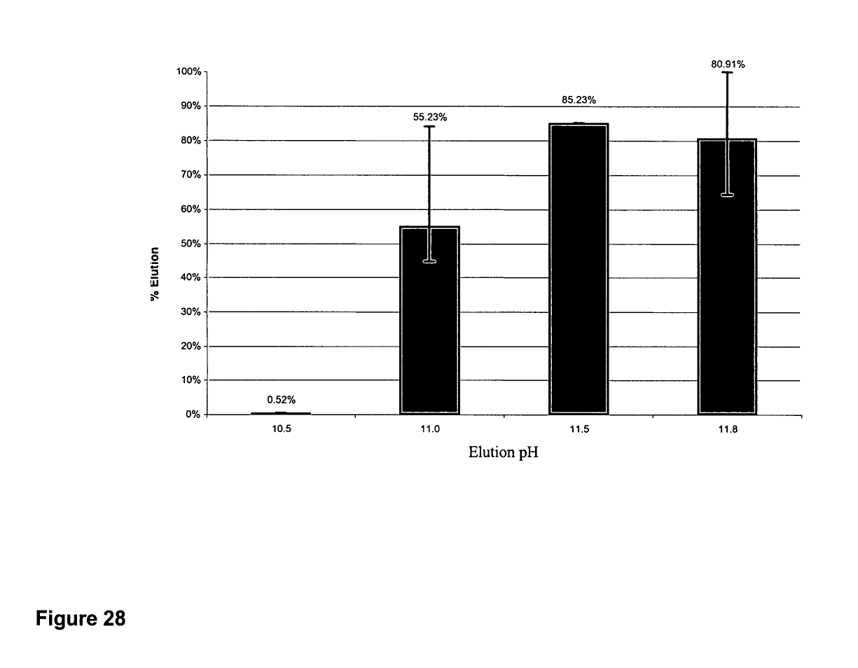

FIG. 28 shows the effect of pH on elution efficiency.

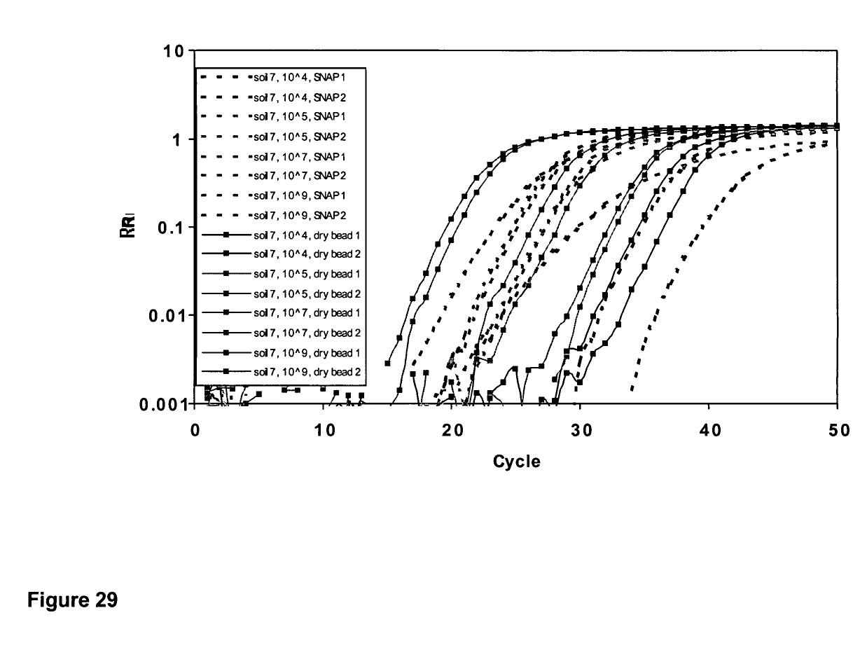

FIG. 29 shows PCR results following isolation of bacterial DNA from a dry soil sample using the dry Affinity Magnet protocol. The dashed lines indicate soil samples processed using only the SNAP method for isolating DNA, and the solid lines indicate soil samples that were contacted with electrostatically charged, non-magnetic beads prior to SNAP processing.

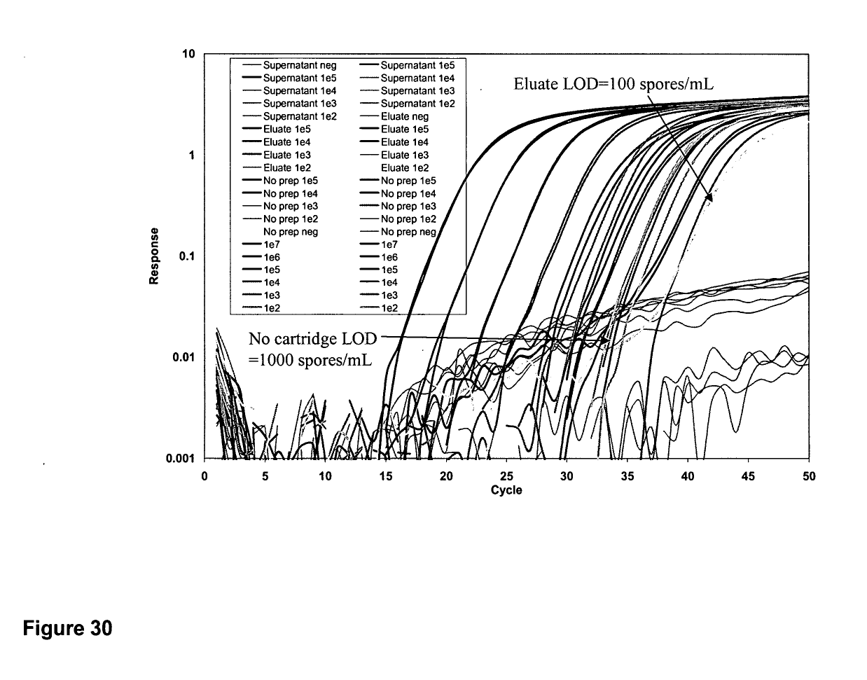

FIG. 30 shows PCR results following separation of bacterial spores from a sample composed of sand mixed with water to form a slurry, using a magnetic-bead-containing cartridge. DNA from target spores in sand was analyzed by PCR either directly or following separation from the sample using the Affinity Protocol. Separation of the target prior to PCR resulted in an increase in detection of one order of magnitude in comparison to direct PCR analysis of the target-containing sample.

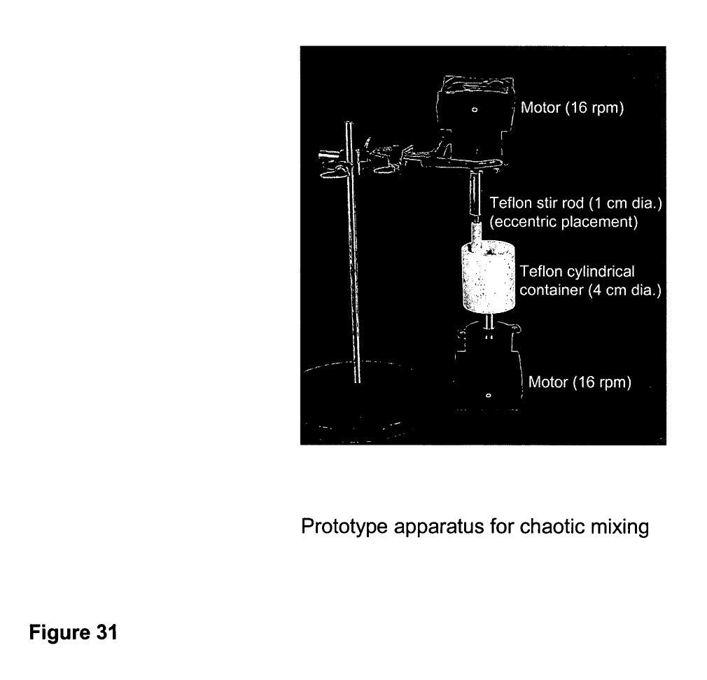

FIG. 31 shows an apparatus for chaotic mixing (A Chaotic Mixing Device).

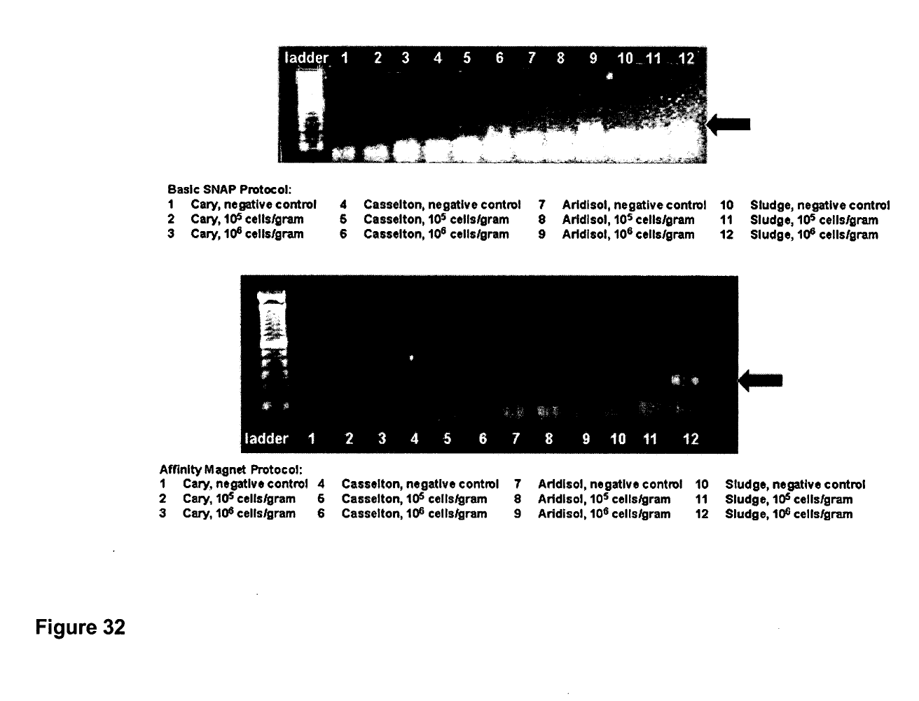

FIG. 32 shows gel electrophoresis of PCR reactions conducted on DNA isolated using either the SNAP protocol alone (top panel) or DNA isolated using the large-scale affinity protocol plus the SNAP protocol (bottom). In both panels, the arrow is used to indicate the amplified band. These results demonstrate that the large-scale affinity protocol improves the limits of detection in large samples.

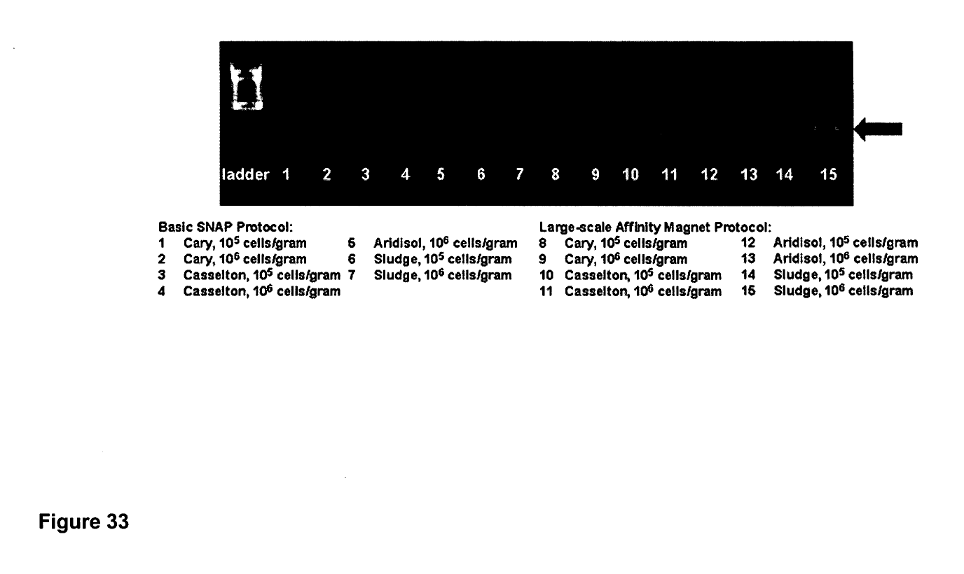

FIG. 33 shows gel electrophoresis of PCR reactions conducted on DNA isolated using either the SNAP protocol alone or DNA isolated using the large-scale affinity protocol plus the SNAP protocol. The arrow is used to indicate the amplified band. These results demonstrate that the large-scale affinity protocol improves the limits of detection in large samples.

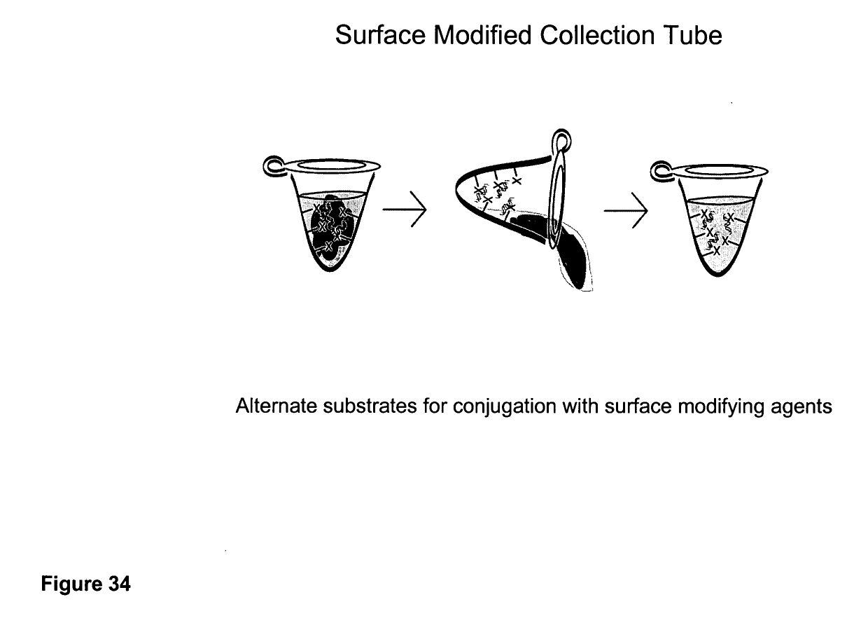

FIG. 34 shows a surface modified collection tube.

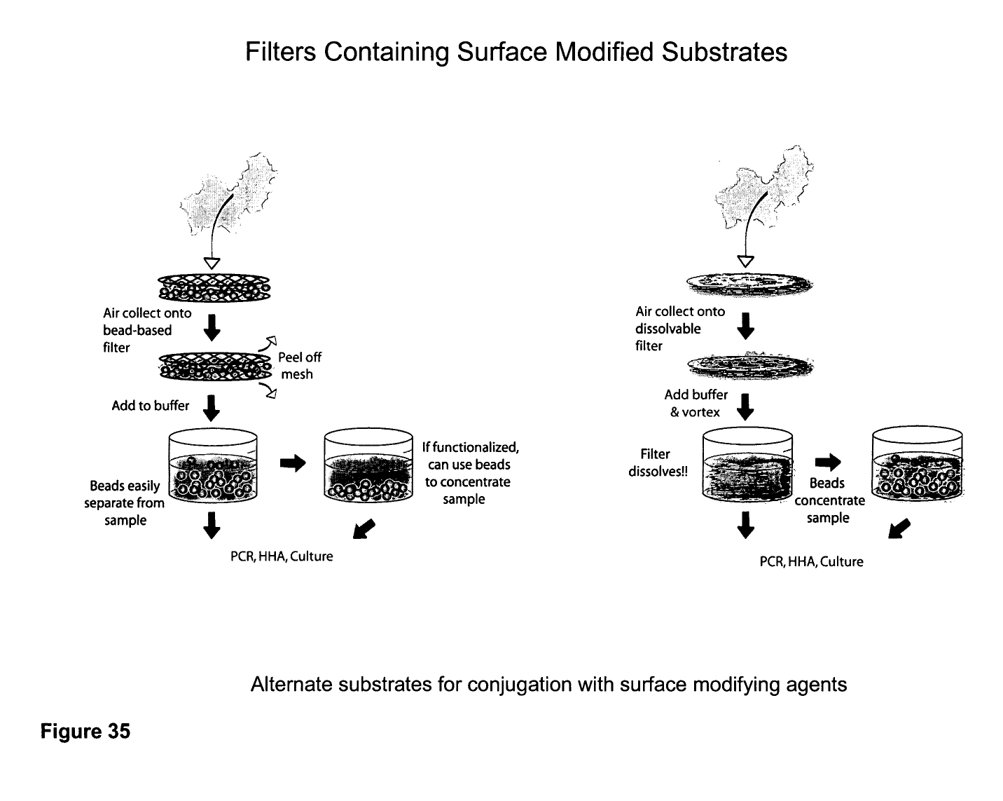

FIG. 35 shows two designs for filters containing surface modified substrates. Although the particular example provided in the figure indicates that the filters are used to collect air samples (gaseous sample), similar designs can be readily adapted for the construction of filters used to collect liquid samples.

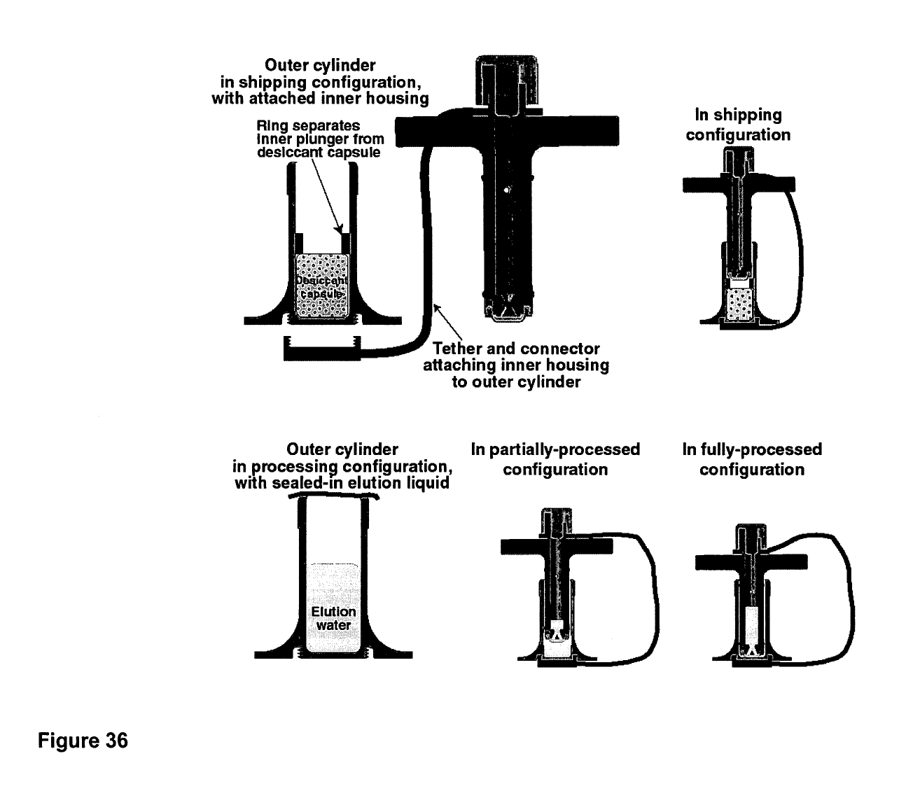

FIG. 36 shows a variant of the LiNK device that can be used to process a sample through one or more substrates. Additionally, the device helps preserve the sample after collection.

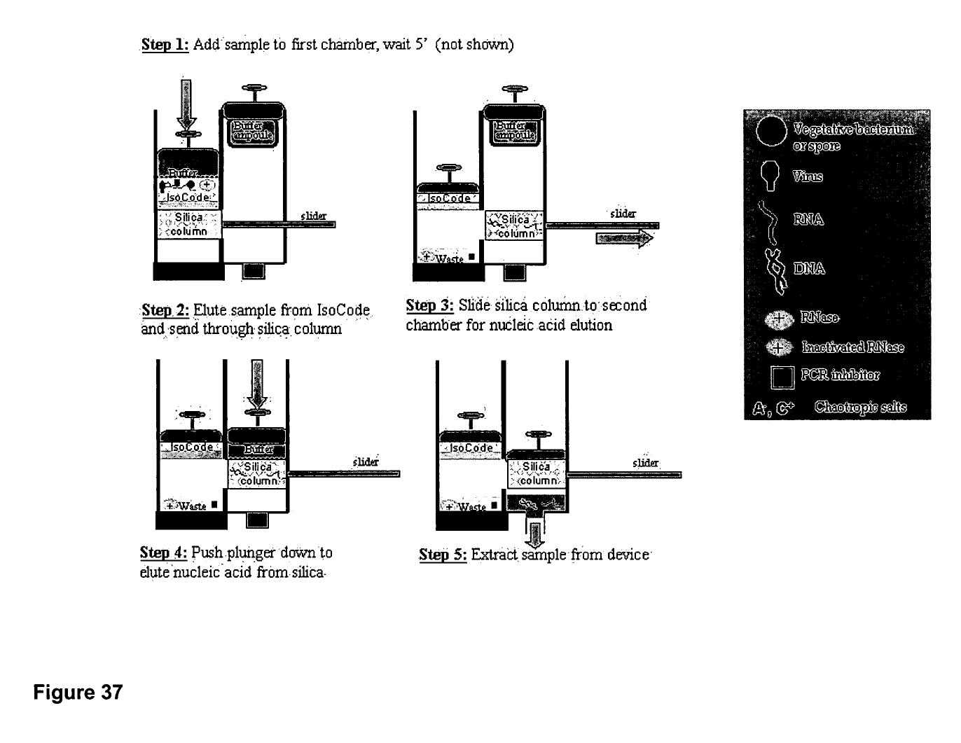

FIG. 37 shows an improved two-chambered (LiNK) device. The improved device contains a silica column to enhance sample purification and concentration.

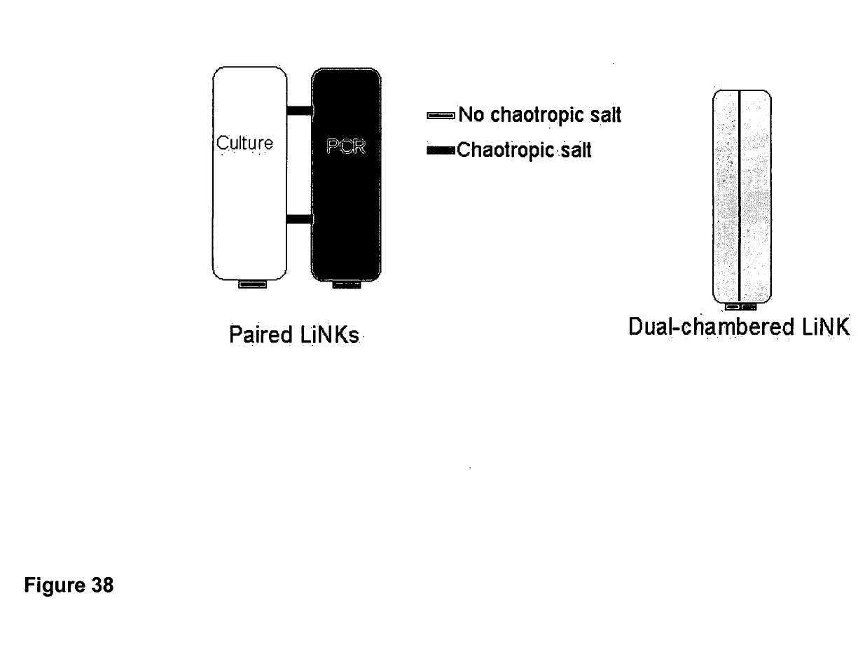

FIG. 38 shows two modified designs for a LiNK-like device. The paired design or the dual-chambered design allow culture of bacterial and other cells within a sample in the absence of chaotropic salts used to facilitate analysis of nucleic acid within the sample.

### DETAILED DESCRIPTION

(i) Overview

The biological, chemical, and environmental sciences often require the analysis of targets which must first be separated or otherwise detected from a heterogeneous population of materials. This process may be further complicated by the presence within a sample of contaminants that may degrade the target or otherwise inhibit the later analysis of the target. The present invention provides methods, compositions, and apparatuses for use in the purification of targets from heterogeneous populations of materials. These methods, compositions, and apparatuses can be used for a wide range of targets (e.g., DNA, RNA, protein, bacteria and bacterial spores (including gram+ and gram−), viruses (including DNA-based and RNA-based), small organic molecules, and chemical compounds) and have a variety of biological, chemical, and environmental applications.

The improved methods and compositions outlined in detail herein greatly enhance the ability to separate or otherwise detect targets from a wide range of gaseous, liquid, and solid samples. Additionally the present invention can be combined with previously described methods and apparatuses that help to maintain the integrity of the target during its separation and prior to further analysis. Such methods and compositions which help maintain the integrity of targets are described in detail in copending US patent publication 2003/0129614, filed Jul. 10, 2003, which is hereby incorporated by reference in its entirety. Briefly, US patent publication 2003/0129614 discloses methods and compositions designed to facilitate isolation and analysis of nucleic acids obtained from samples by processing the samples in the presence of compositions that inhibit agents within samples that can either degrade target or can associate with target and inhibit its further analysis. By way of example, agents within a sample can degrade nucleic acids such as DNA. This degradation both decreases the concentration of DNA in a given sample and also decreases the quality of that DNA such that it may be difficult to process the DNA for further analysis in assays such as PCR.

Applications

There are many potential applications of the methods, compositions, and apparatuses of the present invention. For example, many assays used in forensic sciences require the purification of DNA, protein, or small organic molecules such as non-peptide hormones from amongst a complex sample. Such samples include human or animal fluid or tissues including, but not limited to, blood, saliva, sputum, urine, feces, skin cells, hair follicles, semen, vaginal fluid, bone fragments, bone marrow, brain matter, cerebro-spinal fluid, amniotic fluid, and the like. The purification and further analysis of target from these complex samples is hindered by (a) an often low concentration of target within the sample, (b) degradation of the sample by either environmental contaminants or by agents within the sample which degrade target over time, and (c) the presence of agents within these complex bodily fluids which interfere with techniques needed to analyze the target following its purification. Accordingly, the present invention has substantial application to the forensic sciences and would enhance the ability to analyze biological samples. Additionally we note that the methods and compositions of the present invention can be used effectively to separate target from mixtures of materials that may be present in a “dirty” environment such as soil or water. Accordingly, the present invention facilitates forensic and other studies performed not only on samples of fresh bodily fluids provided directly from individuals or found in a relatively undisturbed environment, but additionally can be used to analyze sample which must be recovered from soil, water (including fresh or salt water), or other sources which may contain a higher concentration of contaminants and other degradatory agents. Accordingly, the methods, compositions, and apparatuses of the present invention are broadly applicable to the analysis of biological materials in a laboratory, hospital, or doctor's office setting, as well to the analysis of biological materials in the field by police, medical examiners, emergency medical technicians, criminal investigators, Haz-mat personnel, and other field-based workers.

The application of the present invention in the biological sciences is not limited, however, to forensics. Advances in medical and genetic testing are already beginning to change the way in which we approach healthcare. A range of diagnostic tests are available or are currently being developed. Such tests rely upon the ability to analyze a particular target (DNA, protein, hormone) contained within a sample of human or animal fluid or tissue. Accordingly, the present invention can be used to further improve the ease and efficiency with which biological samples are analyzed. Additionally, given that the methods and compositions of the present invention allow the separation of smaller quantities of target, use of these methods and compositions in a diagnostic setting will help decrease the amount of sample that must be harvested from a particular patient. Additionally, the present invention provides methods that allow separation of targets from a wide range of samples at previously unattainable speeds and using minimal reagents. The ability to analyze samples quickly and at a reduced cost is advantageous in the health care and medical industry, as well as in many of the other applications of the invention outlined in detail herein.

By way of further example, the present invention can be used to screen blood, blood products, or other pre-packaged medical supplies to insure that these supplies are free from particular contaminants such as bacteria and viruses.

In addition to medical applications, the present invention has a variety of environmental uses. Water, soil, or air samples can be analyzed for the presence of particular targets. Such targets include DNA, RNA, protein, small organic molecules, chemical compounds, bacterial cells or spores (including gram+ or gram−), and viruses (including DNA-based and RNA-based). DNA, RNA, and protein can be derived from humans, non-human animals, plants, bacteria, fungi, protozoa, and viruses. For example, samples of water collected from local ponds, lakes, and beaches can be analyzed to assess the presence and concentration of potentially harmful bacteria or chemical pollutants. Such analysis can be used to monitor the health of these water sources and to evaluate their safety for human recreation. Similarly, samples of soil can be collected and analyzed to assess levels of contamination from natural or industrial sources.

By way of further example, cartridges and filters containing the compositions of the present invention can be used to monitor air and water supplies. Such cartridges and filters can be used to assess air quality in buildings, airplanes, and other closed environments which rely on recirculating air. Furthermore, such cartridges can be used in fish tanks, aquariums, and the like to help monitor water quality and to help pinpoint the source of any changes to water quality.

A final non-limiting example of applications of the present invention can be widely classified in the field of home-land security. Given the threat of warfare employing biological and/or chemical weapons, methods and compositions which can be used to identify the presence of biological or chemical agents in food, water, soil, or air have tremendous possible applications. For example, samples of water and soil surrounding local reservoirs or other likely sources of attack could be collected and analyzed for the presence of biological or chemical contaminants. Furthermore, cartridges and filters can be used to monitor the air (either outside or within buildings, trains, airplanes, or other vehicles) for the presence of biological or chemical contaminants. The invention contemplates that biological contaminants can be identified by either the detection of DNA or RNA from a particular biological agent (such as a bacteria or virus) or by the detection of the bacteria or virus itself. Chemical contaminants may be identified by detection of the organic molecule itself, as well as by detection of its chemical by-products or metabolites. Exemplary biological and chemical agents which may be detected include anthrax, ricin, brucellosis, smallpox, plague, Q-fever, tularemia, botulism, staphylococcus, and viral hemorrhagic fevers including Ebola, mustard gas, Clostridium Perfringens, camelpox, sarin, soman, O-ethyl S-diisopropylaminomethyl methylphosphonothiolate, tabun, and hydrogen cyanide. Exemplary viruses of clinical and environmental relevance can be categorized based on their genome type and whether they are enveloped and include (i) single-stranded, positive sense strand, enveloped, RNA viruses; (ii) single-stranded, positive sense strand, non-enveloped, RNA viruses; (iii) single-stranded, negative sense stranded, enveloped, RNA viruses; (iv) double-stranded, non-enveloped, RNA viruses; and (v) double-stranded, enveloped, DNA viruses. Single-stranded, positive sense strand, enveloped, RNA viruses include, but are not limited to, Eastern equine encephalitis, Western equine encephalitis, Venezuelan equine encephalitis, St. Louis encephalitis, SARS, Hepatitis C, HIV, and West Nile virus. Single-stranded, positive sense stranded, non-enveloped, RNA viruses include, but are not limited to, Norwalk virus, Hepatitis A, and Rhinovirus. Single-stranded, negative sense stranded, enveloped, RNA viruses include, but are not limited to, Ebola, Marburg, and Influenza. Double-stranded, non-enveloped, RNA viruses include, but are not limited to, Rotavirus. Double-stranded, enveloped, DNA viruses include, but are not limited to, Hepatitis B and Variola major.

For each of the potential forensic, medical, diagnostic, environmental, industrial, and, safety applications of the invention outlined above, the invention contemplates the use of the methods, apparatuses, and compositions of the present invention to separate and/or identify target from the heterogeneous sample. Thus, these methods, compositions, and apparatuses are useful not only for further analysis of a particular target and sample, but also for removing a target (e.g., an unwanted target) from a sample. Exemplary uses of the invention for removing target include in decontamination of a sample. Following separation (e.g., removal; physical separation) of all or a portion of a target from a sample, the sample can be handled more safely than prior to removal of the target. The separated target can either be discarded (e.g., discarded appropriately in light of the nature of any hazard that may be associated with the target) or can be further studied using reagents and precautions appropriate in light of the nature of any hazard that may be associated with the target.

(ii) Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “target” is used to refer to a particular molecule of interest. Exemplary targets include DNA, RNA, protein, gram+bacteria, gram−bacteria, bacterial spores, DNA and RNA-based viruses (including retroviruses), small organic molecules (including non-peptide hormones), and chemical compounds. DNA, RNA, and protein can be derived from humans, non-human animals, plants, fungi, protozoa, bacteria, and viruses. For any of the foregoing targets, the invention contemplates the purification of the general class of target (e.g., all DNA in a sample), as well as the purification of a particular species of a class of target (e.g., a particular bacteria or an antibody against a given antigen). In the context of the present invention, the target is that molecule that is substantially purified from a heterogeneous sample using the methods, compositions, and apparatuses of the present invention.

The term “sample” is used to refer to the heterogeneous mixture of biological, chemical, or environmental material. The methods, compositions, and apparatuses of the present invention allow the separation, detection, or substantial purification of a particular target from the sample. A sample can be gaseous, liquid or solid (e.g., either wet solid samples or dry solid sample), and can include biological, chemical, or environmental material. Exemplary biological samples include, but are not limited to, blood, saliva, sputum, urine, feces, skin cells, hair follicles, semen, vaginal fluid, bone fragments, bone marrow, brain matter, cerebro-spinal fluid, and amniotic fluid. Exemplary environmental samples include, but are not limited to, soil, water, non-laboratory-grade environmental water, sludge, air, plant and other vegetative matter, oil, liquid mineral deposits, and solid mineral deposits. The invention further contemplates the application of these methods and compositions in many commercial and industrial applications including the purification of contaminants during food processing or the production of other commercial products.

The term “substrate” is used to refer to any surface which can be modified or otherwise coated with a “surface modifying agent” in order to promote or enhance the interaction between the coated substrate and one or more targets. Substrates may vary widely in size and shape, and the particular substrate may be readily selected by one of skill in the art based on the modifying agent, the target, the sample, and other facts specific to the particular application of the invention. Exemplary substrates include, but are not limited to, magnetic beads, non-magnetic beads, tubes (e.g., polypropylene tubes, polyurethane tubes, etc.), glass slides or coverslips, chips, cassettes, filters, cartridges, and probes including fiber-optic probes.

The surface modifying agent may be coupled to the substrate covalently or non-covalently, and the surface modifying agent may optionally contain a cleavable linker such that the active region of the surface modifying agent can be released from the substrate. The term “active region” is used to refer to the portion of the modifying agent containing a region that interacts with the target. In embodiments in which the modifying agent contains a cleavable linker, cleavage of the linker releases target+the active region of the modifying agent while leaving some portion of the modifying agent attached to the substrate.

The term “Affinity Protocol” or “AP” is used to refer to the method by which a target is substantially purified or otherwise separated from a sample by contacting the sample with a substrate. The surface of the substrate may be coated with a modifying agent to promote or enhance the interaction between the substrate and a specific target.

The term “Affinity Magnet Protocol” or “AMP” is used to refer to embodiments of the AP method in which the substrate has magnetic characteristics. Similarly to substrates used in the AP method, substrates used for the AMP method may be coated with a modifying agent to promote or enhance the interaction between the substrate and a specific target.

The Affinity Protocol and Affinity Magnet Protocol includes a target capture phase where target and substrate interact to form a target-substrate complex. The time required for the binding of target and substrate to form a target-substrate complex is referred to herein as “capture time.” By “binding of target and substrate to form a target-substrate complex” is meant sufficient interaction between target and substrate such that greater than 50% (e.g., at least 51%) of the target in a sample binds to substrate to form a target-substrate complex. In certain embodiments, greater than 60%, 70%, 75%, 80%, 85%, 90%, or greater than 95% of target in a sample binds to substrate to form a target-substrate complex.

In certain applications of the AP and AMP, target-substrate complexes are disrupted and bound target is eluted from the substrate. The time required to elute target from substrate is referred to herein as “elution time.” By “eluting or removing of target from substrate to disrupt a target-substrate complex” is meant disruption of greater than 50% (e.g., at least 51%) of the target-substrate complexes. In certain embodiments, greater than 60%, 70%, 75%, 80%, 85%, 90%, or greater than 95% of target in a sample previously bound to target is eluted.

The term “coupling region” refers to the portion of the modifying agent that interacts with the substrate.

The term “SNAP” or “SNAP method”, or “SNAP protocol” will be used interchangeably throughout to refer to the methods outlined in detail in copending US publication no. 2003/0129614 (U.S. application Ser. No. 10/193,742). As used herein, the use of these terms is not meant to be limited to the use of the particular devices and apparatuses presented in the copending application, but rather is meant to refer to the general method used to isolate a nucleic acid sample under conditions that inhibit degradation of the nucleic acid sample and/or inhibit agents within the sample that interfere with further processing and analysis of the sample (e.g., agents that inhibit analysis of the sample by PCR or RT-PCR).

Herein, the term “aliphatic group” refers to a straight-chain, branched-chain, or cyclic aliphatic hydrocarbon group and includes saturated and unsaturated aliphatic groups, such as an alkyl group, an alkenyl group, and an alkynyl group.

The terms “alkenyl” and “alkynyl” refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “alkylthio” refers to an alkyl group, as defined above, having a sulfur radical attached thereto. Representative alkylthio groups include methylthio, ethylthio, and the like.

The terms “amine” and “amino” are art-recognized and refer to both unsubstituted and substituted amines, e.g., a moiety that can be represented by the general formula: 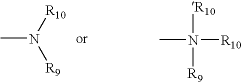
wherein R₉, R₁₀ and R′₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In even more preferred embodiments, R₉ and R₁₀ (and optionally R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The term “amido” is art-recognized as an amino-substituted carbonyl and includes a moiety that can be represented by the general formula: 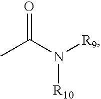
wherein R₉, R₁₀ are as defined above. Preferred embodiments of the amide will not include imides, which may be unstable.

The term “aralkyl”, as used herein, refers to an alkyl group substituted with an aryl group (e.g., an aromatic or heteroaromatic group).

The term “aryl” as used herein includes 5-, 6-, and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The term “carbocycle”, as used herein, refers to an aromatic or non-aromatic ring in which each atom of the ring is carbon.

The term “carbonyl” is art-recognized and includes such moieties as can be represented by the general formula: 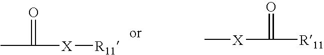
wherein X is a bond or represents an oxygen or a sulfur, and R₁₁ represents a hydrogen, an alkyl, an alkenyl, —(CH₂)ₘ—R₈ or a pharmaceutically acceptable salt, R₁₁ represents a hydrogen, an alkyl, an alkenyl or —(CH₂)ₘ—R₈, where m and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R₁₁′ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The term “heteroatom” as used herein means an atom of any element other than carbon or hydrogen. Preferred heteroatoms are boron, nitrogen, oxygen, phosphorus, sulfur and selenium.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The phrase “protecting group” as used herein means temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

A “selenoalkyl” refers to an alkyl group having a substituted seleno group attached thereto. Exemplary “selenoethers” which may be substituted on the alkyl are selected from one of -Se-alkyl, -Se-alkenyl, -Se-alkynyl, and —Se—(CH₂)ₘ—R₈, m and R₈ being defined above.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g., alkyl, m, n, etc., when it occurs more than once in any structure, is intended to be independent of its definition elsewhere in the same structure.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

“amino acid”—a monomeric unit of a peptide, polypeptide, or protein. There are about eighty amino acids found in naturally occurring peptides, polypeptides and proteins, all of which are L-isomers. The term also includes analogs of the amino acids and D-isomers of the protein amino acids and their analogs.

The term “hydrophobic” refers to the tendency of chemical moieties with nonpolar atoms to interact with each other rather than water or other polar atoms. Materials that are “hydrophobic” are, for the most part, insoluble in water. Natural products with hydrophobic properties include lipids, fatty acids, phospholipids, sphingolipids, acylglycerols, waxes, sterols, steroids, terpenes, prostaglandins, thromboxanes, leukotrienes, isoprenoids, retenoids, biotin, and hydrophobic amino acids such as tryptophan, phenylalanine, isoleucine, leucine, valine, methionine, alanine, proline, and tyrosine. A chemical moiety is also hydrophobic or has hydrophobic properties if its physical properties are determined by the presence of nonpolar atoms.

The term “hydrophilic” refers to chemical moieties with a high affinity for water. Materials that are “hydrophilic” are, for the most part, soluble in water.

As used herein, “protein” is a polymer consisting essentially of any of the about 80 amino acids. Although “polypeptide” is often used in reference to relatively large polypeptides, and “peptide” is often used in reference to small polypeptides, usage of these terms in the art overlaps and is varied.

The terms “peptide(s)”, “protein(s)” and “polypeptide(s)” are used interchangeably herein.

The terms “polynucleotide sequence” and “nucleotide sequence” are also used interchangeably herein.

As used herein, the term “nucleic acid” refers to polynucleotides such as deoxyribonucleic acid (DNA), and, where appropriate, ribonucleic acid (RNA). The term should be understood to include single (sense or antisense) and double-stranded polynucleotides.

The term “small molecule” refers to a compound having a molecular weight less than about 2500 amu, preferably less than about 2000 amu, even more preferably less than about 1500 amu, still more preferably less than about 1000 amu, or most preferably less than about 750 amu.

(iii) Exemplary Methods

The present invention provides an improved method for separating target from a sample so that the target can be further analyzed. This method will be referred to herein as the “Affinity Protocol”, “AP” or the “Affinity Method”. Certain embodiments of this methodology will utilize magnetic substrates and may also be referred to as the “Affinity Magnet Protocol” or “AMP”.

The Affinity Protocol uses substrates to help identify one or more targets from a sample. AP may be used for any of a wide range of targets including, but not limited to, nucleic acids (e.g., DNA and RNA), proteins, bacterial cells or spores (e.g., gram+ and gram−), viruses (e.g., DNA- or RNA-based), small organic molecules (e.g., toxins, hormones, etc), and large chemical compounds. AP may be used to identify target from any of a wide range of samples including gaseous samples (e.g., filtered or unfiltered air), environmental liquid samples (e.g., fresh water, sea water, sludge, mud, re-hydrated soil, gasoline, oil), biological liquid and semi-solid samples (e.g., blood, urine, sputum, saliva, feces, cerebro-spinal fluid, bone marrow, semen, vaginal fluid, brain matter, bone fragments), and environmental solid samples (e.g., dry soil or clay). Additionally, AP may be used to analyze the presence of target on solid surfaces which are not amenable to whole processing. For example, the presence of a target on a desktop, computer keyboard, doorknob, and the like. In such cases, the presence of target can be assessed by first taking a surface wipe of the solid surface, and then processing the surface wipe for the presence of a target. Furthermore, AP may be used to identify target in any of a number of industrial applications such as food processing, chemical processing, or any large scale production effort which would be hindered by the presence of certain contaminating targets within a preparation.

The present invention contemplates that the Affinity Protocol can be used alone to identify target in a sample, and to facilitate the further analysis of that target. For example, the Affinity Protocol can be used to identify the presence of particular bacterial cells in a water sample. These bacterial cells can then be further analyzed cytologically or molecularly.

The Affinity Protocol has many significant advantageous over other methods of isolating or separating targets from heterogeneous samples. Substrates for use in the Affinity Protocol and the Affinity Magnet Protocol are either uncoated (e.g., underivatized) or are derivatized with relatively simple chemical moieties. This is in contrast to many previously available separation techniques which require substrate coated with antibodies immunoreactive with particular targets. Antibodies are more expensive to produce and append to substrates, their use requires tremendous a priori knowledge of the target of interest, and each antibody likely has a narrow spectrum of immunoreactivity. Additionally, the Affinity Protocol and Affinity Magnet Protocol allow rapid separation of target from a heterogeneous sample, and the method requires the use of minimal reagents. These features decrease the cost of the Protocol, and allow its use in the field (e.g., non-laboratory conditions) as well as in the laboratory.

However, the invention further contemplates that the Affinity Protocol can be used in combination with the previously disclosed SNAP method or with other methodologies for further analyzing nucleic acids. The SNAP method, which is outlined in detail in US publication no. 2003/0129614 and is hereby incorporated by reference in its entirety, allows for the isolation of nucleic acids from samples in a manner that prevents their degradation and/or inhibits agents in the sample that interfere with the further analysis of the nucleic acid. An exemplary commercially available product that typifies SNAP-like methodology is IsoCode paper. By coupling the Affinity Protocol with SNAP methodology, the present invention provides a vastly improved method for identifying targets from complex, heterogeneous samples. As the examples provided herein illustrate, the use of both the Affinity Protocol and SNAP methodology, improves the quality of the target identified in a sample and thus facilitates the further analysis of the target. Additionally, the combined methods are more sensitive than the SNAP methodology alone, and thus allow the identification of lower concentrations of target within a sample.

The Affinity Protocol uses substrates that interact with target present in a sample. The substrate may be of virtually any size and shape, and exemplary substrates include beads, tubes, probes, fiber-optics, plates, filters, cartridges, coverslips, chips, films, dishes, swabs, paper or other wipes, and the like. Furthermore, the substrate may be composed of any of a number of materials including, but not limited to, glass, plastic, and silica. The substrate may be magnetized (e.g., possess magnetic characteristics). The substrate may be porous or non-porous, and porous substrates may have any of a range of porosities.

Substrates for use in the Affinity Protocol should have an increased affinity for target in comparison with non-target materials in the sample. As will be detailed herein, some substrates have a higher affinity for certain targets in comparison to certain other targets, and one of skill in the art can readily select a particular substrate depending on factors including the target, the sample, etc. The invention additionally contemplates that the surface of the substrate can be modified to further promote the interaction of the substrate with one or more targets. Moieties that are attached to the surface of a substrate to influence the interaction of the substrate with target are referred to as surface modifying agents. The invention contemplates that one or more surface modifying agents can be appended to the surface of a substrate to promote the interaction of the substrate with a particular target. Exemplary surface modifying agents are provided herein, and in one embodiment of the present invention, a substrate modified with one or more of the surface modifying agents disclosed herein is used in the Affinity Protocol to identify and/or separate a target from a sample.

The invention further contemplates Affinity Protocols which employ a cocktail of substrates. For example, the method may use two or more substrates modified with different surface modifying agents to identify more than one target, and/or the method may use substrates which vary in size, shape, or composition, but are modified with the same surface modifying agent.

To further illustrate the Affinity Protocol, FIG. 1 provides a schematic representation. We note that in the schematized method provided in FIG. 1, a sample is analyzed using both the Affinity Protocol and SNAP methodology to isolate and prepare nucleic acid for further molecular analysis. However, the present invention also contemplates the use of the Affinity Protocol alone to separate any of a number of targets including, but not limited to, DNA, RNA, protein, bacterial cells and spores, viruses, small organic molecules, and large compounds.

In the hypothetical example outlined in FIG. 1, we have a soil sample suspected of containing a particular bacterial target (step 1). The soil sample is taken and combined with water and substrate (step 2). In this example, the substrates are magnetic beads which have an affinity for the suspected bacterial cells. The slurry of soil, water, and beads is mixed to facilitate the interaction between the substrate and the target (step 3). During step 3, target within the sample can associate with the substrate. Following interaction of the target and substrate, target-substrate complexes are separated from the sample. In this example, since the substrates are magnetic beads, the complexes can be readily separated using a magnet (step 4). Steps 1-4 summarize the Affinity Protocol. Following separation of the substrate-target complexes, the target can be analyzed in any of a number of ways depending on the particular target and the type of information that one wishes to obtain. In one embodiment, the Affinity Protocol can be readily combined with SNAP methodology to isolate nucleic acid from the target and process that nucleic acid under conditions that inhibit degradation and/or inhibit agents that prevent further analysis of the nucleic acid. Steps 5-7 demonstrate how SNAP methodology can be combined with the Affinity Protocol.

Identification and/or separation of a target from a sample using a substrate has numerous applications. One of skill in the art will recognize that the term “separation” can have two meanings in the context of the present invention. The term separation can refer to the association of a target with the substrate (e.g., the formation of a target-substrate complex) such that the target is now separated from the remainder of the sample by virtue of its association with the substrate. The term separation can additionally refer to the physical removal of the target and/or target-substrate complex from the remainder of the sample. The invention contemplates embodiments in which either of these are preferred.

The present application provides an improved method (the Affinity Protocol) for identifying and/or separating a target from amongst a heterogeneous liquid, solid, or gaseous sample. As will be appreciated from the examples provided herein, the Affinity Protocol provides an improved method that can be used in a controlled setting such as a laboratory, hospital, or food processing plant, as well as in a less-controlled field setting. The Affinity Protocol is amenable to rapid identification and/or separation, and is amenable to use with any of a large number of substrates which can be chosen based on the specific requirements of the application, sample, and target.

(iv) Exemplary Compositions

As outlined in detail above, in one embodiment of the Affinity Protocol, the surface of the substrate can be modified with a surface modifying agent. Exemplary surface modifying agents can be used to promote the interaction of the coated substrate with target. Preferred surface modifying agents provide an increased affinity between the coated substrate and the target in comparison to either other coated substrates or uncoated substrates.

The invention contemplates that substrates can be coated with any of a number of surface modifying agents, and furthermore that a substrate can be coated with a single surface modifying agent or with more than one surface modifying agents. It is anticipated that some surface modifying agents will have an affinity for a particular class of target (e.g., all DNA or all RNA or all bacterial cells) while other surface modifying agents will have an affinity for a specific target (e.g., a particular bacterial species or the spore versus the cellular form of a particular bacteria or class of bacteria). One of skill in the art can readily test various surface modifying agents and select agents which have the desired affinity for the desired target.

Following the identification of a desired surface modifying agent or agents, any of a number of substrates can be coated or otherwise derivatized such that the surface of the substrate is coated with the surface modifying agent. The invention contemplates that certain surface modifying agents may more readily coat or covalently interact with particular substrates, and thus every surface modifying agent may not be suitable for coating every possible substrate. However, the selection of a suitable substrate for coating with a surface modifying agent can be readily made by one of skill in the art given the particular application, target, sample, etc.

One aspect of the invention is to take a silicon containing surface modifying agent and modify the surface of a substrate to give the surface-modified substrate represented in FIG. 2. The substrate can be modified with any number of surface modifying agents with the degree of surface modification typically expressed as the amount of surface coverage in moles per gram. The substrate can also be modified with more then one type of surface modifying agent by attaching the agents either sequentially or concurrently. The invention contemplates the use of two or more surface modifying agents which both have affinity for the same target, as well as the use of two or more surface modifying agents that have affinity for different targets.

The left panel of FIG. 2 provides a representation of a surface modifying agent, and the right panel provides a representation of a modified substrate. Substrates modified as shown in FIG. 2 can be used to identify and/or separate target (the Affinity Protocol) from any of a range of biological, environmental or chemical sample. For convenience, the representations presented in FIG. 2 use several variables and the invention contemplates the use of surface modifying agents in which these variable are any of the following. We note that for a given structure, the variables are selected as valiance and stability permit.
R1=F, Cl, Br, I, OH, OM, OR, R, NR₂, SiR₃, NCO, CN, O(CO)R
R2=F, Cl, Br, I, OH, OM, OR, R, NR₂, SiR₃, NCO, CN, O(CO)R
R3=F, Cl, Br, I, OH, OM, OR, R, NR₂, SiR₃, NCO, CN, O(CO)R
M=metal
X═NR, O
R=substituted or unsubstituted alkyl, alkenyl, aryl or heteroaryl, hydrogen
Y=a linker/spacer=substituted or unsubstituted alkyl, alkenyl, aryl or heteroaryl, silanyl, siloxanyl, heteroalkyl
Z=F, Cl, Br, I, OH, OM, OR, R, NR₂, SiR₃, NCO, CN, O(CO)R, N(CO)R, PR₂, PR(OR), P(OR)₂, SR, SSR, SO₂R, SO₃R.

The example in FIG. 2 shows the attachment between the silicon containing surface modifying agent and the substrate to occur at only one point. It is well known to those skilled in the art that attachment can occur through the displacement of R₁, R₂, or R₃ including any combination of R₁, R₂, or R₃ to give two or three attachment points between the silicon containing surface modifying agent and the substrate. It is also well known to those skilled in the art that attachment can occur through the displacement of the R₁, R₂, or R₃ of one silicon containing surface modifying agent and a second silicon containing surface modifying agent previously attached to the substrate. Any form of attachment (e.g., covalent or non-covalent) of the silicon containing surface modifying agent to the substrate is acceptable to the practice of this invention.

The surface modifying agent typically contains a coupling region containing a silicon atom bonded to at least one hydrolyzable moiety, optionally a spacer/linker region shown as Y, and an active region shown as Z. The silicon atom is typically substituted with a spacer region shown as Y but this group is optional and Z may be directly attached to the silicon. The silicon is also typically substituted with three groups designated as R₁, R₂, and R₃ which can be identical or different provided that one group is hydrolyzable. Hydrolyzable groups can be, but are not limited to H, F, Cl, Br, I, OH, OM, OR, NR₂, SiR₃, NCO, and OCOR.

The spacer region is typically an alkyl (substituted or unsubstituted), alkenyl, aromatic silane, or siloxane based organic moiety which may be substituted with other organic moieties such as acyl halide, alcohol, aldehyde, alkane, alkene, alkyne, amide, amine, arene, heteroarene, azide, carboxylic acid, disulfide, epoxide, ester, ether, halide, ketone, nitrile, nitro, phenyl, sulfide, sulfone, sulfonic acid, sulfoxide, silane, siloxane or thiol. The alkyl, alkenyl, or aromatic based organic moiety may contain up to 50 carbon atoms and contains more preferably up to 20 carbon atoms and contains most preferably up to 10 carbon atoms. The silane or siloxane based silicon moiety may contain up to 50 silicon or carbon atoms and contains more preferably up to 20 silicon or carbon atoms and contains most preferably up to 10 silicon or carbon atoms. Attached to the Y spacer region, or optionally directly to the silicon, is the active region shown as Z. The active region is employed to attract and bind the organism or biological molecule of interest (the target). The binding of target to the active region can occur via any of a number of interactions. Without being bound by theory, the binding between the active region and target can occur via van der Waals interactions, hydrogen bonding, covalent bonding, and/or ionic bonding.

Additionally, we note that the active region can also contain an alkyl, alkenyl, or aromatic based organic moiety which may be substituted with other organic moieties such as acyl halide, alcohol, aldehyde, alkane, alkene, alkyne, amide, amine, arene, heteroarene, azide, carboxylic acid, disulfide, epoxide, ester, ether, halide, ketone, nitrile, nitro, phenyl, sulfide, sulfone, sulfonic acid, sulfoxide, silane, siloxane or thiol. The alkyl, vinyl, or aromatic based organic moiety may contain up to 50 carbon atoms and contains more preferably up to 20 carbon atoms and contains most preferably up to 10 carbon atoms.

A second aspect of the invention is to take a silicon containing surface modifying agent and modify the surface of a substrate to give the material shown in FIG. 3. In this aspect of the invention the number of active regions in the surface modifying agent is more than one with each separated by a spacer region. It is recognized that when more than one active region is employed on the surface modifying agent, the active regions cans be attached in either a linear manner or in a branched manner from the spacer/linker region. The invention further contemplates that more than one active region can be attached to a spacer region and that the spacer region can itself be branched. The number of active regions on a surface modifying agent can be any number from 2 to 1000 with a preferred range from 2 to 100, a more preferred range from 2 to 20 and a most preferred range from 2 to 5.

The active regions on the surface modifying agent can be the same or different and the spacer regions on the surface modifying agent can be the same or different. The substrate can be modified with any number of surface modifying agents with the degree of surface modification typically expressed as the amount of surface coverage in moles per gram. The substrate can also be modified with more then one type of surface modifying agent by attaching the agents either sequentially or concurrently.

The left panel of FIG. 3 provides a representation of a surface modifying agent, and the right panel provides a representation of a modified substrate. Substrates modified as shown in FIG. 3 can be used to identify and/or separate target (the Affinity Protocol) from any of a range of biological, environmental or chemical sample. For convenience, the representations presented in FIG. 3 use several variables and the invention contemplates the use of surface modifying agents in which these variable are any of the following. We note that for a given structure, the variables are selected as valiance and stability permit.
R1=F, Cl, Br, I, OH, OM, OR, R, NR₂, SiR₃, NCO, CN, O(CO)R
R2=F, Cl, Br, I, OH, OM, OR, R, NR₂, SiR₃, NCO, CN, O(CO)R
R3=F, Cl, Br, I, OH, OM, OR, R, NR₂, SiR₃, NCO, CN, O(CO)R
M=metal
X═NR, O
R=substituted or unsubstituted alkyl, alkenyl, aryl or heteroaryl, hydrogen
Y=substituted or unsubstituted alkyl, alkenyl, aryl or heteroaryl, silanyl, siloxanyl, heteroalkyl
Z=F, Cl, Br, I, OH, OM, OR, R, NR₂, SiR₃, NCO, CN, O(CO)R, N(CO)R, PR₂, PR(OR), P(OR)₂, SR, SSR, SO₂R, SO₃R.

For substrates modified with either the modifying agents represented in FIG. 2, the modifying agents represented in FIG. 3, or other modifying agents, the invention contemplates that any substrate can be modified. Additionally, the size and shape of the substrate can be altered and selected based on the particular application of the technology. Exemplary shapes include spherical, irregular, and rod shaped, and the size and shape refer to that of the average substrate. The substrate can be either solid, pitted, or porous, and one of skill in the art will readily recognize that this will influence the substrate surface area and will thus affect the amount of surface coverage possible. It is understood that the substrate size will vary about the average and that in some aspects of this invention a mixture of substrate sizes may be advantageous. For example, in some embodiments, the use of coated beads of various sizes may be advantageous. In general the substrate size can range from 0.01 to 100 mm. In some applications, the substrate diameter will range from 0.5 to 10 mm, from 1 to 5 mm, or from 1 to 2 mm. In other applications, the substrate diameter will be preferred to range from 0.01 to 500 μm, from 0.1 to 120 μm, or from 1 to 50 μm. However, the invention additionally contemplates the modification of larger surfaces such as plates and dishes, as well as the adaptation of the methods and compositions of the invention for large-scale industrial applications.

The substrate can be made of any material. Preferred substrates have a surface composed in whole or in part of a metal oxide, a hydroxide, or a halide. Those skilled in the art will recognize that any metal oxide surface can contain hydroxide functionality either innately or through a treatment to partially hydrolyze the metal oxide. Furthermore, any metal halide can also contain hydroxide functionality either innately or through a treatment to partially hydrolyze the metal halide. Organic surfaces can also be employed in this invention provided the surface has a hydroxide moiety either present or in latent form. A preferred material is a material that contains silicon oxides or silicon hydroxide either with or without the presence of other metals or metal oxides or metal halide. Additional substrates for use in the methods of the present invention include glass and plastic.

In some aspects of the invention, the substrate will contain material in sufficient quantity to make the substrate paramagnetic (herein referred to as possessing magnetic character) in that the substrate is attracted to magnetic fields. In a preferred form of the invention, the substrate will contain iron, nickel, or cobalt, and in a more preferred form the substrate will contain iron or an iron oxide. In this aspect the use of a paramagnetic substrate is advantageous in that a magnetic field can be used to separate the magnetic substrate from other non-magnetic materials.

In some other aspects of the invention the substrate will contain a perforation such that a string that can be passed through the substrate. Such a string, tether or other linking means can connect substrates together and can be used to facilitate later recover of either the substrate or of the substrate-target complexes.

There are aspects of this invention in which it would be advantageous to detach the active region of the surface modifying agent from the substrate. Accordingly, the invention contemplates modifying agents that contain a cleavable linker. The presence of a cleavable linker allows the release of the active region of the modifying agent+target from the remainder of the substrate. The ability to release the target in this way may greatly facilitate the further analysis of the target. For example, the ability to release the target may be especially important in scenarios in which the association between the substrate and the target is very strong.

The method of detachment can include treatment of the surface modified substrate with any process or chemical that disrupts or reverses the binding forces that attract the target and the active region. These include altering the pH or salt concentration, exposing the complex to heat, and exposing the complex to light. We note that the use of such methods does not disrupt or cleave the modifying agent itself, but rather releases the target from the active agent while leaving the modifying agent intact.

In other aspects, the invention contemplates that the release of target involves cleavage within a site in the modifying agent (e.g., cleavage of the linker and release of the active region+target). This can be accomplished by cleaving a covalent bond in the spacer region thereby separating the active region of the surface modifying agent from the substrate. This may also be accomplished by cleaving covalent bonds in the coupling region thereby separating the active region of the surface modifying agent from the substrate. Particular specific examples of methods that can be used to induce a cleavage event within the modifying agent can be found in the Examples.

(v) Exemplary Screening Assays

The invention provides an Affinity Protocol for identifying and/or separating target from a sample. The substrate can be modified in any of a variety of ways to further promote the interaction of the substrate with a particular target. For example, the surface of the substrate can be modified with one or more surface modifying agents such as the amine-containing agents provided herein.

Given the identification of a number of surface modifying agents that promote interaction of a target with the modified substrate, the present invention contemplates screens to identify further agents that can be used as modifying agents. Armed with an appropriate assay or assays to allow the relatively efficient evaluation of substrate coatings, one of skill in the art can readily screen any of a number of coatings and identify coatings that may be useful for promoting the interaction of substrate with a particular target. For example, one could specifically screen for coatings that promote the interaction of substrate with DNA, RNA, bacterial cells and spores generally, or a particular bacterial cell or spore.

We provide several screening assays that can be used to efficiently identify surface modifying agents for use in the Affinity Protocol. Substrates modified with candidate surface modifying agents can be screened using any of these assays, and the ability of substrates coated with one or more of the candidate surface modifying agents to interact with a target can be assessed. Substrates coated with candidate agents that interact with a particular target with a greater affinity than that of the uncoated substrate may be further analyzed to determine their target specificity, ease of manufacture, etc.

Assay 1—Flow Cytometry Screening Assay. The following protocol, represented schematically in FIG. 4, is representative of an assay that can be used to readily assess the usefulness of a number of candidate substrate coatings. Bacteria are cultured in appropriate conditions to late log or stationary phase and fluorescently stained. A sample of the bacteria (10⁵ to 10⁷ cells per ml give standard deviations less than 15%) are counted using the flow cytometer to give an initial concentration. The bacteria are mixed with coated substrate in a volume of phosphate-buffered saline (PBS) at varying pH (2, 7, 10) or deionized water (pH 5). Depending on the substrate coating, some amount of the bacteria will adhere to the beads. Following mixing of the substrate and target, the samples are filtered slowly through a 5 μm PVDF syringe filter (Millipore) to remove substrate with bound cells and allow free cells to pass through the filter into a tube. Filter size may be adjusted based on target size and bead size for efficient separation. The unbound bacteria that pass through the filter are analyzed by flow cytometry, and the percent of bacteria removed by the beads is calculated (FIG. 4). A sample of the bacteria are also passed through the same type of filter without the addition of substrate as a control.

Using this type of assay, a large number of substrate coatings can be rapidly assessed and compared. Candidate coatings worth further analysis are those that bind bacterial cells more readily (e.g., promote the interaction between target and substrate) than uncoated substrate.

Counting bacteria by flow cytometry was found to be reproducible between samples, and cell densities calculated by flow cytometry agreed with expected cell densities as determined by light microscopy within two standard deviations.

Assay 2—Fluorescence Screening Assay. The following protocol, represented schematically in FIG. 5, is representative of a second assay that can be used to readily assess the usefulness of a number of candidate substrate coatings. In order to quantify the affinity of substrates towards nucleic acids, a fluorescence technique was developed that can be used to quantify the percentage of dsDNA captured by a particular coated substrate. An important application of this assay is in evaluating currently available and novel coatings for their utility as surface modifying agents.

Place a suitable volume of an appropriate mixing buffer in a centrifuge tube. The buffer can be selected based on the particular sample and target. Measure the amount of dsDNA prior to the addition of any substrate. For an in vitro screening assay, a starting concentration of dsDNA in the range of 50 pg/ml-1 μg/ml is appropriate. Add Pico-green dsDNA intercalating dye to the dsDNA. Pico-green has an excitation wavelength of 488 nm and an emission wavelength of 522 nm. Other fluorescent intercalating dyes can also be used and one of skill in the art can select a dye that has appropriate excitation and emission characteristics for easy laboratory analysis. Other commonly used, fluorescent intercalating dyes include, but are not limited to, Acridine Orange, Propidium Iodine, DAPI, SYBR Green 1, and ethidium bromide. Following addition of dye, allow dye and DNA to mix, and measure the fluorescence. This provides a baseline for the analysis.

Add coated substrate to the labeled DNA sample and allow substrate and sample to mix. Shake and vortex for approximately 30 seconds to allow adhesion to occur. Separate substrate from free DNA by centrifugation or settling, and measure the fluorescence of DNA remaining in solution.

By comparing the fluorescence of the DNA mixture before and after the addition of the coated substrate, one can quantify the capture efficiency of each coated substrate. This allows the evaluation of any of a number of substrate coatings.

Assay 3—PCR Screening Assay. PCR can also be used to determine adhesion by determining the cycle number of a sample before and after the addition of coated substrate. The steps are similar to those outlined above for the fluorescence assay, except staining of the DNA with an intercalating agent is not required. A sample of the initial stock solution of DNA and a sample of the supernatant removed following substrate addition and mixing are compared by PCR. An increase in the cycle number required to amplify DNA from a sample following addition of substrate indicates that DNA adhered to the substrate.

(vi) Exemplary Apparatuses

The present invention provides two classes of apparatuses. The first class of devices is designed to facilitate the efficient interaction of modified substrate with large amounts of sample. Such devices are useful for applications of the Affinity Protocol in large-scale industrial settings in which it may be difficult to readily contact a substrate with a sample containing a particular target, and is especially important when the target may not be evenly distributed throughout the entire sample.

The Affinity Protocol and Affinity Magnet Protocol described in detail herein use substrates such as beads to capture target from materials such as liquids, slurries, and air. Large quantities of sample material require effective mixing to maximize substrate-target interaction and capture efficiency on the bead surfaces. The first class of device of the present invention was designed based on modifications of known techniques for mixing viscous slurries. These techniques use the principle of chaotic mixing, and are known as journal bearing flow (which refers to the flow of fluids in a journal bearing—a hollow cylinder enclosing a solid shaft that rotates about its axis). Journal bearing flow is typically used to mix viscous fluids such as oils and cement, in large (multi-gallon) quantities. The principle is to place the material in a cylindrical container with an annulus, formed by placing a second cylinder inside the first. The two cylinders are aligned eccentric to each other, and are co- or counter-rotated about their longitudinal axes at slow speeds (typically less than 20 revolutions per minute). The slow rotation causes the material inside the annulus to stretch and fold, thereby decreasing the interaction distance between any two particles in the material. Over the course of many rotations, efficient mixing can be achieved. FIG. 6 illustrates the configuration of the cylinders, and shows the results of a simulation which demonstrates the fairly uniform particle distribution following mixing.

FIG. 7 schematically illustrates the application of this principle to a particular scenario where a target within a soil sample is being analyzed. The sample and substrate are mixed with water to form a slurry. The substrate is mixed throughout the sample using chaotic mixing methods. The substrate is then extracted from the sample, and released into water or other buffer.

A particular apparatus designed to facilitate mixing of substrate and sample is described in detail in the examples section of this application. Furthermore, the examples provide data demonstrating the performance of this device in a representative scenario. The invention contemplates multiple variations on this class of devices which are referred to herein as “Class I apparatuses”, “Class I devices”, “Chaotic Mixing apparatus”, or “Chaotic Mixing device”. The device can be of virtually any size, and the size of the device can be scaled up or down depending on the total volume of sample which must be accommodated. The key aspect of the device is not its overall size, but rather (a) the presence of two eccentrically placed cylinders, (b) an outer cylinder which is larger than an inner cylinder, and (c) the rotation of the cylinders at relatively low speeds. The cylinders may vary in size and shape, and the two cylinders need not have the same shape. Additionally, one or both cylinders can be altered to increase its surface area by, for example, the addition of fins, vanes, or ribs to the outer surface of the inner cylinder and/or to the inner surface of the outer cylinder. Such fins or vanes not only increase the surface area but can also increase vertical circulation of the sample during mixing, thereby increasing substrate-target interaction.

The invention contemplates that the cylinders can be either solid or hollow, and whether the cylinder should be solid or hollow can be determined based on the size of the cylinders and based on the material used to construct the cylinder. These factors will influence the weight and strength of the cylinders, as well as the cost of their construction. The cylinders can be constructed from any of a number of materials, and the two cylinders need not be constructed of the same materials. The materials can be selected based on the size and shape of the cylinders, as well as the particular type of sample, substrate and target. Exemplary materials include, but are not limited to, Teflon, stainless steel, iron or other metal, and plastic. Additionally, the invention contemplates that the cylinders can be plated with a material such as gold, platinum, iron, Teflon, and the like, to improve particular characteristics of the cylinders.

The rotation of the cylinders can be in the same direction or in opposite directions (e.g., both cylinders can be rotated clockwise, both cylinders can be rotated counter-clockwise, or one cylinder can be rotated clockwise while the other is rotated counter-clockwise). The rotation of the cylinders should occur at relatively slow speeds ranging from 5-50 rpm, preferably from 10-20 rpm. The rotation of the cylinders in exemplary devices should occur at 10, 11, 12, 13, 14, 15, 16, 17, 18, 19, or 20 rpm, however, the invention contemplates that the optimal rotation can be selected based on the particular sample, the total volume being mixed, and the particular target.

The invention further contemplates that the dynamics of the beads as they are circulated through the mixture can be influenced by using a varying external magnetic field, such as a rotating magnetic field external to the outer cylinder. This may be especially useful when the substrate has a magnetic character (e.g., coated or uncoated magnetic beads). In a further application of the use of magnetic fields in these devices, the inner cylinder can serve a dual purpose by being constructed as an electromagnet, with a coil of wire wrapped around an iron-based core. When the electromagnet is activated, the inner cylinder can serve as a collection rod for the substrate in embodiments which use a substrate with a magnetic character. In this way, the inner cylinder can serve two functions as both an instrument to facilitate mixing of substrate and target and as a means for collecting substrate-target complexes following mixing.

The invention further contemplates a second class of devices. These devices comprise filters or cartridges that contain one or more substrates. The design of filters and cartridges containing one or more substrates capable of interacting with targets will facilitate the monitoring and analysis of a variety of air and liquid samples. For example, such filters and cartridges will allow a more detailed analysis of air that circulates in buildings, airplanes, and public transportation vehicles, as well as the analysis of water in reservoirs and streams.

The invention contemplates that Affinity Protocol-adapted filters and cartridges can be used alone, in combination with previously disclosed filters and cartridges that facilitate the analysis of DNA (see, US publication no. 2003/0129614, hereby incorporated by reference in its entirety), and in combination with other commercially available filters used to analyze air and water (e.g., HVAC air filters, HEPA filters, charcoal-based water filter, and the like).

FIGS. 27-30 provide drawings of some exemplary filter and cartridge designs. However, the present invention contemplates a range of filter and cartridge designs. In some embodiments, the cartridge or filter contains multiple layers of substrates. Each layer may contain either the same substrate, or different substrates. In other embodiments, the cartridge or filter contains only a single layer, however, that single layer may optionally containing multiple substrates or a single substrate modified with multiple surface modifying agents.

Of particular note, as with all of the substrates and modified substrates of the present invention, the Affinity Protocol adapted filters and cartridges are amenable to use under a range of conditions, can be readily changed or processed for analysis, and can be used at the bench (e.g., in a doctor's office, hospital, laboratory, processing plant) or in the field (e.g., at a site of suspected contamination, on the runway of an airport, at a crime scene).

### EXEMPLIFICATION

The invention now being generally described, it will be more readily understood by reference to the following examples which are included merely for purposes of illustration of certain aspects and embodiments of the present invention, and are not intended to limit the invention.

### Example 1

### Application of the Affinity Protocol

As outlined in detail above, the Affinity Protocol provides an improved method for identifying targets in a sample. The protocol can be used either alone or in combination with SNAP methodology, can be used to identify a wide range of targets from a diverse array of samples, and can be used with a variety of substrates. One substrate that can be useful for identifying particular targets is commercially available magnetic beads. Such beads are available from a number of manufacturers, come in a range of sizes and shapes, and are composed of any of a number of materials. Each of these factors can be optimized based upon the particular target, sample, and other factors.

The following methodologies briefly summarize methods employed to use commercially available magnetic beads as a substrate in the Affinity Protocol. Commercially available magnetic beads are shipped in a buffer. Prior to use, the beads were washed as follows: place 1 mL magnetic beads in a microcentrifuge tube, pellet beads at maximum (14,000 rpm) microcentrifuge speed, remove all liquid from above the bead pellet, resuspend in distilled water, and repeat as necessary to wash beads.

To perform the Affinity protocol on liquid samples as outlined schematically in FIG. 1, one must obtain a liquid sample containing a particular target of interest. Vortex the sample briefly to mix, and place a portion of the sample into a microfuge tube. For solid samples such as soil samples, obtain the sample and place into microfuge tube. Add filtered distilled water to the sample and mix to create a slurry.

Following initial preparation of sample, add prepared magnetic beads to the tube containing the sample and close the tube. Place the tube with sample and beads in a rotating mixer for 10-20 minutes. Use the collection magnet to draw the beads to the side of the tube, taking enough time to ensure all beads have migrated. Collection time should be 10-20 seconds. Using a pipettor with a filter tip, remove all but a small volume of liquid from the tube, taking care not to disturb the pellet of magnetic beads collected at the side of the tube. Gently resuspend the substrate (which should be bound to target) using the small volume of liquid left behind in the previous step. After the target-substrate complex is resuspended, remove all of the liquid (containing target-substrate complex) and apply to commercially available medium such as Isocode paper (this allows the performance of SNAP methodology on your sample).

Following the Affinity Protocol steps outlined in detail above, nucleic acid from the sample can be processed using the Isocode paper or other SNAP methodology, and then the nucleic acids can be analyzed via PCR or other commonly employed technique for analyzing nucleic acids. Briefly, dry the Isocode paper triangles in dishes, using one of four methods: place dishes (uncovered) with triangles in a vacuum oven at 60°±5° C. for 15 minutes, place dishes (uncovered) with triangles in an incubator at 60°±5° C. for 15 minutes (ensure that there is no water in the humidity tray), place dishes (uncovered) with triangles in a biosafety hood at room temperature until completely dry, or place each dish with triangle in a sealed pouch with a desiccant packet at room temperature until completely dry. After the sample has been dried, continue processing with SNAP protocol for elution of target from IsoCode and analyze nucleic acid by PCR or other commonly used molecular biological approach.

### Example 2

### Preliminary Analysis of Surface Modifying Agents—Analysis of Commercially Available Substrates

We conducted an initial screening of 19 commercially available magnetic beads of varied coatings and sizes (Table 1) to ascertain their usefulness in the Affinity Protocol. The goal was to determine which commercially available beads provided the best overall efficiency in increasing signal (decreasing cycle number using PCR) in comparison to that achieved by the use of the SNAP protocol alone. The identification of the characteristics of commercially available substrates and coatings that provide increased efficiency in the separation and identification of nucleic acid from various samples can be used to develop a rationale strategy for designing additional substrates and coatings. In these experiments using commercially available beads, the efficacy of each bead was assessed in comparison to the analysis of target with SNAP alone. Binding efficiency of each bead was evaluated using the fluorescence and flow cytometry assays described above.

TABLE 1
Commercially Available Magnetic Beads

| Bead # | Company | Description | Size (μm) |
|---|---|---|---|
| 1 | Cortex Biochem | PS-DVB-Amine-Amide | 3.2 |
| 2 | Cortex Biochem | PS-DVB-COOH-Aryl acid | 3.2 |
| 3 | Cortex Biochem | Polyacrylamide on Charcoal | 1-25 |
| 4 | Cortex Biochem | Cellulose | 1-10 |
| 5 | Cortex Biochem | Acrolein | 1-10 |
| 7 | AB Gene | Polystyrene-COOH | 3.5 |
| 8 | MPG | Silica | 0.5-5.0 |
| 9 | BioSource | Streptavidin | 1 |
| 10 | Bugs n′ Beads | Polyvinylalcohol | ˜1 |
| 11 | Dynabeads | PS-Amine | 2.8 |
| 12 | Polysciences | COOH | ˜1 |
| 13 | Polysciences | COOH | ˜1 |
| 14 | Sperotech | PS-COOH (smooth/encap/ | 3.0-3.2 |
| | | no xlink) | |
| 15 | Sperotech | PS-COOH (encap/no xlink) | 3.0-3.2 |
| 16 | Sperotech | PS-COOH (encap/no xlink) | 1.5-1.9 |
| 17 | Sperotech | PS-COOH (encap/no xlink) | 1.1-1.4 |
| 18 | Sperotech | PS-COOH (encap/no xlink) | 4.0-4.5 |
| 19 | Sperotech | PS (encap/no xlink) | 4.0-4.5 |
| | Cortex Biochem | Carboxymethyl cellulose | 1-10 |
| | | (Cat. exch.) | |
| | Cortex Biochem | Diethylaminoethyl cellulose | 1-10 |
| | | (An. Exch.) | |
| | Cortex Biochem | Amine precursor to Streptavidin | ˜1 |

Briefly, FIG. 8 summarizes the results of analysis of commercially available magnetic beads. The data was normalized to the signal for samples analyzed by SNAP alone so that the graphical representation presented in the figure demonstrates which beads enhanced signal versus SNAP alone. Soil samples were seeded with 10⁴ cells/g soil of vegetative B. anthracis. The soil samples were contacted with the beads which bound the bacterial cells with varying affinities.

FIG. 8 demonstrated that the combination of Affinity Protocol and SNAP technology enhances the analysis of samples in comparison to SNAP alone. Furthermore, the figure demonstrates that certain surface modifying agents are capable of further enhancing the interaction between substrate and target.

We also examined several commercially available non-magnetic beads. We note that although a large number of beads were initially screened, only those of 50 μm size were directly compared and data reported.

TABLE 2
Commercially Available Non-Magnetic Beads

| Bead # | Company | Description | Size (μm) |
|---|---|---|---|
| 1 | Aldrich | aminopropyl silica (NH2) | 50 |
| 2 | Aldrich | chloropropyl silica (Cl) | 50 |
| | Aldrich | celite | n/a |
| 3 | YMC | diol (OH2) | 50 |
| 4 | YMC | silica (YMC) (SiO) | 50 |
| | Aldrich | amberlyst 36 Strong Anion Exchange | >1 μm |
| | Aldrich | amberlite ICR Cation Exchange | >1 μm |
| | Aldrich | amberlite IRC Anion Exchange | >1 μm |
| | Aldrich | alumina neutral | 25-50 |
| | Aldrich | alumina slightly acidic | 25-50 |
| | Aldrich | alumina acidic | 25-50 |
| | Aldrich | alumina basic | 25-50 |
| 5 | YMC | amine (NH2) | 50 |
| | YMC | amine (NH2) | 10 |
| 6 | CPG | aminopropyl (NH2) | 40-70 |
| 7 | CPG | long chain amine (15A) (NH2) | 40-70 |
| 8 | CPG | glyceryl (OH2) | 40-70 |
| 9 | CPG | carboxyl (COOH) | 40-70 |
| 10 | CPG | carboxymethyl (COOMe) | 70-120 |
| 11 | CPG | silica (SiO) | 40-70 |

The efficacy of these beads was assessed by measuring the percentage of DNA that adhered to the bead following incubation of the bead with a sample, and these results are summarized in FIG. 9. We note that amine-functionalized beads augmented the interaction between substrate and DNA. Accordingly, and as detailed herein, the present invention designed a variety of other amine-functionalized surface modifying agents, and contemplates that other amine-functionalized surface modifying agents can also be designed to promote the interaction between substrate and target—particularly between substrate and nucleic acid.

We note that although the interaction of substrate with DNA was directly tested in this experiment, the interaction of substrate with other nucleic acids such as RNA can also be evaluated. Based on the chemical structure of RNA, substrates that interact with DNA are likely to interact with RNA, and may be used to separate target RNA from a sample. Methodologies in which RNA is the target may be further modified to prevent the degradation of RNA which is generally less stable than DNA.

### Example 3

### Preparation of Amine-Containing Surface Modifying Agents

Following our analysis of commercially available beads (e.g., substrates) containing various commercially available coatings, we prepared a variety of novel coated substrates to assess the usefulness of these coated substrates in the Affinity Protocol. Specifically, we focused on amine containing surface modifying agents, however, similar experiments can be readily performed using other classes of surface modifying agents. As detailed herein, we prepared a number of surface modifying agents and used these agents to modify substrates of various sizes, shapes, and materials.

A. Preparation of 50-Micrometer Surface Modified Silica Gel

A slurry was prepared from 2.0 grams of 50-μm particle size silica gel purchased from Waters Corporation (YMC-gel silica) and 20 ml of isopropyl alcohol. To the slurry was added 10 mmole of the surface modifying agent. The slurry was gently stirred for 16 hours and then filtered. The silica gel was resuspended in 20 ml of isopropyl alcohol and filtered two additional times to remove unreacted surface modifying agent. The surface modified silica gel was dried overnight in a vacuum oven at 50° C. The amount of surface modification was determined by thermogravimetric analysis. Table 3 lists the surface modifying agents employed and the resulting surface coverage determined for modified 50-μm particle size silica gel. The W designation indicates that the resultant substrate is modified Waters Corporation silica gel, and the letters are used to indicate the surface modifying agent employed.

TABLE 3

| | | Surface Coverage |
|---|---|---|
| Sample | Surface Modifying Agent | (mmole/gm) |
| W-A | 3-aminopropyltrimethoxysilane | 1.00 |
| W-B | (3-trimethoxysilylpropyl)diethylenetriamine | 0.63 |
| W-C | N-(2-aminoethyl)-3-aminopropyltrimethoxysilane | 0.76 |
| W-D | N-trimethoxysilylpropyl-N,N,N-trimethylammonium chloride | 0.61 |
| W-E | bis(2-hydroxyethyl)-3-aminopropyltriethoxysilane | 0.45 |
| W-F | (N,N-dimethylaminopropyl)trimethoxysilane | 0.79 |
| W-G | N-(3-triethoxysilanepropyl)-4,5-dihydroimidazole | 0.50 |
| W-H | 2-(trimethoxysilylethyl)pyridine | 0.46 |
| W-I | (aminoethylaminomethyl)phenethyltrimethoxysilane | 0.75 |
| W-J | 2-(diphenylphospino)ethyltriethoxysilane | 0.29 |
| W-K | tetradecyldimethyl(3-trimethoxysilylpropyl)ammonium chloride | 0.30 |
| W-L | diethylphosphatoethyltriethoxysilane | 0.33 |
| W-M | 3-mercaptopropyltrimethoxysilane | 0.47 |
| W-N | N-phenylaminopropyltrimethoxysilane | 0.09 |
| W-O | N-(6-aminohexyl)aminopropyltrimethoxysilanetrimethoxysilane | 0.66 |
| W-R | N-(trimethoxysilylpropyl)ethylenediamine, triacetic acid, trisodium | 0.15 |
| | salt | |
| W-S | N-(2-aminoethyl)-11-aminoundecyltrimethoxysilane | 0.67 |
| W-T | N-(3-triethoxysilanepropyl)gluconamide | 0.66 |
| W-U | N-(triethoxysilanepropyl)-O-polyethylene oxide urethane | 0.15 |
| W-V | 3-(trihydroxysilyl)-1-propanesulfonic acid | 0.09 |
| W-W | carboxyethylsilanetriol | 0.24 |
| W-X | N,N-didecyl-N-methyl-N-(3-trimethoxysilylpropyl)ammonium | 0.37 |
| | chloride | |
| W-Y | 2-[methoxy(polyethyleneoxy)propyl]trimethoxysilane | 0.15 |

B. Preparation of 1-Millimeter Surface Modified Soda Lime Glass Beads A suspension was prepared from 2.0 grams of 1-mm soda lime glass beads from PGC Scientific and 2 ml of 10% aqueous nitric acid and allowed to reflux with gentle stirring for 30 minutes. The nitric acid solution was decanted off and the beads were filtered and washed with deionized water. The beads were then added to 2 ml of 10 N sodium hydroxide and allowed to reflux with gentle stirring for 120 minutes. The sodium hydroxide solution was decanted off and the beads were filtered and extensively washed with deionized water. The beads were dried under vacuum for 4 hours at 100° C.

A suspension was prepared from the dried beads, 1 ml of the surface modifying agent, and 19 ml of dry toluene. The suspension was gently stirred for 45 minutes and filtered. The beads were washed with toluene, washed with ethanol, and vacuum dried for 3 hours at room temperature and 30 minutes at 100° C. The amount of surface modification was determined by performing a Kaiser test and following the change in absorbance at 575-nm. Table 4 lists the surface modifying agents employed and the resulting surface coverage determined for modified 1-mm soda lime glass beads. The PS designation indicates that the resultant substrate is modified PGC soda lime glass beads, and the letters are used to indicate the surface modifying agent employed.

TABLE 4

| | | Surface Coverage |
|---|---|---|
| Sample | Surface Modifying Agent | (μmole/gm) |
| PS-B | (3-trimethoxysilylpropyl)diethylenetriamine | 0.52 |

C. Preparation of 1-Millimeter Surface Modified Borosilicate Glass Beads

A suspension was prepared from 2.0 grams of 1-mm borosilicate glass beads from PGC Scientific and 2 ml of 10% aqueous nitric acid and allowed to reflux with gentle stirring for 30 minutes. The nitric acid solution was decanted off and the beads were filtered and washed with deionized water. The beads were then added to 2 ml of 10 N sodium hydroxide and allowed to reflux with gentle stirring for 120 minutes. The sodium hydroxide solution was decanted off and the beads were filtered and extensively washed with deionized water. The beads were dried under vacuum for 4 hours at 100° C.

A suspension was prepared from the dried beads, 1 ml of the surface modifying agent, and 19 ml of dry toluene. The suspension was gently stirred for 5 hours and filtered. The beads were washed with toluene, washed with ethanol, and vacuum dried for 3 hours at room temperature and 30 minutes at 100° C. The amount of surface modification was determined by performing a Kaiser test and following the change in absorbance at 575-nm. Table 5 lists the surface modifying agents employed and the resulting surface coverage determined for modified 1-mm borosilicate glass beads. The P designation indicates that the resultant substrate is modified PGC borosilicate glass beads, and the letters are used to indicate the surface modifying agent employed.

TABLE 5

| | | Surface Coverage |
|---|---|---|
| Sample | Surface Modifying Agent | (μmole/gm) |
| P-A | 3-aminopropyltrimethoxysilane | 4.05 |
| P-B | (3-trimethoxysilylpropyl)diethylenetriamine | 2.50 |
| P-D | N-trimethoxysilylpropyl-N,N,N- | ND |
| | trimethylammonium chloride | |

D. Preparation of 6.0 Micrometer Surface Modified Magnetic Particles

A suspension was prepared from 0.1 grams of 6.0-μm magnetic particles suspended in 1.9 ml of water purchased from Micromod Partikeltechnologie (Sicastar-M-CT), 0.5 mmole of the surface modifying agent, and 1.25 ml of isopropyl alcohol. The slurry was gently stirred for 16 hours. The particles were allowed to settle on a magnet and the liquid decanted. The following step was performed twice. An additional 4 ml of isopropyl alcohol was added to the particles, the new suspension was vigorously stirred for one minute, the particles were allowed to settle on a magnet, and the liquid decanted. The surface modified silica gel was dried in a vacuum oven at 50° C. overnight. The amount of surface modification was determined by thermogravimetric analysis. Table 6 lists the surface modifying agents employed and the resulting surface coverage determined for modified 6.0-μm magnetic particles. The S6 designation indicates that the resultant substrate is modified 6 μm magnetic beads from Sicastar, and the letters are used to indicate the surface modifying agent employed.

TABLE 6

| | | Surface Coverage |
|---|---|---|
| Sample | Surface Modifying Agent | (mmole/gm) |
| S6-A | 3-aminopropyltrimethoxysilane | 0.11 |
| S6-B | (3-trimethoxysilylpropyl)diethylenetriamine | 0.06 |
| S6-D | N-trimethoxysilylpropyl-N,N,N- | 0.09 |
| | trimethylammonium chloride | |

E. Preparation of 5.0 to 10.0 Micrometer Surface Modified Magnetic Particles

A suspension was prepared from 0.1 grams of 5.0- to 10.0-μm magnetic particles suspended in 3.2 ml of water purchased from CPG, Inc (MPG Uncoated), 0.5 mmole of the surface modifying agent, and 1.25 ml of isopropyl alcohol. The slurry was gently stirred for 16 hours. The particles were allowed to settle on a magnet and the liquid decanted. The following step was performed twice. An additional 4 ml of isopropyl alcohol was added to the particles, the new suspension was vigorously stirred for one minute, the particles were allowed to settle on a magnet, and the liquid decanted. The surface modified silica gel was dried in a vacuum oven at 50° C. overnight. The amount of surface modification was determined by thermogravimetric analysis. Table 7 lists the surface modifying agents employed and the resulting surface coverage determined for modified 5.0- to 10.0-μm magnetic particles. The M designation indicates that the resultant substrate is modified MPG beads, and the letters are used to indicate the surface modifying agent employed.

TABLE 7

| | | Surface Coverage |
|---|---|---|
| Sample | Surface Modifying Agent | (mmole/gm) |
| M-A | 3-aminopropyltrimethoxysilane | 0.11 |
| M-B | (3-trimethoxysilylpropyl)diethylenetriamine | 0.07 |
| M-D | N-trimethoxysilylpropyl-N,N,N- | 0.07 |
| | trimethylammonium chloride | |
| M-K | tetradecyldimethyl(3- | 0.11 |
| | trimethoxysilylpropyl)ammonium | |
| | chloride | |
| M-P | octadecyldimethyl(3- | 0.11 |
| | trimethoxysilylpropyl)ammonium | |
| | chloride | |
| M-X | N,N-didecyl-N-methyl-N-(3- | 0.08 |
| | trimethoxysilylpropyl)ammonium | |
| | chloride | |

Table 8 provides the chemical names for the surface modifying agents analyzed in more detail herein. The invention contemplates the coating of any substrate with one or more of these surface modifying agents, the use of coated substrates in the Affinity protocol (either alone or in combination with SNAP methodology), and the design of devices such as filters and cartridges with a layer containing a substrate modified with one or more of

TABLE 8
Surface Modifying Agents

| | |
|---|---|
| A | 3-aminopropyltrimethoxysilane |
| B | (3-trimethoxysilylpropyl)diethylenetriamine |
| C | N-(2-aminoethyl)-3-aminopropyltrimethoxysilane |
| D | N-trimethoxysilylpropyl-N,N,N-trimethylammonium |
| | chloride |
| E | bis(2-hydroxyethyl)-3-aminopropyltriethoxysilane |
| F | (N,N-dimethylaminopropyl)trimethoxysilane |
| G | N-(3-triethoxysilanepropyl)-4,5-dihydroimidazole |
| H | 2-(trimethoxysilylethyl)pyridine |
| I | (aminoethylaminomethyl)phenethyltrimethoxysilane |
| J | 2-(diphenylphospino)ethyltriethoxysilane |
| K | tetradecyldimethyl(3- |
| | trimethoxysilylpropyl)ammonium chloride |
| L | Diethylphosphatoethyltriethoxysilane |
| M | 3-mercaptopropyltrimethoxysilane |
| N | N-phenylaminopropyltrimethoxysilane |
| O | N-(6-aminohexyl)aminopropyltrimethoxysilane |
| P | octadecyldimethyl(3- |
| | trimethoxysilylpropyl)ammonium chloride |
| Q | N-(trimethoxysilylpropyl)isothiouronium chloride |
| R | N-(trimethoxysilylpropyl) ethylenediamine, triacetic |
| | acid, trisodium salt |
| S | N-(2-aminoethyl)-11-aminoundecyltrimethoxysilane |
| T | N-(3-triethoxysilanepropyl)gluconamide |
| U | N-(triethoxysilanepropyl)-O-polyethylene oxide |
| | urethane |
| V | 3-(trihydroxysilyl)-1-propanesulfonic acid |
| W | carboxyethylsilanetriol |
| X | N,N-didecyl-N-methyl-N-(3- |
| | trimethoxysilylpropyl)ammonium chloride |
| Y | 2- |
| | [methoxy(polyethyleneoxy)propyl]trimethoxysilane |

Furthermore, the chemical structures for each of surface modifying agents A-Y are provided in FIG. 10. We additionally note the following information regarding the formula weight of each of coupling agents A-Y, as well as a common abbreviation used to refer to each:

| Agent | Formula Weight | Abbreviation |
|---|---|---|
| A | 179.29 | AP |
| B | 265.43 | DETAP |
| C | 226.36 | AEP 3-1-1 |
| D | 257.83 | TMAP-Cl |
| E | 309.48 | BHOEAP |
| F | 207.34 | DMAP |
| G | 274.43 | DHIAzP 2-1-1 |
| H | 227.33 | PyrE |
| I | 298.46 | AEAMPhE |
| J | 376.50 | DPhPhoE |
| K | 440.18 | TDDMAP-Cl |
| L | 328.41 | DEPhaE 3-2-1 |
| M | 196.34 | MCP |
| N | 255.39 | PhAP |
| O | 278.47 | AHAP |
| P | 496.29 | ODDMAP-Cl |
| Q | 274.84 | PITU |
| R | 462.42 | EDTAP |
| S | 334.57 | AEAU |
| T | 399.51 | GAP |
| U | 400-500 | POPEOU |
| V | 202.26 | THOSPSA |
| W | 196.14 | COESTO |
| X | 510.32 | DDMAP-Cl |
| Y | 460-590 | MOPEOP |

F. Peptide-Based Surface Modifying Agents

In addition to the foregoing amine-based chemical functionalities, the present invention contemplates surface modifying agents composed in whole or in part of peptides. Such peptides can be attached to the surface of a substrate directly, via a cleavable linker, or via a chemical functionality which is itself directly appended to the surface of the substrate.

Exemplary peptides for use as surface modifying agents include any peptide that interacts with a target such that it increases the affinity of a coated substrate for that target. Specific examples of peptides suitable as surface modifying agents include the family of anti-microbial peptides, aptamers, and PNA. As with other types of substrates and substrate coatings, peptide-based surface modifying agents can be used to bind to any of a wide range of targets including DNA, RNA, protein, bacterial cells or spores (gram+ or gram−), viruses (DNA- or RNA-based), small organic molecules, and chemical compounds. Preferred peptide-based surface modifying agents will be relatively stable under the particular conditions required to promote interaction of the peptide-based coated substrate with the target.

### Example 4

### Cleavable Linkers for Releasing Active Region-Target Complexes from a Substrate

The following are non-limiting examples of methods that can be used to release active region-target complexes from the remainder of the surface modifying agent+substrate.

A. Fluoride labile alkylsilyl linker in coupling reaction An alkylsilyl moiety can be used in the coupling region to attach the surface modifying agent to the substrate. Following binding of target to the active region of the surface modifying agent, hydrofluoric acid can be employed to cleave the silicon-oxygen bond and detach the active region from the remainder of the surface modifying agent+substrate.

B. Fluoride Labile Alkylsilyl Linker in Spacer Region An alkylsilyl moiety can be used in the backbone of the spacer region that is used to attach the active region to the substrate. Following binding of target to the active region of the surface modifying agent, hydrofluoric acid can be employed to cleave the silicon-oxygen bond and detach the active region from the remainder of the surface modifying agent+substrate.

C. Acid Labile Carbonyl Linker in Spacer Region

An acid labile carbonyl moiety can be used in the backbone of the spacer region that is used to attach the active region to the substrate. Examples of acid labile carbonyl moieties are amides, esters, carbonates, urathanes, and ureas. Following binding of target to the active region of the surface modifying agent, acids such as trifluoracetic acid, hydrochloric acid, hydrobromic acid, nitric acid, phosphoric acid, and sulfuric acid can be employed to cleave the acid labile carbonyl moiety.

D. Base Labile Carbonyl Linker in Spacer Region

A base labile carbonyl moiety can be used in the backbone of the spacer region that is used to attach the active region to the substrate. Examples of base labile carbonyl moieties are amides, esters, carbonates, urathanes, and ureas. Following binding of target to the active region of the surface modifying agent, bases such as ammonium hydroxide, sodium hydroxide, and potassium hydroxide can be employed to cleave the base labile carbonyl moiety.

E. Nucleophile Labile Linker in Spacer Region

A nucleophile labile moiety can be used in the backbone of the spacer region that is used to attach the active region to the particle. An example of a nucleophile labile moiety is an oxime or a sulfonamide. Following binding of target to the active region of the surface modifying agent, any organic based amine can be employed as a nucleophile to effect cleavage.

F. Photo Labile Linker in Spacer Region

A photo labile moiety can be in the backbone of the spacer region which is used to attached the active region to the particle. Examples of photo labile moieties are esters, nitro substituted arylhydroxymethyl esters and arylsubstituted diazo derivatives. Following binding of target to the active region of the surface modifying agent, light can be employed to induce cleavage of the photo labile moiety. The wavelength of light employed is not critical, however the light will preferably have a wavelength of between 800 and 100 nm, with a more preferred wavelength between 465 and 190 nm, and a most preferred wavelength between 365 and 240 nm.

### Example 5

### Testing of Novel Surface Modified Beads

As described in detail above, we synthesized a variety of bead-shaped substrates modified with various amine-functionalized surface modifying agents. Coated beads were assessed for their interaction with doubled-stranded DNA, as well as for their interaction with bacterial cells and spores. The beads are referred to using letters A-P, and A-P refer to the same modification as presented in Table 8 above, except where otherwise noted (bead P corresponds to bead W-U). Specifically, the beads are the 50 μm silica gel beads described in Table 3 and indicated with a W.

FIG. 11 summarizes results indicating that several of the amine-functionalized substrates have improved adhesion for DNA (FIG. 11). For bead screening of DNA adhesion, 5 mg of 50 μm beads were added to a sample containing 200 ng of calf thymus dsDNA (target) in 1.5 mL dionized water at pH 5. The mixing time for adhesion is set for 5 min to enable reasonable processing times, though longer mixing times typically improved adhesion efficiency. Adhesion of double-stranded DNA to the beads was measured using the fluorescence detection methods described herein.

The conditions used to examine the adhesion efficiency of cells and spores to the beads were largely the same as that used to measure interaction with DNA. Briefly, 5 mg of beads were mixed with a sample of 10⁹ cells/mL in 1.5 mL water at pH 5 for 5 min. Samples with beads were mixed by slow rotation and the solution tested for fluorescence or using flow cytometry before and after the addition of beads. A decrease in the amount of target in the sample indicates better adhesion and thus more efficient capture. For the measurements of cell adhesion, absorbance measurements were also run to confirm results.

FIG. 12 summarizes the results of analysis of the interaction of two different bacterial cells (two different targets) with beads A-P and beads 1-11. Beads 1-11 correspond to the commercially available beads described in Table 2. Briefly, the various modified beads were analyzed for their ability to interact with bacterial cells from either B. anthracis (Ba) or B. thuriengensis (Btk).

FIG. 13 summarizes the results of analysis of the interaction of two additional bacterial cells (two different targets) with beads A-P and beads 1-11. Beads 1-11 correspond to the commercially available beads described in Table 2. Briefly, the various modified beads were analyzed for their ability to interact with bacterial cells from either E. coli or Y. pestis (Yp).

FIG. 14 summarizes the results of analysis of the interaction of beads A-P and beads 1-11 with either B. anthracis (Ba) cells (vegetative) or sporulated B. anthracis (Ba Spores). Beads 1-11 correspond to the commercially available beads described in Table 2, and the various modified beads were analyzed for their ability to interact with either the vegetative or sporulated form of B. anthracis (Ba).

FIG. 15 provides scanning electron microscope (SEM) images. These images were taken to demonstrate that cells (targets) physically adhere to the beads. Briefly, beads were incubated with samples containing B. anthracis vegetative cells or spores, and SEM images were taken to ascertain whether the cells and spores physically associated with the beads. As can be seen from examination of the SEM images, cells and spores adhered to the surface of the beads. We note, however, that the surface of the beads do not appear saturated with target even at high concentrations of ˜10⁹ cells or spores. In the case of vegetative Ba, the chains of bacteria can be observed to span several beads and cause them to clump together.

FIG. 16 demonstrates that analysis of a sample using both the Affinity Protocol and SNAP methodologies provides improved detection of bacterial target DNA in comparison to the use of SNAP technology alone.

### Example 6

### Factors that Influence Adhesion

An important goal of the methods of the present invention is the identification of parameters which will allow Affinity Protocol technology to be used under conditions that (a) can be easily employed in the field (e.g., at a crime scene, environmental site, accident scene, etc) and (b) are adaptable to a wide range of samples, substrates, and targets. Accordingly, we performed a series of experiments designed to understand the factors that influence DNA adhesion to substrates.

We examined the impact of a range of pH and salt concentrations on the interaction of beads coated with coating B (a triamine coating). Briefly, the experiments involved adjusting the pH and ionic strength of the sample solutions and measuring the corresponding effects on target capture and subsequent release from the beads. Both pH and ionic strength have a profound effect on the % efficiency of DNA adhesion to the beads.

FIGS. 17-18 summarize the results of experiments in which the interaction of double-stranded calf thymus DNA with a bead coated with coating B was examined. The interaction of DNA with the bead was influenced by the salt concentration and pH, and this interaction dropped off sharply between a salt concentration of 0-500 mM.

In a next set of experiments, we analyzed the interaction of beads coated with coating D with DNA seeded into samples of either water, bacterial culture supernatant, or non-laboratory-grade environmental water. FIG. 19 summarizes the results of these experiments, and indicates that the coated beads can efficiently bind target contained in a wide range of samples.

### Example 7

### Factors that Influence Target Release

Although the first step in evaluating the utility of a particular coated or uncoated substrate is determining the ability of that substrate to interact with a target, further analysis of the target likely requires the ability to recover the target from the substrate. Given the high level of sensitivity of many modern techniques for analyzing targets, it is not necessary for all of the target to be readily released from the substrate. However, the ability to recover an amount of target sufficient for further analysis is important.

As our previous analysis of the factors which influence DNA adhesion to a substrate indicated, adhesion (e.g., both adhesion and release of target) between substrate and target DNA is greatly influenced by pH and salt concentrations. Accordingly, methods which can be used to release target from a substrate include the manipulation of pH and salt concentration. Additionally, we found that temperature influences the adhesion of target DNA to a substrate (FIG. 20).

The invention contemplates that manipulation of any of a number of variables can be used to release target (DNA, RNA, protein, bacterial cells, etc) from a substrate. One of skill in the art can readily select from amongst these variables, and the optimal elution (e.g., release) conditions will vary based on the specific substrate employed, the specific target, the concentration of the target, and the initial adhesion conditions. Exemplary variables which can be manipulated include, without limitation: salt concentration (e.g., NaCl, CaCl₂, NaOH, KOH, LiBr, HCl), pH, the presence of spermidine, the presence of SDS, the type of buffer (e.g., carbonate buffer, Tris buffer, MOPS buffer, phosphate bugger), the presence of serum, the presence of detergents, the presence of alcohols, the time of adhesion, the temperature, and the application of mechanical agitation. Exemplary mechanical manipulations include sonication, use of a French press, electrical shock, microwaves, dehydration, vortexing, or application of a laser.

The invention further contemplates that the release of the target can be achieved by cleavage of a moiety that links the surface modifying agent to the substrate.

In still another embodiment, the invention contemplates the use of electroelution to recover target nucleic acid from a substrate.

Amine surface-functionalized beads have been developed and have been shown to exhibit a high affinity for DNA. The DETAP modified beads captured nucleic acids exceedingly well in a variety of liquid environments. However, although the high affinity for this substrate to DNA is desirable, it is equally desirable to be able to efficiently release target from the substrate so that the target can be further analyzed.

In addition to other methods for promoting release of targets from substrates, we have used an electric field to improve the efficiency of recovery of DETAP bead-bound DNA. Although the protocol currently being tested has not been efficient in recovering trace amounts of DNA from a substrate, this methodology has proved successful in releasing DNA when larger initial concentrations were adhered to the substrate.

Agarose and Calf Thymus DNA were purchased from Invitrogen (Carlsbad, Calif.). Agarose was melted in 0.5×TBE Electrophoresis Buffer (45 mM Tris-Borate, 1 mM EDTA). DETAP beads were synthesized, and the batch label PB-7 will be used to denote the amine-functionalized beads. GeneCapsule™ devices were obtained from Geno Technology (St. Louis, Mo.). Other standard reagents were of molecular biology grade purity.

Twenty PB-7 beads were loaded overnight in 1 mL water containing 50 μg/mL Calf Thymus DNA. Beads were loaded in a normal-mode 0.5% Agarose-TBE gel with 0.2 μg/mL Ethidium Bromide for visualization and covered with a top agarose containing 1N NaOH. Beads were also loaded in the GeneCapsule™ device using 0.5% Agarose-TBE containing various concentrations of NaOH. A 100 μL bed of agarose was set in the GelPICK™. Loaded beads were layered above this support bed, and an overlay of agarose was set. The GelTRAP™ was equilibrated in TBE for 15 minutes before the addition of 150 μL of fresh TBE and the insertion of the GelPICK™ to the level of the trap TBE as depicted in FIG. 21. Electrophoresis in both experimental setups was conducted at 200 V for 15 minutes with an additional three 5 second pulses at inverted polarity to liberate DNA from the GeneTRAP™ membrane. Eluate from the GeneCapsule™ was removed by puncturing the Collection Port and removing liquid by pipette.

All low DNA load experiments were conducted with the GeneCapsule™ device with 0.5% Agarose-TBE containing either 0.1N NaOH or 0.1N NaOH plus 100 μg/mL Calf Thymus DNA. Sets of twenty PB-7 beads were loaded for 30 minutes in 1 mL water containing 5, 50, or 500 pg/mL pCR2.1Topo-BtkCryIA Bacillus thuringiensis subspecies kurstaki gene copy standard plasmid. As above, loaded beads were layered above a 100 μL support gel in the GelPICK™, and an approximately 450 μL agarose overlay was set to fill the remaining volume. Pre-equilibrated GeneTRAPs™ were filled with 150 μL fresh TBE, the loaded GelPICK™ was inserted. Electrophoresis of the loaded GeneCapsules™ was conducted at 200V for either 15 minutes or 45 minutes. Eluates were removed through the pierced Collection Port via pipette. Control samples were eluted by incubation in 150 μL of 0.01N NaOH plus 100 μg/mL Calf Thymus DNA for 15 minutes at room temperature. Samples were assayed by TaqMan® real-time PCR.

As indicated by the gel presented in FIG. 21, high DNA loads can be efficiently recovered using electroelution. FIG. 21C shows a load of 50 μg of Calf Thymus DNA easily migrating away from beads when exposed to an electric field.

Initially we note that our experiments indicate that DNA could be separated from the amine beads with relatively low voltages (˜10 V/cm within 15 minutes). The table below summarizes the results obtained using several low voltage electroelution to release DNA from a substrate. We note that under conditions of varying salt concentrations, the yield of DNA is good, however, the highest recovery was observed under higher NaOH concentration (e.g., a more alkaline environment).

| Beads | Agarose | NaOH | Captured | Recovered | % Recovered |
|---|---|---|---|---|---|
| No Beads | 0.5% | 0.00 N | 50 μg | 50 μg | 100% |
| PB-7 | 0.5% | 0.00 N | 24 μg | 2 μg | 8% |
| PB-7 | 0.5% | 0.01 N | 28 μg | 1 μg | 4% |
| PB-7 | 0.5% | 0.10 N | 20 μg | 9 μg | 45% |

These experiments indicate that electroelution is another mechanism that can be used to release target from a substrate. The present conditions have not been optimized for very low concentrations of DNA, however, the results indicate that electroelution represents a quick, safe, and cost-effective mechanism for releasing target from substrate.

### Example 8

### The Use of Cleavable Linkers to Release Target from a Substrate

As outlined in detail above, an important aspect of the invention is the ability to release target from the substrate so that the target can be further analyzed. One mechanism that can facilitate the release of target from substrate is the use of surface modifying agents containing cleavable linker that can be specifically cleaved to release target from substrate. The invention contemplates the use of any of a number of cleavable linkers.

One possible concern with the use of cleavable linkers is that the agents needed to induce cleavage of the linker may either degrade the target or may otherwise inhibit the further analysis of the target. To address this possible concern, we analyzed target DNA in the presence of DETAP or the cleavage product DETA to evaluate a possible inhibitory role for these moieties in further molecular analysis of the DNA by PCR. Based on our analysis, we concluded the presence of DETAP, and the cleavage product DETA, does not prevent further analysis of DNA by real-time PCR.

Briefly, Diethylenetriamine and (3-trimethoxysilyl-propyl)-diethylenetriamine were obtained from Sigma-Aldrich (DETA 103.2 g/mol, 0.95 g/mL; DETAP 265.4 g/mol, 1.031 g/mL). Serial dilutions of each were made in autoclaved diethylpyrocarbonate-treated water from Ambion.

Target DNA was either crude plasmid DNA from Bacillus thuriengensis subspecies kurstaki or the gene copy standard pCR2.1Topo-BtkCryIA. TaqMan® real-time PCR chemistry was used to assay samples on the ABI 7700 Sequence Detection System.

TaqMan® real-time PCR assays were performed in a standard 50 μL volume. Except for negative controls, assay reagent was spiked with 50 pg/mL of target DNA. Samples were spiked with varying concentrations of either DETAP or DETA, and water was added to the positive controls.

Inhibition of PCR was measured as a change in threshold cycle relative to the threshold cycle of the positive control containing no amine additive. Percent inhibition was taken as the ratio of the change in threshold cycle to the threshold cycle of the positive control. Our result indicated that DETAP can be inhibitory to PCR at higher concentrations. However, at concentration relevant to the application of bead-based DNA capture and release (˜25 nmol amine functionality), the level of inhibition drops significantly. The addition of 20 nmol of DETAP to a 50 μL PCR reaction results in a threshold cycle shift of approximately 2 (˜9% inhibition of signal).

In contrast, our results indicated that DETA alone does not significantly inhibit PCR. At both quantities relevant to the bead-based assay and at quantities that are several orders of magnitude greater, there is no apparent shift in threshold cycles due to the DETA additive relative to positive controls.

These results indicate that the use of surface modifying agents containing cleavable linkers is a feasible approach for facilitating the substrate based capture of targets, the release of those targets, and the further molecular analysis of those targets.

A second class of cleavable linkers that can be used to reversibly attach surface modifying agents to substrate are ammonia labile linkers. Accordingly, in a second set of experiments, we analyzed whether ammonia inhibits the further analysis of target DNA by PCR.

Two experiments were performed. The target was supernatant from vegetative Ba grown in BHI (culture medium) overnight, and centrifuged for 5 minutes at 3000 rpm to pellet the cells. Supernatant dilutions were prepared in BHI.

Various concentrations of ammonia were mixed with various dilutions of Ba supernatant, and allowed to incubate at room temperature. The resulting mixture was used as the eluate in a standard TaqMan reaction in the ABI7700. 5 μL of each eluate (out of a total of 50 μL) was added to the PCR reaction well, with the Ba primer-probe set. All samples were prepared in duplicate. Controls consisted of supernatant dilution (in the absence of ammonia) placed directly into the PCR well.

The results of two independent sets of experiments demonstrated that the addition of ammonia can be sustained up to a level of 0.005M concentration in the PCR reaction without any loss of PCR efficiency. Even at an ammonia concentration of 0.05M, a loss of PCR efficiency of only approximately 1-2 orders of magnitude was observed. Additionally, our observations indicated that low levels of ammonia may actually improve the efficiency of the PCR reaction—perhaps due to a favorable change in the pH of the PCR reaction mix.

### Example 9

### Optimization of Target Capture and Release

The Affinity Protocol is broadly applicable to identifying and/or separating any of a number of targets from amongst heterogeneous liquid and solid samples. Even in a relatively unoptimized form, the Affinity Protocol provides increased sensitivity for detecting small concentrations of target from a heterogeneous sample, and thus even an unoptimized form of the protocol has substantial benefits in a variety of settings. However, further optimization of the Affinity Protocol has a variety of additional benefits including, but not limited to (i) the ability to detect a smaller concentration of target, (ii) the ability to identify and/or separate target in less time, (iii) the ability to detect capture upon the substrate of a higher percentage of the available target within a sample, (iv) the ability to release/elute from the substrate (e.g., for further analysis or separation) a higher percentage of the bound target, and (v) the ability to perform the Affinity Protocol using fewer starting materials (e.g., fewer consumables, less substrate).

The following examples detail experiments conducted to optimize the Affinity Protocol, and to thus achieve some of the benefits outlined above.

(a) Capture and Elution Efficiencies of Coated Substrates.

We tested several commercially available and laboratory-synthesized coated substrates to access the efficiency with which each coated substrate captured and released target. In this particular example, the target was DNA and the substrates were various magnetic beads modified with a surface modifying agent.

The following commercially available beads were used: Cortex-Biochem polystyrene-amine beads, Dynal M-270 polystyrene-amine beads, Polysciences polystyrene beads, Biosource silanized FeO-amine beads, and streptavidin functionalized beads. Additionally, the following laboratory-synthesized beads were used: M-B-1, M-B-2, and M-B-3. The laboratory synthesized beads were made as follows: 5-10 μm of uncoated magnetic particles (aka—beads of 5-10 μm particle size or beads of 5-10 μm in diameter; obtained from CPG, Inc.) were suspended in a combination of water, the surface modifying agent, and isopropyl alcohol. This slurry was gently stirred for 16 hours. The particles were allowed to settle on a magnet, and the liquid was decanted. The following was repeated two times. Additional isopropyl alcohol was added to the particles, the suspension was stirred vigorously for one minute, the particles were allowed to settle on a magnet, and the liquid was decanted. The surface-modified silica beads were dried in a vacuum overnight at 50° C., and following drying, the amount of surface modification was determined by thermogravimetric analysis.

FIG. 22 summarizes a series of experiments conducted using beads M-B-1, M-B-2, M-B-3, as well as the commercially available beads. These experiments examined the capture and release activity of each coated, magnetic bead using a DNA target. Briefly, one milligram of coated beads were added to 1 mL of 500 pg/mL DNA. The efficiency with which the beads captured the DNA was measured, and is represented by the left-most bars in FIG. 22. The efficiency with which the DNA was released (e.g., eluted) from the beads was measured. The elution efficiency is referred to interchangeably as the percentage recovery, and is represented by the middle bars in FIG. 22. DNA was released into an elution buffer including 150 μL of 100 μg/mL calf-thymus DNA in 0.01N NaOH. The ratio of recovered DNA to captured DNA is the elution efficiency. Finally, the percentage efficiency of each bead was analyzed and is represented by the right-most bars in FIG. 22. The percentage efficiency is the ratio of the recovered DNA to the total amount of target DNA in the starting sample (500 pg in this example).

In certain embodiments, the invention contemplates capture efficiencies of greater than 75%, 80%, 85%, 90%, 95%, 96%, 97%, 98%, or greater than 99%. In certain other embodiments, the invention contemplates capture efficiencies of 100%.

In certain embodiments, the invention contemplates elution efficiencies of greater than 75%, 80%, 85%, 90%, 95%, 96%, 97%, 98%, or greater than 99%. In certain other embodiments, the invention contemplates elution efficiencies of 100%.

In any of the foregoing, the invention contemplates an overall efficiency of greater than 75%, 80%, 85%, 90%, 95%, 96%, 97%, 98%, or greater than 99%. In certain other embodiments, the invention contemplates an overall efficiency of 100%.

(b) Substrate Quantity and Capture Time

The Affinity Protocol is suitable for a number of applications. Many of these applications are sensitive to cost, time, and the amount of consumable supplies required to conduct the method. Accordingly, we performed a number of experiments to examine capture efficiency as a function of the amount of substrate and the capture time (e.g., the amount of time allotted for substrate-sample interaction). The results of these experiments are summarized graphically in FIGS. 23 and 24. Briefly, commercially available, amine coated magnetic beads (Dynal) were used to capture a DNA target from 1 mL of bacterial culture supernatant diluted in water. The concentration of substrate was varied between 1 mg and 5 mg, and the capture time was varied between 1 minute and 10 minutes.

We note that as little as 1 mg of substrate (e.g., beads) for 1 minute is sufficient to capture greater than 90% of the target in this sample. Increasing the substrate concentration, the capture time, or both increased the capture efficiency to greater than 99.99%. One can manipulate these parameters depending on the requirements of the particular application of the Affinity Protocol to arrive at the appropriate combination of efficiency and cost.

(c) Substrate Quantity and Elution Time

As outlined in detail above, for many of the possible applications of the Affinity Protocol, the total amount of time required to perform the method is an important factor. Accordingly, we examined the elution efficiency as a function of both substrate quantity and elution time. The results of these experiments are summarized graphically in FIGS. 25 and 26. Briefly, commercially available, amine coated magnetic beads (Dynal) were used to capture a DNA target from 1 mL of bacterial culture supernatant diluted in water. The elution was performed in elution buffer including 150 μL of 100 μg/mL calf thymus DNA in 0.01N NaOH. The concentration of substrate was varied between 1 mg and 5 mg, and the elution time was varied between 1 minute and 10 minutes. We note that there was no significant change in elution efficiency across these concentrations of substrate and elution times.

(d) Elution Volume

As outlined in detail above, for many of the possible applications of the Affinity Protocol, the amount of reagents required to perform the method is an important factor. The need for reagents not only increases the cost of the method, but also increases the amount of materials that must be transported and maintained in the field for applications of the invention that are not conducted in a traditional laboratory setting. One of the possible reagents required for the Affinity Protocol is the elution buffer needed to recover captured target from the substrate. Accordingly, we examined the effect of elution buffer volume on elution efficiency.

The results of these experiments are summarized in FIG. 27. Briefly, target was eluted following capture from a 5 mL sample in elution buffer including 150 μL of 100 μg/mL of calf thymus DNA in 0.01N NaOH. The elution buffer volume was varied from 1 mL to 150 μL. No significant change in elution efficiency was observed across this range of elution buffer volume. Accordingly elution buffer volume can be chosen based on the particular requirements of the application of the Affinity Protocol.

In certain embodiments, the method of eluting target from substrate is performed in a volume of elution buffer less than 1/5th the volume of the initial sample from which the target was captured. In certain other embodiments, the method of eluting target from substrate is performed in a volume of elution buffer less than 1/6th, 1/7th, 1/8th, 1/9th, 1/10th, 1/15th, 1/20th, or 1/25th the volume of the initial sample from which the target was captured. In certain other embodiments, the method of eluting target from substrate is performed in a volume of elution buffer less than 1/30th, 1/40th, or 1/50th the volume of the initial sample from which the target was captured.

(e) Elution pH

The standard elution buffer used in these experiments (100 μg/mL of calf thymus DNA in 0.01N NaOH) has a pH of 11.8. We examined the effect on elution efficiency of small changes in the pH of the elution buffer. The results of these experiments are summarized in FIG. 28. Briefly, we found that variations in the pH of the elution buffer between approximately pH 11.5-12.3 had no statistically significant impact on elution efficiency.

(f) Elution Buffer Optimization

As outlined in detail above, calf thymus DNA was included in the elution buffer. Accordingly, we conducted experiments to assess whether elution efficiency was sensitive to the concentration of calf thymus DNA included in the buffer. Briefly, we varied the concentration of calf thymus DNA in the elution buffer between 50 μg/mL and 500 μg/mL. We observed no significant increase in elution efficiency with concentrations of calf thymus DNA greater than 100 μg/mL. Thus, we selected a standard concentration of 100 μg/mL of calf thymus DNA for use in the elution buffer given that the use of additional reagent (e.g., with the concomitant expense) produced no significant benefit with respect to elution efficiency.

(g) Washing

One or more wash steps are typically employed in many isolation or separation protocols. Accordingly, one embodiment of the Affinity Protocol could involve a wash step following target capture but prior to target release. Such a wash step could be used to remove low affinity materials from the substrate, and to thus increase the specific capture and elution of target that binds with increased affinity to the substrate. However, the need for one or more wash steps increases the time, cost, and amount of reagents necessary to perform the Affinity Protocol. Accordingly, we conducted a series of experiments to assess the need for one or more wash steps following target capture but prior to target elution.

Briefly, we performed the Affinity Protocol in the presence or absence of two 1 mL wash steps. The results of these experiments indicated that the wash steps were not required and, in fact, did not significantly altered the efficiency of DNA recovery. Additional experiments performed using DNA suspended in other, more heterogeneous sample such as growth media or non-laboratory water indicated that wash steps were not necessary. We note that the presence of two wash steps did not significantly decrease the efficiency of DNA recovery, and thus wash steps could be employed if necessary or desired in certain applications. For example, if the sample is extremely heterogeneous, hazardous, or contains a high concentration of inhibitory materials that may effect further analysis of isolated target, then wash steps can be employed without a significant negative effect on recovery efficiency. If, on the other hand, speed or cost are significant issues, the post-capture wash step can be omitted.

### Example 10

### Rapid Affinity Protocol

The Affinity Protocol provides an improved method for separating and/or identifying a target from a heterogeneous sample using a substrate. The substrates can be of virtually any size or shape, can be magnetic or non-magnetic, and can be modified with one or more surface modifying agents that preferentially increase the affinity for the modified substrate to a particular target in comparison to the affinity of the modified agent for other material in the sample.

The Affinity Protocol is suitable for any of a large number of laboratory or field applications. Furthermore, as outlined in detail in Example 9, aspects of the Affinity Protocol can be manipulated to (i) decrease the time required to perform the method, (ii) decrease the cost of the materials required to perform the method, and (iii) decrease the number of materials required to perform the method. For example, the Affinity Protocol can be performed in a range of sample volumes, for example, 1 mL-5 mL. The Affinity Protocol can be performed using a range of substrate concentration, for example, 1 mg/mL-5 mg/mL of a substrate such as beads. The Affinity Protocol can be performed with a capture time of 5 minutes, or even less than 5 minutes, and with an elution time of 1 minute, less than one minute, or thirty seconds. Of course, one of skill in the art will readily appreciate that the present invention contemplates the use of any of a number of parameters, and the foregoing are merely indicative of parameters that can be advantageously used to decrease time and cost of carrying out this method.

We provide in detail herein a rapid application of the Affinity Protocol that was used to separate target from a heterogeneous sample. In this example, the total time required to separate target is less than 5 minutes. In this example, the substrate was 2.7 μm, amine derivatized, magnetic beads (Dynal), the target was DNA, and the sample was bacterial supernatant diluted in deionized, laboratory water. Below we have provided an exemplary, rapid protocol. Beside each step both the time required to conduct each step of the protocol and the total time elapsed is provided.

Protocol

| | | Step time | Total time |
|---|---|---|---|
| | Step | min:sec | min:sec |
| 1. | Pipette 33 μL of substrate into a 1.5 mL microcentrifuge tube. | 0:30 | 0:30 |
| 2. | Add 1 mL of liquid sample. Close the tube. | 0:30 | 1:00 |
| 3. | Vortex the tube for at least two seconds to distribute the beads | 0:45 | 1:45 |
| | throughout the sample. Place tube in a non-magnetic rack and allow it | | |
| | to sit for 30 seconds (capture time can be increased for trace level | | |
| | detection). | | |
| 4. | Open the tube and place in a magnetic separation rack if available, or | 0:15 | 2:00 |
| | use a standalone magnet to attract the beads to the side of the tube. | | |
| 5. | After the beads have moved to the side of the tube (approximately 10 | 0:15 | 2:15 |
| | seconds,) remove the fluid from the tube either by inverting the tube | | |
| | over a waste container and pipetting out the remainder or by pipetting | | |
| | out all of the fluid. Be sure to keep the tube in contact with the magnet | | |
| | during this process to avoid removing the beads. | | |
| 6. | Remove the tube from storage if necessary and place in a non-magnetic | 0:15 | 2:30 |
| | rack. | | |
| 7. | Add 150 μL of elution buffer (100 μg/mL calf thymus DNA in .01N | 0:30 | 3:00 |
| | NaOH pH = 11.8) | | |
| 8. | Close the tube and vortex for at least two seconds to expose all of the | 0:45 | 3:45 |
| | beads to the elution buffer. Place the tube in a non-magnetic rack and | | |
| | allow it to sit for 30 seconds. | | |
| 9. | Open the tube and place in a magnetic separation rack if available or use | 0:15 | 4:00 |
| | a standalone magnet to attract the beads to the side of the tube. | | |
| 10. | Pipette required quantity of fluid directly into PCR reaction tube or | 0:15 | 4:15 |
| | plate, or otherwise process for further analysis (if required). | | |

### Example 11

### Storage of Target

One application of the methods, compositions, and apparatuses of the present invention is for long term storage of targets separated from a sample. Such long term storage is useful in a variety of contexts. For example, efficient and reliable long term storage is useful in a forensic context for cataloging biological evidence. Furthermore, long term storage is useful in a medical context for preservation of samples for educational purposes, as well as preservation of samples for analysis that cannot be performed immediately upon target collection. Furthermore, long term storage is useful in a variety of environmental contexts where target collection may take place in the field but where target analysis will occur in a laboratory that may be geographically separated from the field site.

One example of long term storage involves the use of the substrate itself as a vehicle for the target. For example, following target capture on the substrate, the target-substrate complex can be separated from the sample, vacuum dried, and stored. This can be done extremely rapidly. In the rapid protocol summarized above, this drying and storage step may be optionally inserted following step 5 (e.g., following approximately 2 minutes of handling time). By way of specific example, the tube containing target-bead complex can be placed in a vacuum oven at 80° C. for approximately 30 minutes or until the bead pellet is dry. The dried pellet can be stored, for example, in a dark container with dessicant.

### Example 12

### Target Recovery from Complex Samples

As outlined in detail above, the Affinity Protocol can be effectively used to separate target from a sample. We have additionally tested the particular bead, capture, and elution conditions described in detail in Example 9 to assess the efficiency of target recovery from more complex samples. These more complex samples may more accurately mimic the types of medical and environmental samples to which this technology applies. Exemplary complex samples include solid samples such as soil, mud, clay, and sand or other high humic soils. Further exemplary complex samples include biological samples such as blood, urine, feces, semen, vaginal fluid, bone marrow, and cerebrospinal fluid. Still further exemplary complex samples include sea water, pond water, oil, liquid or solid mineral deposits, and dry or wet food ingredients.

Briefly, we separated target DNA from a number of complex samples using the Affinity Protocol. Separated target DNA was amplified using PCR. Our results indicated that target DNA could be separated from a complex sample using the Affinity Protocol, and that the separation was sufficient to remove agents that might inhibit PCR. Target DNA from both B. anthracis (Ba) and B. thuringiensis (Btk) culture supernatant was efficiently separated from non-laboratory grade, environmental water containing any of a number of complex contaminants not found in laboratory-grade water. Not only was the DNA efficiently captured and eluted, but it was also separated from inhibitory contaminants sufficiently to allow amplification of the DNA in a PCR reaction.

In a second set of experiments, target DNA from both B. anthracis (Ba) and B. thuringiensis (Btk) culture supernatant was efficiently separated from concentrated growth media (BHI) which contains any of a number of complex additives not found in laboratory or non-laboratory grade water. Not only was the DNA efficiently captured and eluted, but it was also separated from inhibitory contaminants sufficiently to allow amplification of the DNA in a PCR reaction.

In a third set of experiments, we separated target bacterial cells from complex samples using the Affinity Protocol. Briefly, we separated target DNA from a number of complex samples using the Affinity Protocol. DNA from separated target cells was amplified using PCR. Our results indicated that bacterial cells could be efficiently separated from complex samples, and furthermore that DNA from these bacterial cells could then be amplified by PCR. Ba, Btk, and Yp vegetative cells were used as target bacterial cells, and these targets were separated from non-laboratory grade, environmental water containing any of a number of complex contaminants not found in laboratory-grade water.

### Example 13

### Application of the Affinity Protocol to Dry Samples

As detailed herein, the affinity protocol can be used to separate a wide range of targets from various samples including gaseous, liquid, and solid samples. We now demonstrate that the separation of targets from various types of samples does not require that the samples first be rehydrated in water or otherwise processed to form a slurry. Although the rehydration of certain types of samples may be useful, certain materials such as clay soils are either difficult to rehydrate or become difficult to process further following their rehydration.

Dry biological particles typically carry a charge, and this charge can be used to help facilitate the separation of targets from dry samples such as soil samples or air. To more particularly illustrate, a magnetic substrate or a magnetic substrate coated with a surface modifying agent would be added to a sample and the sample and substrate would then be mixed so that the substrate contacts the sample. Following mixing, a target-substrate complex forms, and this can be processed using any of a number of methods detailed herein for examining targets separated by the Affinity Protocol.

FIG. 29 summarizes the results of an experiment conducted to illustrate that targets can be efficiently identified from dry samples. We seeded dry soil samples with a bacterial target. PCR analysis was performed on DNA isolated from the bacterial target using SNAP alone and compared to DNA isolated from the bacterial target using a combination of the dry affinity protocol and SNAP. In this experiment, the affinity protocol involved contacting the soil sample with electrostatically charged non-magnetic beads to concentrate the target prior to isolation of DNA using SNAP and PCR analysis. FIG. 29 shows that the use of the dry affinity protocol prior to DNA isolation and PCR can increase the relative signal in comparison with analysis of the soil sample in the absence of the affinity protocol. Such an increase in signal indicates (a) the dry affinity protocol can be used to separate target from dry samples and (b) the use of the affinity protocol provides improved detection of targets from a variety of samples including dry sample.

### Example 14

### Application of the Affinity Protocol to Dry Samples

Application of the Affinity Protocol to non-liquid samples has a variety of important environmental, medical, industrial, and safety applications. As outlined above, separation of target from dry sample can be accomplished by first rehydrating the dry sample to create a slurry which is then contacted with substrate to form target-substrate complexes that can be separated, and optionally analyzed further. Alternatively, separation of target from dry sample can be accomplished without the need to first rehydrate the dry sample.

We conducted additional experiments to separate and optionally analyze target from dry samples. In these experiments, cartridges comprising surface modified, magnetic substrates were used to perform the Affinity Protocol on dry samples. Briefly, Ba spores (target) were seeded at varying dilutions (0-10⁶ spores/mL of sand) into samples of sand. Each cartridge was loaded with 1 gram of sand wetted with 5 mL of distilled water. 15 mg (3 mg/mL) of magnetic beads (substrate) were used in the cartridge to capture the target. Capture time in this application of the Affinity Protocol was 5 minutes, and elution time was 1 minute.

Following elution of the target spores, DNA from the target was analyzed by PCR to assess the limit of detection of target in sand using the Affinity Protocol prior to PCR analysis, in comparison to the limits of detection using PCR alone. FIG. 30 summarizes the results of these experiments. We note that use of target separation using the Affinity Protocol resulted in an improvement in detection of the target of one order of magnitude in comparison to detection via PCR alone. Specifically, we detected DNA from bacterial spores in sand at a concentration as low as 100 spores/mL.

We note that this cartridge containing magnetic beads (the substrate) was similarly used effectively to perform the Affinity Protocol on other samples containing target. For example, this cartridge was used to separate bacterial cells or bacterial spores from non-laboratory grade, environmental water. Using substrate concentrations of 3 mg substrate/mL of sample, target capture times of 5 minutes, and target elution times of 1 minute, we observed one order of magnitude or greater improvements in detection in comparison to PCR alone. Specifically, we detected concentrations of bacterial cells and bacterial spores as low as 10 cells/mL of sample.

### Example 15

### Design and Use of a Chaotic Mixing Device

As outlined in detail above, the large-scale application of the Affinity Protocol and the Affinity Magnet Protocol may be facilitated by the development of devices which promote the efficient mixing of substrate and target within a large sample. We have constructed an apparatus to achieve journal bearing flow based on the principles outlined in FIG. 6. The apparatus is known herein as a Chaotic Mixing device or a Class I device, and one example of such an apparatus is shown in FIG. 31. The device shown in FIG. 31 consists of two Teflon cylinders, each of which is free to rotate about its central axis by means of a motor. The smaller cylinder is solid and placed eccentrically inside the larger cylinder. The sample is placed in the annulus between the two cylinders, and mixed by having both cylinders rotate simultaneously at 16 rotations per minute. The slow rotation rate maximizes diffusive mixing between the streamlines formed by stretching and folding the sample slurry. In certain embodiments using this device, the smaller cylinder was removed following mixing of substrate and target, and then replaced with an electromagnet. The electromagnet was then used to collect substrate-target complexes from the sample. In this particular example, the substrate was magnetic beads, and the electromagnet was used to efficiently collect magnetic beads.

We have used the Chaotic mixing device with the Affinity Protocol to extract bacterial targets from various types of soil, in quantities of 2 grams per sample. The large scale application of the affinity protocol demonstrates that these methods and devices are suitable for not only small sample sizes, but can also be scaled-up for industrial applications. The ability to scale-up the Affinity Protocol has implications not only for industrial applications of this technology. The results provided herein also demonstrate that certain target-substrate interactions may be more readily detected in larger volumes.

FIGS. 32 and 33 show the results of gel electrophoresis of DNA extracted using the Large-scale Affinity Protocol (Affinity Protocol carried out in a Chaotic mixing device) plus SNAP, in comparison to the use of SNAP alone in a smaller volume. Briefly, particular soil samples were analyzed using either the SNAP protocol or the Large-scale Affinity Protocol plus SNAP, and isolated target DNA was amplified by PCR. In this particular example, the substrate was uncoated magnetic beads. As can be seen from the results provided in FIGS. 32 and 33, the use of the large-scale affinity protocol resulted in an improvement in the limit of detection in certain soil types. Specifically, in a sludge sample, we were able to improve the detection limit by one order of magnitude, and in the Cary soil type (containing a high level of humic acids, a known PCR inhibitor) we were able to obtain detection where none was possible with SNAP processing only.

### Example 16

### Alternative Devices

As outlined in detail herein, the present invention contemplates that a wide range of substrates can be used in the Affinity Protocol. Such substrates may be further coated with one or more surface modifying agents. One example of an alternative substrate that can be coated with one or more surface modifying agents is provided in FIG. 34. FIG. 34 shows a functionalized substrate that would be useful in a wide range of applications. In this example, the inner walls of a centrifuge or PCR tube (where X=one or more surface modifying agents).

The use of functionalized tubes and culture vessels would help eliminate sample transfer—which would reduce both possible error and contamination, and reduce the need for additional supplies. Additionally, the use of such substrates would allow the target adhesion and further analysis to occur in a single vessel, and is thus readily adaptable to field applications or other settings where supplies and time may be limiting.

Other specific devices that can be designed based on the Affinity Protocol described herein are devices which facilitate gaseous or liquid sample collection and analysis. These devices will be broadly referred to as Class 2 devices. The invention contemplate the construction of both wet and dry filters. The filters can contain one or more layers of substrate (e.g., beads, paper, etc). Dry or wet samples that pass over/through the filter will pass through the substrate, and target within the sample will adhere to the substrate. FIG. 35 provides illustrations of representative filters that can be used to detect targets in air or water sample.

By way of further example of a dry format filter, one or more layers of substrate such as beads can be packed. The invention contemplates filters containing multiple layers of either the same substrate or of different substrates, as well as filters containing a single layer. In embodiments where the filter contains a single layer, the layer may contain a single substrate, a single substrate derivatized with multiple surface modifying agents, or multiple substrates. Air flows through the filter, and targets in the air sample are adsorbed onto the beads.

The invention contemplates the use of these filters alone, or in combination with other air filters commonly used in buildings and vehicles. For example, an Affinity Protocol-based filter can be added to a buildings HVAC system to provide a means for further analyzing the quality of the air circulating in the building.

Similarly, wet-filters can be used to assess the presence of targets in water samples. Such filters can be used to monitor reservoirs and thus assess the quality of drinking water, to monitor lakes or ponds and thus assess the health of these environments. These filters can be modified for use in aquariums, and thus help to both evaluate the quality of the water and to diagnose any water-related problems. Furthermore, these filters can be used in the home in combination with commercially available water purification devices. The invention contemplates the use of these filters alone, or in combination with other water filters commonly used in home, environmental or industrial applications.

The invention further contemplates the construction of another class 2 device: Affinity Protocol cartridges. These particular cartridges were designed based on cartridges previously designed and disclosed in US publication no. 2003/0129614 (U.S. patent application Ser. No. 10/193,742, hereby incorporated by reference in its entirety), however, the present invention contemplates cartridges that contain only a means for performing the Affinity Protocol on a sample, as well as cartridges that contain both a mean for performing the Affinity Protocol and a means for performing the SNAP protocol.

The following device, used for the collection and purification of an environmental, clinical, bioagent, or forensic sample containing DNA, was described in US publication no. 2003/0129614. This device can be further modified to include a means for performing the Affinity Protocol on a sample.

FIG. 36 provides a brief summary of the device. The device consists of two parts, an outer container and an inner housing. The inner housing contains a porous substrate that provides the functions of purification of the DNA and retention of inhibitors to PCR (polymerase chain reaction), used to amplify the extracted DNA (e.g., this porous substrate provides a means for performing the SNAP method on a sample). The outer container can serve a dual purpose, depending on the manner in which it is prepared, as indicated in FIG. 36. When used for storage and transport, the outer container includes a desiccant for enhancing drying of the porous substrate after the sample has been applied to it. The desiccant is separated from the porous substrate by means of a ring, such that the porous substrate does not touch the desiccant. When used for processing of the sample collected on the porous substrate, the outer container is sealed with a heat-sealable membrane, and contains liquid used to elute the DNA. The sample is processed by removing the heat-sealable membrane and pushing the inner housing into the outer cylinder, causing the liquid to flow through the porous substrate and carry the DNA into the resulting eluate.

The outer container can be attached to the inner housing by means of a tether and screw or snap fastener on the bottom of the outer container. The outer container can also have a flange integrated into the bottom surface, to provide stability and prevent tipping when the cylinder is resting on a surface.

In one modification of this device, an additional layer is introduced such that sample is brought into contact with a means for performing the Affinity Protocol (e.g., a substrate that binds to target) prior to being brought into contact with the SNAP filter.

Another possible modification of the device involves the addition of processing steps after the purification and inhibitor binding steps described earlier. It is well-known that under the appropriate salt and pH conditions, nucleic acid will bind strongly to silica and glass, while other classes of compounds will not be as strongly bound (for example, see Tian et al. 2000 Analytical Biochemistry, 283:175-191). By changing the pH and/or salt conditions, the nucleic acid can be eluted from the silica/glass material, thus allowing selective binding and subsequent release of nucleic acid from a mixed sample. This effect, described in the “Boom” patent U.S. Pat. No. 5,234,809, is the basis of several existing commercial nucleic acid purification technologies, produced by companies such as Qiagen and Promega. We provide a novel implementation of this “Boom” effect that is mechanically and chemically compatible with our devices and can further facilitate the detection and analysis of target within a sample.

The processing of the sample with the device proceeds as described earlier up to the point at which it is brought into contact with a chaotropic salt on a solid matrix and eluted from that matrix. At this point in the process, the sample contains high concentrations of chaotropic salt, which promotes binding of nucleic acid to silica or glass. The sample is next brought into contact with a silica or fused glass substrate. In a preferred embodiment, the sample is eluted through a silica column by applying positive pressure with a plunger (see FIG. 37). As the sample passes over the silica column, nucleic acids are bound to the column. The fluid continues past the silica column into an absorbent material that captures and retains the sample fluid. The silica column can be constructed in a “slider” format which allows the user to easily transfer the silica column into a second chamber by pulling the slider. In one embodiment, the act of pulling the slider acts to open a buffer reservoir in the second chamber. In FIG. 37, the second, low-salt, buffer reservoir is opened and the liquid forced through the silica column by the user applying pressure with a second plunger, thus eluting the nucleic acid into a clean compartment. Access to this sample can be through any one of a number of modes, including a septum, a threaded plug, or an integrated syringe. The orientation of the second chamber relative to the first chamber can be rotated 180°; that is, the two plungers can be either side-by-side or on opposite ends of the device, so long as the slider containing the silica or glass column can be moved from one chamber to the other.

This method and device can be coupled to numerous variants of existing sample capture and cell lysis techniques already described in this and earlier patent applications. This method could also be coupled to other sample capture and cell lysis techniques, so long as the composition of the sample immediately prior to beginning this process include high concentrations of salt and was in a practical pH range (for example, pH 3-12).

As described previously, the preferred embodiment of the device includes applying the sample to a porous support that contains a high concentration of chaotropic salt, which, among other functions, inactivates or kills agent in the sample. This effect renders the cartridge safe for subsequent handling and transport. For some applications, however, the user may want to culture any organisms present in the sample while still gaining the other advantages of processing the sample with chaotropic salt. Two alternate configurations of the sample cartridge address these conflicting goals are provided (see FIG. 38). In one design, a device with no chaotropic salt on the porous support is physically connected to a device with chaotropic salt. This connection allows the device with salt to be processed independently of the chaotropic salt-free device, while facilitating tracking of the sample by keeping the two parallel assays together. The chaotropic salt-free device may contain other chemicals that support viability of the organisms until culturing is possible.

In a second design, the inner chamber of a device is divided into two sub-chambers that have no fluidic communication. The porous support is also divided into two sections, with one section containing chaotropic salt while the other does not but instead may contain chemicals that enhance culture. This design is better suited for archival purposes, because both halves must be processed simultaneously. Although it is expected that it will be possible to culture from eluate taken from the chaotropic salt-free side of the inner cylinder, culturing from the porous support prior to elution will yield a higher concentration of organism.

### Example 17

### Isolation and Purification of RNA

As outlined in detail above, the similar characteristics and structure of DNA and RNA suggests that substrates that interact with DNA will also interact with RNA. The invention contemplates that the compositions and methods for the separation and/or identification of DNA from a sample can also be used for the identification and/or separation of RNA. However, given that RNA is typically less stable and more susceptible to degradation than DNA, the invention further contemplates that the separation and/or identification of RNA may require additional modifications to the present methods.

The ability to rapidly isolate and purify RNA from a sample of interest requires isolating the RNA under conditions that preserves the RNA. RNA is present in all organisms, so the methods described herein could be applied to RNA isolation from eukaryotes, prokaryotes, archaea, or viruses. In particular, we have explored isolation of RNA from viruses.

RNA isolation is complicated by the susceptibility of RNA to rapid degradation by nucleases in the environment. Viral RNA must be isolated from the virion particles in a way that inactivates these ribonucleases (RNases). Agents that inhibit or otherwise inactivate RNases are incorporated into many of the currently available laboratory procedures and commercial kits used to isolate RNA, however many of these methods are slow, labor intensive, and expensive.

We have previously reported the use of the SNAP method and the use of reagents such as IsoCode paper to help efficiently isolate DNA under conditions that inhibit the degradation of the DNA. Furthermore, we have previously reported the development of devices referred to as LiNK which incorporate SNAP methodology into a cartridge format for easier handling, portable, and field-related use. The present invention contemplates that SNAP and LiNK technologies can be adapted to further enhance ability to separate and analysis target RNA from a sample. Such RNA-focused modifications of SNAP and LiNK could be used alone, or could further enhance the efficacy of the Affinity Protocol described in the present application.

RNA-specific modifications of SNAP and LiNK technologies would be based on the following principles. Preservation of RNA should involve both the prevention of degradation of RNA by RNases, and the prevention of nonenzymatic hydrolysis of the phosphodiester bonds in RNA. This hydrolysis is mediated by high temperature or pH extremes and divalent cations. RNA purification, therefore, must take place in appropriately buffered solutions.

Identification of an RNA virus by reverse transcription PCR (RT-PCR) can be broken down into four steps: extraction and isolation of RNA, prevention of degradation of RNA by RNases and hydrolysis, conversion of RNA to cDNA via RT-PCR, and amplification of DNA via PCR. These steps are discussed in more detail below.

a) Extraction and Isolation of RNA

RNA isolation from viruses requires the dissociation of the external viral coatings without degradation of the RNA. Commonly used RNA-extraction methods include SDS, phenyl, or high-molarity chaotropic salt. IsoCode® paper, used in the SNAP protocol, also has the capability of releasing RNA from sample applied to the paper.

b) Prevention of RNA Degradation by RNases

Numerous RNase inhibitors exist. Many of these inhibitors could be used singly, or in combination for a rapid, simple RNA isolation protocol. Useful inhibitors must have a wide specificity (some RNase inhibitors act only against one class of RNases) and must not themselves inhibit downstream RT-PCR reactions (some RNase inhibitors are general enzyme inhibitors), or they need to be easily and completely removed from the extracted RNA.

The invention contemplates the following inhibitors for use in the separation and/or identification of RNA target: clays (bentonite, macaloid); aurintricarboxylic acid (ATA); chaotropic salts, including guanidinium thiocyanate (GT) and guanidinium hydrochloride (GH); diethylpyrocarbonate (DEPC); SDS; urea; and vanadyl-ribonucleoside complexes (VRCs).

The invention further contemplates that inhibition of hydrolysis by pH and temperature extremes can be mediated by eluting RNA in pH-buffered solutions such as Tris-EDTA.

The following RNase inhibitors have characteristics that make them preferred agents for use in the methods of the present invention: macaloid, bentonite, ATA, SDS, urea, DEPC, and the chaotropic salts. These agents are stable at room temperature, and either do not inhibit downstream RT and PCR reactions or are easily removed or diluted without organic extraction. The following paragraphs provide brief descriptions of each of these inhibitors.

Overview of RNase Inhibitors

Two of the RNase inhibitors, macaloid and bentonite, are types of clay. Their inhibitory properties are thought to be caused by their overall negative charge, which allows them to bind RNases and other basic proteins. Macaloid is a purified hectorite (a clay consisting of sodium magnesium lithofluorosilicate). Bentonite is a montmorillonite clay (Al₂O₃.5SiO₂.7H₂O). A fraction prepared from each of the clays is stable at room temperature and appears to be compatible with incorporation into a cartridge format. They have different pH optima for RNase inhibition and so could be used separately or together.

Aurintricarboxylic acid (ATA) is a general inhibitor of nucleases (DNases and RNases, included) in in vitro assays, and has been used in bacterial RNA isolation. ATA is the primary constituent of a commercial RNase inhibitor, RNase block (Innogenex, Inc.). It is a highly water soluble, dark red solution that can be removed from purified nucleic acids by gel filtration (through Sephadex G-100). RNA isolated with ATA can be used for RT-PCR. ATA does not appear to inhibit DNA isolation, however trace amounts may inhibit the action of reverse transcriptases. If such inhibition of reverse transcriptases is observed, an extraction step to eliminate the ATA prior to reverse transcription may be readily employed.

Chaotropic salts such as the guanidinium compounds (GT and GH) are strong protein denaturants that inhibit the action of RNases and are the basis of many RNA extraction procedures. These compounds are the basis of the IsoCode® paper that is used in the SNAP protocol.

Vanadyl-ribonucleoside complexes (VRCs) are competitive inhibitors of RNases. They are superior to DEPC, polyvinyl sulfate, heparin, bentonite, macaloid, SDS, and proteinase K. Unfortunately, they have significant drawbacks in that trace amounts inhibit RT and PCR polymerase activity, requiring removal by organic extraction. Additionally, VRCs do not inhibit all RNases, and specifically do not inhibit the activity of RNase H. A further, although not insurmountable, limitation is that VRC require storage at <−20° C. We note however, that the physical attachment of VRCs to a particular surface (for example, a cartridge over which a sample is passed or a bead which can be added and removed from a sample) would enable binding of RNases by mixing the sample in the presence of the modified surface and subsequent physical separation of VRCs from the sample prior to subsequent molecular analysis.

SDS is a detergent that denatures proteins, including RNases.

For any of the foregoing, as with all currently employed RNA-isolation procedures, relevant solutions will be pretreated with DEPC. DEPC is not useful as a standalone RNase inhibitor for environmental samples as it reacts with amines and becomes inactivated.

c) Reverse Transcription and PCR

The extracted RNA must be compatible with downstream analysis, i.e. free of reverse-transcriptase and PCR inhibitors. As reviewed in Wilson, 1997, materials to remove inhibitors include 5% DMSO, BSA, and the T4 Gene 32, among others. In addition, RT-PCR reaction conditions are available for the detection of many viruses of interest (De Paula, 2002; Drosten, 2002; Leroy, 2000; Pfeffer, 2002; Warrilow, 2002).

One application of the above outlined methodologies for separating and further analyzing target RNA is in the construction of devices which incorporate reagents which help prevent the degradation of target RNA and/or prevent the action of compounds which inhibit the later molecular analysis of an RNA target. Such devices and methodologies can be used alone or in combination with methods and devices based on the Affinity Protocol described herein.

The following provides a detailed description of an exemplary layered device. However, the invention contemplates the construction of devices that utilize the same or similar reagents but are not organized in a layered configuration. Construction of a device or development of a cartridge approach into which a sample is placed could be done in a layered approach as follows:

a) Lysis of the Organism of Interest

The part of the device which first contacts the sample could contain reagents to lyse viruses, bacteria, eukaryotic, or archaeal organisms. This lysis will split the organism open and allow DNA or RNA to be extracted. Reagents to do this could consist of chaotropic salts, SDS, or urea. Additionally, heat or cold could be used to lyse samples. Temperature changes could be provided by a battery-powered resistor-based heating circuit built into the support structure for a cartridge or by means of a chemical reaction.

Possible implementations of the lysis mechanism could include addition of solutions containing the aforementioned reagents; addition of the sample to a dry filter or matrix containing those reagents, which upon the addition of water (for a dry sample) or the sample itself (for a liquid sample), the reagents would re-dissolve to the correct concentration.

b) Inhibition of RNases

Intermixed with the reagents to lyse the sample, reagents to inhibit the action of RNases, to physically trap the RNases, or to bind the RNases should be present. These reagents include GT, GH, urea, SDS, bentonite, macaloid, ATA, VRCs, and cellulose-based papers like IsoCode®. GT, GH, urea, and SDS can be present in solution and can be removed by the addition of a desalting step or dilution to a concentration that doesn't inhibit the action of downstream detection steps. The clays bentonite and macaloid can be layered on top of IsoCode® or other cellulose-based papers. Incorporation of ATA or VRCs can be done by chemically linking the ATA or VRCs to a solid support, so that they are not present in the eluate that contains RNA, or by addition of a filtration step.

c) Filtration to Remove ATA

In the event that the device incorporates ATA as an RNase inhibitor, it is necessary to remove the ATA from the eluate. This can be done by filtration through a size exclusion column (e.g., a Sephadex G-100 column). Such a column could be included as a layer in a cartridge-based device.

d) Binding of Nucleic Acid and Removal of RNases

A layer of size-fractionated silica, chemically-treated beads, or a chemically treated membrane or surface can be used to bind nucleic acids (DNA or RNA) to allow subsequent purification by rinsing the lysed sample to remove metals, salts, or other materials that have not been specifically bound in the previous layers. Nucleic acids can then be eluted from the silica, beads, or surface with appropriate conditions and analyzed using standard methods in molecular biology.

### Example 18

### Simultaneous Detection of Multiple Targets

For many applications of the present invention, the ability to simultaneously assess the presence of multiple target is advantageous. For example, the ability to separate two different bacterial cell types would enable medical diagnostics that assess the presence of multiple, potentially infectious agents in a single test. Similarly, the ability to separate both DNA and RNA from the same sample would allow simultaneous assessment of bacterial and viral organisms, or of DNA and RNA-based viruses.

We evaluated the ability to isolate DNA and RNA using a commercially available glass fiber filter, and a standard protocol for the use of this filter. Our results indicated that DNA and RNA can be simultaneously isolated from the same sample using standard protocols and indicated that simultaneous isolation of multiple targets using the Affinity Protocol is also possible. The use of the Affinity Protocol would greatly simplify separation of multiple agents in comparison to currently available techniques which are more time, labor, and reagent intensive.

Briefly, samples containing bacteria (bacillus thuringiensis-Btk), MS2 bacteriophage (a bacteriophage that infects E. coli and serves as a model for single-stranded, RNA viruses), or both Btk and MS2 were analyzed. Samples were diluted in L6 buffer (buffer containing: guanidine isothiocyanate; 0.1M Tris-HCl (pH 6.5); 0.2M EDTA (pH 8.0); Triton-X 100) and passed over a commercially available, glass fiber filter in a volume of 1 mL. 60 mL of air was passed through the filter using a 60 mL syringe. 2 mL of L2 buffer (buffer containing: guanidine isothiocyanate; 0.1M Tris-HCl (pH 6.5); 0.2M EDTA (pH 8.0); Triton-X 100) was applied to the filter. Application of L2 buffer was followed by 60 mL of forced air, 3 mL of 70% EtOH, and then another 60 mL of forced air (repeated 2×). The filter was then dried, and target was eluted with TE (Tris, 1.0 mM EDTA−final pH=7.0).

RT-PCR and PCR were performed on aliquots of the eluate to detect viral RNA and bacterial DNA, respectively. RT-PCR was performed in a reaction volume of 25 μl. A One-Step RT-PCR Reaction (TaMan One-Step, Applied Biosystems) was prepared using an MS2 specific primer and probe set and run in an ABI7700 real-time PCR machine (Applied Biosystems). Each 25 μl reaction contained 2.5 μl of sample eluate. The following RT-PCR conditions were used: 30 minutes at 48° C., 10 minutes at 95° C., 50 cycles of 15 seconds each at 95° C., and 1 minute at 60° C. PCR was similarly performed, however, Btk specific primers were used.

The presence of MS2 was detected by RT-PCR in samples containing either MS2 alone or a combination of MS2 and Btk. Detection of MS2 by RT-PCR in samples containing only MS2 occurred with a cycle threshold of 20.65 (standard deviation=0.33). Detection of MS2 by RT-PCR in samples containing both MS2 and Btk occurred with a cycle threshold of 21.75 (standard deviation=2.04).

The presence of Btk was detected by PCR in samples containing either Btk alone or a combination of Btk and MS2. Detection of Btk by PCR in samples containing only Btk occurred with a cycle threshold of 23.65 (standard deviation=0.23). Detection of Btk by PCR in samples containing both Btk and MS2 occurred with a cycle threshold of 23.81 (standard deviation=0.39).

### Example 19

### Separation and Identification of RNA Targets

Although commercially available glass-fiber filters, and the accompanying methodologies, can be used to separate DNA and RNA targets. These methods are time and reagent intensive, and thus present limitations to (i) their use in the field; (ii) their use for time-sensitive applications; (iii) their use for cost-sensitive applications. As outlined in detail in the present application, the Affinity Protocol overcomes many of the limitations of other analytical methods known in the art and allows separation and, optionally, further analysis of a variety of targets with minimal reagents and time.

We have demonstrated that the Affinity Protocol can be effectively used to separate a variety of targets including bacterial cells and bacterial spores, and additionally that DNA from bacterial cells and spores separated by the Affinity Protocol can be further analyzed by methods such as PCR. We now show that the Affinity Protocol can be effectively used to separate viral targets, and additionally that RNA from viral targets separated by the Affinity Protocol can be further analyzed by methods such as RT-PCR.

MS2 was separated from a sample of water using either a commercially available, glass fiber filter and the manufacturers instructions (as outlined in Example 18), or using the Affinity Magnet Protocol (amine derivatized magnetic beads for target capture and elution in buffer containing 100 ug/ml of calf thymus DNA in 0.01N NaOH). Following separation of MS2 using either method, eluate was processed by RT-PCR to identify MS2 RNA. Briefly, we successfully separated and further analyzed by RT-PCR MS2 using either methodology. Detection of MS2 by RT-PCR following separation of MS2 using the glass fiber filter occurred with a cycle threshold of 29.83 (standard deviation=0.19). Detection of MS2 by RT-PCR following separation of MS2 using the Affinity Protocol occurred with a cycle threshold of 33.02 (standard deviation=0.72). Although sensitivity of detection appears slightly higher following separation using the glass fiber filter, significant improvements with respect to time, cost, and ease of operation are achieved using the Affinity Protocol.

Further experiments indicated that the differences in sensitivity in the detection of RNA following separation using the glass fiber filter method versus the Affinity Protocol were due to an inhibitory effect on RT-PCR analysis, and not due to inefficient capture or elution of target using the Affinity Protocol. Briefly, prior to RT-PCR analysis, MS2 containing eluate was diluted in either water or in AP-elution buffer and incubated for 0, 30, or 60 minutes prior to RT-PCR analysis of MS2. Detection of MS2 by RT-PCR following incubation of the sample in water for 0, 30, or 60 minutes occurred with a cycle threshold of 20.57, 20.65, and 21.02, respectively (standard deviation=NA). Detection of MS2 by RT-PCR following incubation of the sample in elution buffer for 0, 30, or 60 minutes occurred with a cycle threshold of 24.15, 24.05, and 24.14, respectively (standard deviation=0.03, 0.93, and 0.04, respectively).

Additional References

U.S. Pat. No. 5,665,582
U.S. Pat. No. 5,935,858
U.S. Pat. No. 5,705,628
U.S. Pat. No. 6,057,096
U.S. Pat. No. 5,945,525
U.S. Pat. No. 5,695,989
http://www.spie.org/Conferences/Progams/03/pe/eis/index.cfm?fuseaction=5271A
http://www.grc.uri.edu/programs/2003/antimicr.htm
http://216.239.39.104/search?q=cache:F4PegGNWHKIJ:journals.tubitak.gov.tr/biology/i ssues/biy-02-26-4/biy-26-4-3-0205-3.pdf+antimicrobial+peptides+&hI=en&ie=UTF-8
http://www.nature.com/cgi-taf/DynaPage.taf?file=/nature/journal/v415/n6870/full/415389a_r.html
Hamaguchi et al. (2001) Anal. Biochem. 294: 126-131.
Cox et al. (2001) Bioorgan. Med. Chem. 9: 2525-2531.
(2003) JACS 125(14): 4111-4118.
(2003) Analyst 128(6): 681-685.
(2002) Lett. Pept. Sci. 9(4): 211-219.
Sotlar et al. (2003) Am. J. Path. 162(3): 737-746.
Chandler and Jarrell. (2003) Anal. Biochem. 312(2): 182-190.
Batt (1996) Use of IsoCode® Stix for the preparation of blood samples destined for allelic PCR-based assays. Applications Note #644, Schleicher & Schuell.
Berger and Birkenmeier (1979) Biochemistry 18: 5143-5149.
Boom et al. (1990). J. Clin. Microbiol. 28: 495-503.
Chomczynski and Sacchi (1987) Anal. Biochem.162: 156-159.
Cox (1968) Methods Enzymol. 12B: 120-129.
De Paula et al. (2002) Trans. R. Soc. Trop. Med. Hvg. 96: 266-269.
Drosten et al. (2002) J. Clin. Microbiol. 40: 2323-2330.
Givens and Manly (1976) Nucleic Acids Res. 3: 405-418.
Griffin et al. (1978) Anal. Biochem. 87: 506-520.
Gonzalez et al. (1980) Biochemistry 19: 4299-4303.
Fraenkel-Conrat et al. (1961) Virology 14: 54.
Hallick et al. (1977) Nucleic Acids Res. 9: 3055-3064.
Kreader (1996) Appl. Environ. Microbiol. 62: 1102-1106.
Lau et al. (1993) Nucleic Acids Res. 21: 2777.
Leroy et al. (2000) J. Med. Virol. 60: 463-467.
Lönneborg and Jensen (2000) BioTechniques. 29: 714-718.
Masoud et al. (1992) PCR Methods Appl. 2: 89-90.
Marcus L, Halvorson H O. 1968. in Methods in Enzymology. 12 (part A), 498-.
Nakane et al. (1988) Eur. J. Biochem. 177: 91-96.
Pasloske (2001) Methods Mol. Biol. 160: 105-111.
Pfeffer et al. (2002) J. Vet. Med. B Infect. Dis. Vet. Public Health. 49: 49-54.
Poulson, (1977) Isolation, purification, and fractionation of RNA. In The Ribonucleic Acids, 2^{nd} Ed. Stewart P R, Letham D S. eds. Springer-Verlag, New York, pp 333-361.
Reddy et al. (1990) BioTechniques 8: 250-251.
Sidhu et al. (1996) BioTechniques 21: 44-47.
Singer and Fraenkel-Conrat (1961) Virology 14: 59.
Su et al. (1997) BioTechniques 22: 1107-1113.
Tyulkina and Mankin (1984) Anal. Biochem. 138: 285-290.
Warrilow et al. (2002) J. Med. Virol. 66: 524-528.
Wilson (1997) Appl. Environ. Microbiol. 63: 3741-3751.
Al-Soud and Radstrom (2001) Journal of Clinical Micorbiology 39: 485-493.
VanElden et al. (2001) Journal of Clinical Micorbiology 39: 196-200.

All publications, patents and patent applications are herein incorporated by reference in their entirety to the same extent as if each individual publication, patent or patent application was specifically and individually indicated to be incorporated by reference in its entirety.

### Equivalents

Those skilled in the art will recognize, or be able to ascertain using no more than routine experimentation, many equivalents to the specific embodiments of the invention described herein.

1. A method of separating a target from a heterogeneous sample, comprising
(a) contacting said sample with a substrate for a time sufficient for said substrate to bind said target to form a substrate-target complex, which substrate is modified with one or more surface modifying agents to form a surface modified substrate and which substrate binds to said target with higher affinity than to non-target materials;
(b) removing said substrate-target complex from said sample, thereby separating said target from said heterogeneous sample.

2. The method of claim 1, wherein said time sufficient to form said substrate-target complex is less than 15 minutes.

3. The method of claim 1, wherein said time sufficient to form said substrate-target complex is less than 5 minutes.

4. (canceled)

5. The method of claim 1, wherein said one or more surface modifying agents are selected from the agents represented in any of FIG. 2, FIG. 3, or FIG. 10, and wherein the surface modified substrate binds to said target with higher affinity than to non-target materials.

6. The method of claim 5, wherein the substrate is a magnetic or paramagnetic substrate.

7. The method of claim 6, wherein the one or more surface modifying agents is appended to the substrate via a cleavable linker.

8-9. (canceled)

10. The method of claim 1, wherein said heterogeneous sample is a biological sample.

11-12. (canceled)

13. The method of claim 1, further comprising
(c) contacting said substrate-target complex with elution buffer for a time sufficient to elute said target from said substrate, thereby separating said target from said substrate.

14. (canceled)

15. The method of claim 13, wherein said time sufficient to elute said target from said substrate is less than 5 minutes.

16. The method of claim 15, wherein said time sufficient to elute said target from said substrate is less than 1 minute.

17. (canceled)

18. A method of separating target from a heterogeneous sample, comprising
(a) contacting said sample with a substrate for a time sufficient for said substrate to bind said target to form a substrate-target complex, which substrate binds to said target with higher affinity than to non-target materials;
(b) removing said substrate-target complex from said sample, thereby separating said target from said heterogeneous sample;
(c) contacting said substrate-target complex with elution buffer for a time sufficient to elute said target from said substrate, thereby separating said target from said substrate;
wherein said target comprises DNA, RNA, protein, eukaryotic cells, archaea, bacterial cells or spores, viruses, small organic molecules, or chemical compounds, and wherein said method of separating comprises separating DNA, RNA, protein, eukaryotic cells, archaea, bacterial cells or spores, viruses, small organic molecules, or chemical compounds from a heterogeneous sample.

19-20. (canceled)

21. The method of claim 18, wherein said time sufficient to form said substrate-target complex is less than 15 minutes.

22. The method of claim 21, wherein said time sufficient to form said substrate-target complex is less than 5 minutes.

23. The method of claim 18, wherein said substrate is modified with one or more surface modifying agents to form a surface modified substrate.

24. The method of claim 23, wherein said one or more surface modifying agents are selected from the agents represented in any of FIG. 2, FIG. 3, or FIG. 10, and wherein the surface modified substrate binds to one or more targets with higher affinity than to non-target materials.

25. The method of claim 18, wherein the substrate is a magnetic or paramagnetic substrate.

26. The method of claim 25, wherein the one or more surface modifying agents is appended to the substrate via a cleavable linker.

27. (canceled)

28. The method of claim 18, wherein said time sufficient to elute said target from said substrate is less than 5 minutes.

29. The method of claim 28, wherein said time sufficient to elute said target from said substrate is less than 1 minute.

30. A substrate modified with one or more surface modifying agents to form a surface modified substrate, wherein the one or more surface modifying agents are selected from the agents represented in any of FIG. 2, FIG. 3, or FIG. 10, and wherein the surface modified substrate binds to one or more targets with higher affinity than to non-target materials.

31. The surface modified substrate of claim 30, wherein the substrate is a magnetic or paramagnetic substrate.

32. The surface modified substrate of claim 30, wherein the one or more surface modifying agents is appended to the substrate via a cleavable linker.

33. The surface modified substrate of claim 30, wherein the surface modified substrate binds to DNA, RNA, a protein, a small organic molecule, or a chemical compound.

34. The surface modified substrate of claim 30, wherein the surface modified substrate binds to a eukaryotic cell, archaea, bacterial cell or spore, or viral particle from one or more species.

35-37. (canceled)

38. The substrate of claim 30, wherein said substrate is a bead, and wherein said bead has a particle size of 0.1-120 μm.

39. The substrate of claim 30, wherein said substrate has a diameter of 0.5-10 mm.

40. The substrate of claim 30, wherein said substrate is a tube or culture vessel.

41. A filter, comprising one or more layers, wherein at least one of said one or more layers comprises one or more substrates, and wherein said one or more substrates are modified with one or more surface modifying agents to form the surface modified substrate of claim 30.

42. The filter of claim 41, wherein said filter comprises one layer comprising one or more substrates, and wherein said substrates are modified with multiple surface modifying agents.

43. (canceled)

44. The surface modified substrate of claim 30, wherein said substrate is modified with two or more surface modifying agents.

45. A cartridge, comprising the surface modified substrate of claim 30.

46. A method of releasing a target, wherein said target is bound to a substrate to form a target-substrate complex, comprising contacting said target-substrate complex with an elution buffer for a period of time, which period of time is an elution time, thereby disrupting said target-substrate complex and releasing said target from said substrate.

47. The method of claim 46, wherein said elution buffer contains calf thymus DNA.

48. The method of claim 46, wherein said elution buffer has a pH of approximately pH 11-13.

49. The method of claim 48, wherein said elution buffer has a pH of approximately pH 11.5-12.3

50. (canceled)

51. The method of claim 46, wherein said elution time is 1-5 minutes.

52. The method of claim 51, wherein said elution time is less than 1 minute.

53. A method of capturing a target, comprising contacting a sample containing said target with an amount of substrate and for a period of time, which period of time is a capture time, sufficient to capture target and form a target-substrate complex, wherein said capture time is 1-10 minutes.

54. The method of claim 53, wherein said capture time is 1-5 minutes.

55. The method of claim 54, wherein said capture time is less than 1 minute.

56. The method of claim 53, wherein said amount of substrate is approximately 1-5 mg/mL of sample.

57. The method of claim 56, wherein said amount of substrate is approximately 1 mg/mL of sample.

58. The method of claim 57, wherein said amount of substrate is less than 1 mg/mL of sample.

59-66. (canceled)
